SEQUENCE LISTING (74 sequences)

SEQ ID NO: 1 - DNA, 13219 bp, artificial sequence
  Gene expression cassette of pDAB1585
agcttcggatttggagccaagtctcataaacgccattgtggaagaaagtcttgagttggtggtaatgtaacagagtagtaagaacagagaagagagagagtgtgagatacatgaattgtcgggcaacaaaaatcctgaacatcttattttagcaaagagaaagattccgagtctgtagcagaagagtgaggagaaatttaagctcttggacttgtgaattgttccgcctcttgaatacttcttcaatcctcatatattcttcttctatgttacctgaaaaccggcatttaatctcgcgggtttattccggttcaacattttttttgttttgagttattatctgggcttaataacgcaggcctgaaataaattcaaggcccaactgtttttttttttaagaagttgctgttaaaaaaaaaaaagggaattaacaacaacaacaaaaaaagataaagaaaataataacaattactttaattgtagactaaaaaaacatagattttatcatgaaaaaaagagaaaagaaataaaaacttggatcaaaaaaaaaaacatacagatcttctaattattaacttttcttaaaaattaggtcctttttcccaacaattaggtttagagttttggaattaaaccaaaaagattgttctaaaaaatactcaaatttggtagataagtttccttattttaattagtcaatggtagatacttttttttcttttctttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcagccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtaagagctcgcatgcggtcaccaaaccttggactcccatgttggcaaaggcaaccaaacaaacaatgaatgatccgctcctgcatatggggcggtttgagtatttcaactgccatttgggctgaattgaagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatcgcgatagtactacgaatatttcgtatcgctgatcttctcaatcacaatgatgcgtagtgacccgacaaataatttaagcgtccttaataccaatcctaaaataattgaggcaaataaaatttttttgtaattcttatgatagcagatcgattctccagcaagcctgcaacaaaatattgtgtatttctaaatagattttgatattaaaatcccgagaaagcaaaattgcatttaacaaaacagtaatttagtacattaataaaaattatgctcggccggccgcggccaaacggatcctaaccggtgtgatcatgggccgcgattaaaaatctcaattatatttggtctaatttagtttggtattgagtaaaacaaattcgaaccaaaccaaaatataaatatatagtttttatatatatgcctttaagactttttatagaattttctttaaaaaatatctagaaatatttgcgactcttctggcatgtaatatttcgttaaatatgaagtgctccatttttattaactttaaataattggttgtacgatcactttcttatcaagtgttactaaaatgcgtcaatctctttgttcttccatattcatatgtcaaaacctatcaaaattcttatatatctttttcgaatttgaagtgaaatttcgataatttaaaattaaatagaacatatcattatttaggtatcatattgatttttatacttaattactaaatttggttaactttgaaagtgtacatcaacgaaaaattagtcaaacgactaaaataaataaatatcatgtgttattaagaaaattctcctataagaatattttaatagatcatatgtttgtaaaaaaaattaatttttactaacacatatatttacttatcaaaaatttgacaaagtaagattaaaataatattcatctaacaaaaaaaaaaccagaaaatgctgaaaacccggcaaaaccgaaccaatccaaaccgatatagttggtttggtttgattttgatataaaccgaaccaactcggtccatttgcacccctaatcataatagctttaatatttcaagatattattaagttaacgttgtcaatatcctggaaattttgcaaaatgaatcaagcctatatggctgtaatatgaatttaaaagcagctcgatgtggtggtaatatgtaatttacttgattctaaaaaaatatcccaagtattaataatttctgctaggaagaaggttagctacgatttacagcaaagccagaatacaatgaaccataaagtgattgaagctcgaaatatacgaaggaacaaatatttttaaaaaaatacgcaatgacttggaacaaaagaaagtgatatattttttgttcttaaacaagcatcccctctaaagaatggcagttttcctttgcatgtaactattatgctcccttcgttacaaaaattttggactactattgggaacttcttctgaaaatagtgggggtaccgagttcttgtacaccagtacaagaactcggaggtgttctccgattcgctgcgagataccgagttcttgtacaccagtacaagaactcggcacgggatacttgtagaggtacccacgccgagttcttgtacaccagtacaagaactcgggtggcgctaacagaaggatttccaccaccgagttcttgtacaccagtacaagaactcggctcccccaccgcttaattaaggcgcgccatgcccgggcaagcggccgcattcccgggaagctaggccaccgtggcccgcctgcaggggaagcttgagggtgtggaagatatgaatttttttgagaaactagataagattaatgaatatcggtgttttggttttttcttgtggccgtctttgtttatattgagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgtctagggtcgaggggggatccgtcgagggggtccaccaaaaacgtaagcgcttacgtacatggtcgagggggtccaccaaaaacgtaagcgcttacgtacatggtcgagggggtccaccaaaaacgtaagcgcttacgtacatggtcgagggggtccaccaaaaacgtaagcgcttacgtacatggtcgactagagcgtgacgctcgcggtgacgccatttcgccttttcagaaatggataaatagccttgcttcctattatatcttcccaaattaccaatacattacactagcatctgaatttcataaccaatctcgatacaccaaatcccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggcccttgaaggggccgcattcccgggaagctaggccaccgtggcccgcctgcaggggaagcttgtttaaacccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtagatctccatggcattatccgagttcaccagaactcggataatggcaccttcatcacaggcaccttcagcattatccgagttcaccagaactcggataatgggttctgctccaggacctggcgctgcattatccgagttcaccagaactcggataatgggtccatcgcctggacctccatcagcattatccgagttcaccagaactcggataatggacatggtaaggggcagccaccaccaccaccaccacatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgataatgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggtttggcctaggccacggtggccagatccactagttctagagcggcccctcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagaccccgatcgcattcgcctaatgagagctcgtttaaacagatcggcggcaatagcttcttagcgccatcccgggttgatcctatctgtgttgaaatagttgcggtgggcaaggctctctttcagaaagacaggcggccaaaggaacccaaggtgaggtgggctatggctctcagttccttgtggaagcgcttggtctaaggtgcagaggtgttagcggggatgaagcaaaagtgtccgattgtaacaagatatgttgatcctacgtaaggatattaaagtatgtattcatcactaatataatcagtgtattccaatatgtactacgatttccaatgtctttattgtcgccgtatgcaatcggcgtcacaaaataatccccggtgactttcttttaatccaggatgaaataatatgttattataatttttgcgatttggtccgttataggaattgaagtgtgcttgcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataggccggcctaggccacggtggccagatccactagttctagagcggccgcttaattaaatttgggtaccgagttcttgtacaccagtacaagaactcggaggtgttctccgattcgctgcgagataccgagttcttgtacaccagtacaagaactcggcacgggatacttgtagaggtacccacgccgagttcttgtacaccagtacaagaactcgggtggcgctaacagaaggatttccaccaccgagttcttgtacaccagtacaagaactcggctcccaaatgtttgatgcttaccatgggtcagttttacttcccttaattttctatgtactttcataattacttatgttattttcttcatgagttttaatgcaaattactatatggactctagtgaaaacgttcagaatcctataaacatgactactgagacgaacttgagagtagttttgatcatacacacgtttcatgtggtacttgagagttactaatttttgtcatcttcgtataagtagtaaaagatactacaagaatagtttagtagaaaatactagcggtaggtgaagatttgtcgctatgtactattattgtctagtaacttgagtaacaatttcgtggtctaaatatcaaataaaaatggatgagtggttcaccaaatctaggcatcaaaactattaatgtcattgtctagatcttaggtgacaccacatttcgaatatttattggtaattgagatgttaaagtaccaatatttgacttaataaactaaaagattttggctttatcaaatgtagacattgatgacatatcgttgtcattatcttgagtatatacaagtcgatcaattaggtgaaagtttagtgtctcgtggttggtaaacgattaatacagtagtatattttatccaaagacaaaatccaaatcatttcaccagtatgaatagtattattttatcttaaaagctaaaatcttaaaaaccaaggtagcacccacgttgagctagacgatcaaatcgatttctgctttgtccaatttaccaagctatttaaagccaaataattgaaatataggtaggtcgttatattaggctaagatttatctcaaatgcttaactaaaggaataacaagggattctagttgtgtggttttataagattggtccaatttcacttaagtttgtttattgtagaattttatatgtgaataatttgaattccaattgaaaagatattatagtaaaagaaaaaatagtgcgaacaaaaaactttaatcccataaaaagaaaaagaaaaatgaaaagttcttctaacatccatattttgcatcatatcataaagataagaaagatacatatcatagacgtacagataaacaaacatatcatcatttgtgaaatacatagtacaataatttgcttttaaatagagtttaagtcacacacactgacacacacgataaaacgataatgtctgcaaaaacactttaatcccattgcctagaggacagcttctccactttgtctttaaggttggttttgccgtgttgtttttatctttatataatgatctattttttggattatgaaatgaattcacacattttaattatttaagaagatccatatacaggtttataacagtactaagtgatgattattttttgtttttgcatagtttagtttattgggtaaacattcattacgtgtctctttatacgaatcacccatccaaaatttcaagtagtcttttagttcatttattatttcataactatttgacttattgatttgacaagaaacaacaaaagtgttgacttattgatagattgtgggatcataaaagtaattaagcgtcaaccacgacccacaacaacaaagcacatgttatacattaatatctcgtttacttaattacagttttcagaatgccgtttcatgtcttcgtcactggcgatgttattatcatgttggacaatattcgactgttgtcgtttttacattttcgtattgactaaaactaaaaaaacaaaactctgtttcaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgaggtaccctgagctctgtccaacagtctcagggttaatgtctatgtatcttaaataatgttgtcggtattttgtaatctcatatagattttcactgtgcgacgcaaaaatattaaataaatattattattatctacgttttgattgagatatcatcaatattataataaaaatatccattaaacacgatttgatacaaatgacagtcaataatctgatttgaatatttattaattgtaacgaattacataaagatcgaatagaaaatactgcactgcaaatgaaaattaacacatactaataaatgcgtcaaatatctttgccaagatcaagcggagtgagggcctcatatccggtctcagttacaagcacggtatccccgaagcgcgctccaccaatgccctcgacatagatgccgggctcgacgctgaggacattgcctaccttgagcatggtctcagcgccggctttaagctcaatcccatcccaatctgaatatcctatcccgcgcccagtccggtgtaagaacgggtctgtccatccacctctgttgactacagccactgcagccgcatggacctcacgtgcca

SEQ ID NO: 2 - DNA, 4137 bp, artificial sequence
  Gene expression cassette of pDAB118259
ttgagtaaaacaaattcggcgccatgcccgggcaagcggccgcacaagtttgtacaaaaaagcaggctgagtattcactctagacctaggtagccgagttcttgtacaccactacaagaactcggaggtgttctccgattcgctgcgagataccgagttcttgtacaccactacaagaactcggcacgggatacttgtagaggtacccacgccgagttcttgtacaccactacaagaactcggtaagcttctcgagctttgatgcctatgtgacacgtaaacagtactctcaactgtccaatcgtaagcgttcctagccttccagggcccagcgtaagcaataccagccacaacaccctcaacctcagcaaccaaccaagggtatctatcttgcaacctctctagatcatcaatccactcttgtggtgtttgtggctctgtcctaaagttcactgtagacgtctcaatgtaatggttaacgatatcacaaaccgcggccatatcagctgctgtagctggcctaatctcaactggtctcctctccggagaagccatggttggatccttacctgttaatcagaaaaactcagattaatcgacaaattcgatcgcacaaactagaaactaacaccagatctagatagaaatcacaaatcgaagagtaattattcgacaaaactcaaattatttgaacaaatcggatgatatttatgaaaccctaatcgagaattaagatgatatctaacgatcaaacccagaaaatcgtcttcgatctaagattaacagaatctaaaccaaagaacatatacgaaattgggatcgaacgaaaacaaaatcgaagattttgagagaataaggaacacagaaatttaccttgatcacggtagagagaattgagagaaagtttttaagattttgagaaattgaaatctgaattgtgaagaagaagctttgggtattgttttatagaagaagaagaagaaaagacgaggacgactaggtcacgagaaagctaaggcggtgaagcaatagctaataataaaatgacacgtgtattgagcgttgtttacacgcaaagttgtttttggctaattgccttatttttaggttgaggaaaagtatttgtgctttgagttgataaacacgactcgtgtgtgccggctgcaaccactttgacgccgtttattactgactcgtcgacaaccacaatttctaacggtcgtcataagatccagccgttgagatttaacgatcgttacgatttatatttttttagcattatcgttttattttttaaatatacggtggagctgaaaattggcaataattgaaccgtgggtcccactgcattgaagcgtatttcgtattttctagaattcttcgtgctttatttcttttcctttttgtttttttttgccatttatctaatgcaagtgggcttataaaatcagtgaatttcttggaaaagtaacttctttatcgtataacatattgtgaaattatccatttcttttaattttttagtgttattggatatttttgtatgattattgatttgcataggataatgacttttgtatcaagttggtgaacaagtctcgttaaaaaaggcaagtggtttggtgactcgatttattcttgttatttaattcatatatcaatggatcttatttggggcctggtccatatttaacactcgtgttcagtccaatgaccaataatattttttcattaataacaatgtaacaagaatgatacacaaaacattctttgaataagttcgctatgaagaagggaacttatccggtcctagatcatcagttcatacaaacctccatagagttcaacatcttaaacaagaatatcctgatccgttgacctgcaggtcgacaccggtccgagttcttgtacaccactacaagaactcggaggtgttctccgattcgctgcgagataccgagttcttgtacaccactacaagaactcggcacgggatacttgtagaggtacccacgccgagttcttgtacaccactacaagaactcggtactagtgctagccttgtcgacatttaaatgatgagtcggacccagctttcttgtacaaagtggttgcggccgcttaataagcttcttgcctcaattccggaggtgtttctagtgttcaacatgacaaacaaaacccatctctttcagtatatgtctctcagttgtgcttaattcaaatttcaactcagagaacttcttggcatacttatccagattatctaatgatctcatctaatggtaattcaactttcagtatatgtctcgcagcaaactatctttacatcaaatttttaacaactcaatgcacaaaatacttttcctcaacctaaaaataaggcaattagccaaaaacaactttgcgtgtgaacaacgcgttacacgtccctacacatacgtgtcaatttataattggctattgcttccacgccttagctttctcgtgaccgaccgagtcgtcctcgtcttttttgcttctataaatcaaatacccaaagagctcttcttcttcacaattcagattccaattttctcaaactctaaaatcaatctctcaaatctctcaaccgtgatcaaggtagatttctgagttcttattgtatttcttcgatttgtttcgttcgatcgcaatttaggctctgttctttgattttgatctcgttaatctctgatcggaggcaaattacatagtttcatcgttagatctcttcttatttctcgattagggttcgtatttttcgcagatctgtttattttcttgttgtttccttgtatttgatccgatttgttgaaagaatttgtgtgttctcgattatttacgctttgatctgtgatttttatctagatttggtgttagtttcttgtttgtgcgatcgaatttgtcgattaatctcggtttttctgattaacagggatccaaccatggatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagagctcggtaacctttaaactgagggcactgaagtcgcttgatgtgctgaattgtttgtgatgttggtggcgtattttgtttaaataagtaagcatggctgtgattttatcatatgatcgatctttggggttttatttaacacattgtaaaatgtgtatctattaataactcaatgtataagatgtgttcattcttcggttgccatagatctgcttatttgacctgtgatgttttgactccaaaaaccaaaatcacaactcaataaactcatggaatatgtccacctgtttcttgaagagttcatctaccattccagttggcatttatcagtgttgcagcggcgctgtgctttgtaacataacaattgttacggcatatatccaa

SEQ ID NO: 3 - DNA, 7339 bp, artificial sequence
  Gene expression cassette of pDAB118257
gagtaaaacaaattcggcgccatgcccgggcaagcggccgcacaagtttgtacaaaaaagcaggctgagtattcactctagacctaggtagccgagttcttgtacaccactacaagaactcggaggtgttctccgattcgctgcgagataccgagttcttgtacaccactacaagaactcggcacgggatacttgtagaggtacccacgccgagttcttgtacaccactacaagaactcggtaagcttctcgagcgatcgacgcccgggctgaaacagagttttgtttttttagttttagtcaatacgaaaatgtaaaaacgacaacagtcgaatattgtccaacatgataataacatcgccagtgacgaagacatgaaacggcattctgaaaactgtaattaagtaaacgagatattaatgtataacatgtgctttgttgttgtgggtcgtggttgacgcttaattacttttatgatcccacaatctatcaataagtcaacacttttgttgtttcttgtcaaatcaataagtcaaatagttatgaaataataaatgaactaaaagactacttgaaattttggatgggtgattcgtataaagagacacgtaatgaatgtttacccaataaactaaactatgcaaaaacaaaaaataatcatcacttagtactgttataaacctgtatatggatcttcttaaataattaaaatgtgtgaattcatttcataatccaaaaaatagatcattatataaagataaaaacaacacggcaaaaccaaccttaaagacaaagtggagaagctgtcctctaggcaatgggattaaagtgtttttgcagacattatcgttttatcgtgtgtgtcagtgtgtgtgacttaaactctatttaaaagcaaattattgtactatgtatttcacaaatgatgatatgtttgtttatctgtacgtctatgatatgtatctttcttatctttatgatatgatgcaaaatatggatgttagaagaacttttcatttttctttttctttttatgggattaaagttttttgttcgcactattttttcttttactataatatcttttcaattggaattcaaattattcacatataaaattctacaataaacaaacttaagtgaaattggaccaatcttataaaaccacacaactagaatcccttgttattcctttagttaagcatttgagataaatcttagcctaatataacgacctacctatatttcaattatttggctttaaatagcttggtaaattggacaaagcagaaatcgatttgatcgtctagctcaacgtgggtgctaccttggtttttaagattttagcttttaagataaaataatactattcatactggtgaaatgatttggattttgtctttggataaaatatactactgtattaatcgtttaccaaccacgagacactaaactttcacctaattgatcgacttgtatatactcaagataatgacaacgatatgtcatcaatgtctacatttgataaagccaaaatcttttagtttattaagtcaaatattggtactttaacatctcaattaccaataaatattcgaaatgtggtgtcacctaagatctagacaatgacattaatagttttgatgcctagatttggtgaaccactcatccatttttatttgatatttagaccacgaaattgttactcaagttactagacaataatagtacatagcgacaaatcttcacctaccgctagtattttctactaaactattcttgtagtatcttttactacttatacgaagatgacaaaaattagtaactctcaagtaccacatgaaacgtgtgtatgatcaaaactactctcaagttcgtctcagtagtcatgtttataggattctgaacgttttcactagagtccatatagtaatttgcattaaaactcatgaagaaaataacataagtaattatgaaagtacatagaaaattaagggaagtaaaactgacctttgatgcctatgtgacacgtaaacagtactctcaactgtccaatcgtaagcgttcctagccttccagggcccagcgtaagcaataccagccacaacaccctcaacctcagcaaccaaccaagggtatctatcttgcaacctctctagatcatcaatccactcttgtggtgtttgtggctctgtcctaaagttcactgtagacgtctcaatgtaatggttaacgatatcacaaaccgcggccatatcagctgctgtagctggcctaatctcaactggtctcctctccggagaagccatggttggatccttacctgttaatcagaaaaactcagattaatcgacaaattcgatcgcacaaactagaaactaacaccagatctagatagaaatcacaaatcgaagagtaattattcgacaaaactcaaattatttgaacaaatcggatgatatttatgaaaccctaatcgagaattaagatgatatctaacgatcaaacccagaaaatcgtcttcgatctaagattaacagaatctaaaccaaagaacatatacgaaattgggatcgaacgaaaacaaaatcgaagattttgagagaataaggaacacagaaatttaccttgatcacggtagagagaattgagagaaagtttttaagattttgagaaattgaaatctgaattgtgaagaagaagctttgggtattgttttatagaagaagaagaagaaaagacgaggacgactaggtcacgagaaagctaaggcggtgaagcaatagctaataataaaatgacacgtgtattgagcgttgtttacacgcaaagttgtttttggctaattgccttatttttaggttgaggaaaagtatttgtgctttgagttgataaacacgactcgtgtgtgccggctgcaaccactttgacgccgtttattactgactcgtcgacaaccacaatttctaacggtcgtcataagatccagccgttgagatttaacgatcgttacgatttatatttttttagcattatcgttttattttttaaatatacggtggagctgaaaattggcaataattgaaccgtgggtcccactgcattgaagcgtatttcgtattttctagaattcttcgtgctttatttcttttcctttttgtttttttttgccatttatctaatgcaagtgggcttataaaatcagtgaatttcttggaaaagtaacttctttatcgtataacatattgtgaaattatccatttcttttaattttttagtgttattggatatttttgtatgattattgatttgcataggataatgacttttgtatcaagttggtgaacaagtctcgttaaaaaaggcaagtggtttggtgactcgatttattcttgttatttaattcatatatcaatggatcttatttggggcctggtccatatttaacactcgtgttcagtccaatgaccaataatattttttcattaataacaatgtaacaagaatgatacacaaaacattctttgaataagttcgctatgaagaagggaacttatccggtcctagatcatcagttcatacaaacctccatagagttcaacatcttaaacaagaatatcctgatccgttgacctgcaggtcgacaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccaggggaatgcggccgggtttaaacatttaaatttaattaagcggccgcttgcccgggcatggcgcgccttaattaagcggtggccactattttcagaagaagttcccaatagtagtccaaaatttttgtaacgaagggagcataatagttacatgcaaaggaaaactgccattctttagaggggatgcttgtttaagaacaaaaaatatatcactttcttttgttccaagtcattgcgtatttttttaaaaatatttgttccttcgtatatttcgagcttcaatcactttatggttcattgtattctggctttgctgtaaatcgtagctaaccttcttcctagcagaaattattaatacttgggatatttttttagaatcaagtaaattacatattaccaccacatcgagctgcttttaaattcatattacagccatataggcttgattcattttgcaaaatttccaggatattgacaacgttaacttaataatatcttgaaatattaaagctattatgattaggggtgcaaatggaccgagttggttcggtttatatcaaaatcaaaccaaaccaactatatcggtttggattggttcggttttgccgggttttcagcattttctggttttttttttgttagatgaatattattttaatcttactttgtcaaatttttgataagtaaatatatgtgttagtaaaaattaattttttttacaaacatatgatctattaaaatattcttataggagaattttcttaataacacatgatatttatttattttagtcgtttgactaatttttcgttgatgtacactttcaaagttaaccaaatttagtaattaagtataaaaatcaatatgatacctaaataatgatatgttctatttaattttaaattatcgaaatttcacttcaaattcgaaaaagatatataagaattttgataggttttgacatatgaatatggaagaacaaagagattgacgcattttagtaacacttgataagaaagtgatcgtacaaccaattatttaaagttaataaaaatggagcacttcatatttaacgaaatattacatgccagaagagtcgcaaatatttctagatattttttaaagaaaattctataaaaagtcttaaaggcatatatataaaaactatatatttatattttggtttggttcgaatttgttttactcaataccaaactaaattagaccaaatataattgagatttttaatcgcggcccatgatcacaccggtccgagttcttgtacaccactacaagaactcggaggtgttctccgattcgctgcgagataccgagttcttgtacaccactacaagaactcggcacgggatacttgtagaggtacccacgccgagttcttgtacaccactacaagaactcggtactagtgctagccttgtcgacatttaaatgatgagtcggacccagctttcttgtacaaagtggttgcggccgcttaataagcttcttgcctcaattccggaggtgtttctagtgttcaacatgacaaacaaaacccatctctttcagtatatgtctctcagttgtgcttaattcaaatttcaactcagagaacttcttggcatacttatccagattatctaatgatctcatctaatggtaattcaactttcagtatatgtctcgcagcaaactatctttacatcaaatttttaacaactcaatgcacaaaatacttttcctcaacctaaaaataaggcaattagccaaaaacaactttgcgtgtgaacaacgcgttacacgtccctacacatacgtgtcaatttataattggctattgcttccacgccttagctttctcgtgaccgaccgagtcgtcctcgtcttttttgcttctataaatcaaatacccaaagagctcttcttcttcacaattcagattccaattttctcaaactctaaaatcaatctctcaaatctctcaaccgtgatcaaggtagatttctgagttcttattgtatttcttcgatttgtttcgttcgatcgcaatttaggctctgttctttgattttgatctcgttaatctctgatcggaggcaaattacatagtttcatcgttagatctcttcttatttctcgattagggttcgtatttttcgcagatctgtttattttcttgttgtttccttgtatttgatccgatttgttgaaagaatttgtgtgttctcgattatttacgctttgatctgtgatttttatctagatttggtgttagtttcttgtttgtgcgatcgaatttgtcgattaatctcggtttttctgattaacagggatccaaccatggatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagagctcggtaacctttaaactgagggcactgaagtcgcttgatgtgctgaattgtttgtgatgttggtggcgtattttgtttaaataagtaagcatggctgtgattttatcatatgatcgatctttggggttttatttaacacattgtaaaatgtgtatctattaataactcaatgtataagatgtgttcattcttcggttgccatagatctgcttatttgacctgtgatgttttgactccaaaaaccaaaatcacaactcaataaactcatggaatatgtccacctgtttcttgaagagttcatctaccattccagttggcatttatcagtgttgcagcggcgctgtgctttgtaacataacaattgttacggcatatatccaa

SEQ ID NO: 4 - DNA, 4148 bp, artificial sequence
  Gene expression cassette of pDAB118261
ccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtagatctccatggcccccaagaagaagaggaaggtgggcatccacggggtacccgccgctatggccgagagacccttccagtgccggatctgcatgcggaacttcagcaggagcgacgacctgagcaagcacatcagaacccacaccggcgagaagcccttcgcctgcgatatctgcggcaggaagttcgccgacaacagcaaccggatcaagcacaccaagatccacaccggcagccagaagcctttccagtgtcgcatctgtatgcgcaacttctcccggtctgatgccctgtccgtgcacatcaggacacacacaggggagaagccttttgcctgtgacatctgcggccgcaagtttgctgacaacgccaaccgcacaaagcacgcccagcgctgcggcggcctgcgcggatcccaacttgtgaaatcagaattggaagagaaaaagtctgagcttagacacaaattgaagtacgttccacatgaatatatcgaacttatcgagattgctaggaactcaacacaggacagaattttggagatgaaggttatggagttctttatgaaagtgtacggatataggggaaagcaccttggtggttctaggaaacctgatggtgcaatctacactgtgggatcacctattgactatggtgttatcgtggatacaaaggcatactctggtggatacaatttgccaatcggacaagctgacgaaatgcagagatatgttgaagagaaccaaactagaaacaaacatattaatccaaatgaatggtggaaggtgtatccttcatctgttacagagttcaaattcctttttgtgtctggacactttaagggtaactacaaagcacagcttactaggttgaaccatattacaaattgcaatggtgctgtgttgtcagttgaagagcttttgatcggaggtgaaatgattaaggcaggaacacttactttggaggaagttagaagaaaattcaacaacggtgaaatcaattttagatcttgataactcgagctcggtcaccagcataatttttattaatgtactaaattactgttttgttaaatgcaattttgctttctcgggattttaatatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggttgcggccgcaagggtgggcgcgccgacccagctttcttgtacaaagtggttgcggccgcttaataagcttcttgcctcaattccggaggtgtttctagtgttcaacatgacaaacaaaacccatctctttcagtatatgtctctcagttgtgcttaattcaaatttcaactcagagaacttcttggcatacttatccagattatctaatgatctcatctaatggtaattcaactttcagtatatgtctcgcagcaaactatctttacatcaaatttttaacaactcaatgcacaaaatacttttcctcaacctaaaaataaggcaattagccaaaaacaactttgcgtgtgaacaacgcgttacacgtccctacacatacgtgtcaatttataattggctattgcttccacgccttagctttctcgtgaccgaccgagtcgtcctcgtcttttttgcttctataaatcaaatacccaaagagctcttcttcttcacaattcagattccaattttctcaaactctaaaatcaatctctcaaatctctcaaccgtgatcaaggtagatttctgagttcttattgtatttcttcgatttgtttcgttcgatcgcaatttaggctctgttctttgattttgatctcgttaatctctgatcggaggcaaattacatagtttcatcgttagatctcttcttatttctcgattagggttcgtatttttcgcagatctgtttattttcttgttgtttccttgtatttgatccgatttgttgaaagaatttgtgtgttctcgattatttacgctttgatctgtgatttttatctagatttggtgttagtttcttgtttgtgcgatcgaatttgtcgattaatctcggtttttctgattaacagggatccaaccatggatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagagctcggtaacctttaaactgagggcactgaagtcgcttgatgtgctgaattgtttgtgatgttggtggcgtattttgtttaaataagtaagcatggctgtgattttatcatatgatcgatctttggggttttatttaacacattgtaaaatgtgtatctattaataactcaatgtataagatgtgttcattcttcggttgccatagatctgcttatttgacctgtgatgttttgactccaaaaaccaaaatcacaactcaataaactcatggaatatgtccacctgtttcttgaagagttcatctaccattccagttggcatttatcagtgttgcagcggcgctgtgctttgtaacataacaattgttacggcatatatccaa

SEQ ID NO: 5 - DNA, 29 bp, artificial sequence
  primer sequence
acaagagtggattgatgatctagagaggt

SEQ ID NO: 6 - DNA, 29 bp, artificial sequence
  primer sequence
ctttgatgcctatgtgacacgtaaacagt

SEQ ID NO: 7 - DNA, 29 bp, artificial sequence
  primer sequence
ggtgttgtggctggtattgcttacgctgg

SEQ ID NO: 8 - DNA, 17 bp, artificial sequence
  primer sequence
acgacgggcgttccttg

SEQ ID NO: 9 - DNA, 24 bp, artificial sequence
  primer sequence
gagcaaggtgagatgacaggagat

SEQ ID NO: 10 - DNA, 23 bp, artificial sequence
  primer sequence
cactgaagcgggaagggactggc

SEQ ID NO: 11 - DNA, 30 bp, artificial sequence
  primer sequence
tactatgacttgatgttgtgtggtgactga

SEQ ID NO: 12 - DNA, 28 bp, artificial sequence
  primer sequence
gagcggtctaaattccgacccttatttc

SEQ ID NO: 13 - DNA, 33 bp, artificial sequence
  primer sequence
aaacgatggcaggagtgccctttttctatcaat

SEQ ID NO: 14 - DNA, 21 bp, artificial sequence
  primer sequence
tgaatggtggaaggtgtatcc

SEQ ID NO: 15 - DNA, 25 bp, artificial sequence
  primer sequence
aagctgtgctttgtagttaccctta

SEQ ID NO: 16 - DNA, 30 bp, artificial sequence
  Site specific nuclease binding site (IL-1)
attatccgagttcaccagaactcggataat

SEQ ID NO: 17 - DNA, 21 bp, artificial sequence
  primer sequence
ccatggttgttgagaccttct

SEQ ID NO: 18 - DNA, 20 bp, artificial sequence
  primer sequence
gcatgtccctcacagcaaaa

SEQ ID NO: 19 - DNA, 17 bp, artificial sequence
  primer sequence
agtacccaccattggga

SEQ ID NO: 20 - DNA, 20 bp, artificial sequence
  primer sequence
tgaactttaggacagagcca

SEQ ID NO: 21 - DNA, 19 bp, artificial sequence
  primer sequence
tgtgtatcccaaagcctca

SEQ ID NO: 22 - DNA, 21 bp, artificial sequence
  primer sequence
gcctggtccatatttaacact

SEQ ID NO: 23 - DNA, 20 bp, artificial sequence
  primer sequence
ttgggctgaattgaagacat

SEQ ID NO: 24 - DNA, 7743 bp, artificial sequence
  Gene expression cassette from pDAB118253
gatgtgaagaacaggtaaatcacgcagaagaacccatctctgatagcagctatcgattagaacaacgaatccatattgggtccgtgggaaatacttactgcacaggaagggggcgatctgacgaggccccgccaccggcctcgacccgaggccgaggccgacgaagcgccggcgagtacggcgccgcggcggcctctgcccgtgccctctgcgcgtgggagggagaggccgcggtggtgggggcgcgcgcgcgcgcgcgcgcagctggtgcggcggcgcgggggtcagccgccgagccggcggcgacggaggagcagggcggcgtggacgcgaacttccgatcggttggtcagagtgcgcgagttgggcttagccaattaggtctcaacaatctattgggccgtaaaattcatgggccctggtttgtctaggcccaatatcccgttcatttcagcccacaaatatttccccagaggattattaaggcccacacgcagcttatagcagatcaagtacgatgtttcctgatcgttggatcggaaacgtacggtcttgatcaggcatgccgacttcgtcaaagagaggcggcatgacctgacgcggagttggttccgggcaccgtctggatggtcgtaccgggaccggacacgtgtcgcgcctccaactacatggacacgtgtggtgctgccattgggccgtacgcgtggcggtgaccgcaccggatgctgcctcgcaccgccttgcccacgctttatatagagaggttttctctccattaatcgcatagcgagtcgaatcgaccgaaggggagggggagcgaagctttgcgttctctaatcgcctcgtcaaggtaactaatcaatcacctcgtcctaatcctcgaatctctcgtggtgcccgtctaatctcgcgattttgatgctcgtggtggaaagcgtaggaggatcccgtgcgagttagtctcaatctctcagggtttcgtgcgattttagggtgatccacctcttaatcgagttacggtttcgtgcgattttagggtaatcctcttaatctctcattgatttagggtttcgtgagaatcgaggtagggatctgtgttatttatatcgatctaatagatggattggttttgagattgttctgtcagatggggattgtttcgatatattaccctaatgatgtgtcagatggggattgtttcgatatattaccctaatgatgtgtcagatggggattgtttcgatatattaccctaatgatggataataagagtagttcacagttatgttttgatcctgccacatagtttgagttttgtgatcagatttagtttcacttatttgtgcttagttcggatgggattgttctgatattgttccaatagatgaatagctcgttaggttaaaatctttaggttgagttaggcgacacatagtttatttcctctggatttggattggaattgtgttcttagtttttttcccctggatttggattggaattgtgtggagctgggttagagaattacatctgtatcgtgtacacctacttgaactgtagagcttgggttctaaggtcaatttaatctgtattgtatctggctctttgcctagttgaactgtagtgctgatgttgtactgtgtttttttacccgttttatttgctttactcgtgcaaatcaaatctgtcagatgctagaactaggtggctttattctgtgttcttacatagatctgttgtcctgtagttacttatgtcagttttgttattatctgaagatatttttggttgttgcttgttgatgtggtgtgagctgtgagcagcgctcttatgattaatgatgctgtccaattgtagtgtaatatgatgtgattgatatgttcatctattttgagctgacagtaccgatatcgtaggatctggtgccaacttattctccagctgcttttttttacctatgttaattccaatcctttcttgcctcttccagatccagatacaatcctgtccctagtggataaactgcaaaaggcccacacgacaccatgtcatctggagcacttctctttcatgggaagattccttacgttgtggagatggaagggaatgttgatggccacacctttagcatacgtgggaaaggctacggagatgcctcagtgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatgcacaattcatctgtactaccggagatgttcctgtgccttggagcacacttgtcaccactctcacctatggagcacagtgctttgccaagtatggtccagagttgaaggacttctacaagtcctgtatgccagatggctatgtgcaagagcgcacaatcacctttgaaggagatggcaacttcaagactagggctgaagtcacctttgagaatgggtctgtctacaatagggtcaaactcaatggtcaaggcttcaagaaagatggtcacgtgttgggaaagaacttggagttcaacttcactccccactgcctctacatctggggagaccaagccaaccacggtctcaagtcagccttcaagatatgtcatgagattactggcagcaaaggcgacttcatagtggctgaccacacccagatgaacactcccattggtggaggtccagttcatgttccagagtatcatcatatgtcttaccatgtgaaactttccaaagatgtgacagaccacagagacaacatgagcttgaaagaaactgtcagagctgttgactgtcgcaagacctacctttgagtagttagcttaatcacctagagctcggtaacctttaaactgagggcactgaagtcgcttgatgtgctgaattgtttgtgatgttggtggcgtattttgtttaaataagtaagcatggctgtgattttatcatatgatcgatctttggggttttatttaacacattgtaaaatgtgtatctattaataactcaatgtataagatgtgttcattcttcggttgccatagatctgcttatttgacctgtgatgttttgactccaaaaaccaaaatcacaactcaataaactcatggaatatgtccacctgtttcttgaagagttcatctaccattccagttggcatttatcagtgttgcagcggcgctgtgctttgtaacataacaattgttacggcatatatccaacaatcctgtccctagtggataaactgcaaaaggcccagatctggttaatgagggaaagtaatctacagaagtacagtcccctctgaaaggaacatggcttatactccatgccttcaagtagatttgaagtcattgcttcacttaggaagttctattgtttgagatggtggttatgacaggctccgtttaaacttgctggtgttatgtgcccttgaagtagccaggaggtttgttgttcccatacacccatgagggcttggtcagttttctctggtatgttactggatggagagggtctccaagtgtgcaatgtgaacttaacaacatcccagggcactcacccctcactgacttttttggctactaataaacagactagctatcattcacctttgtagccagaatcccaggggatctgcacccagtactcattccagcttgcagaataatatccaccaggtcatgtaataagaggagtcaaggacttagtcttgtttcctcactcatcaattatgaggtattgccatgatgcctagccacctcacacaacctagacaaggccactgtgtccaagactcattcacccagtcactagcacccagtcttgcacccaggggggttctggaggccctccaaatactaagtaacatttttccaggcacacaatgtgtgagtaggacacagtgcagttgatcaaacccaggtttcttgtgtagtgccttccctattgaggaccagggagcatggccacctctcaaagtcagctctcatcaatctctcaacaaactgtttacatattatctctgattcccaggaggactcagccccacaacttcccatttcaggtatcccttggcatcctagccctaatgcccatttgttaatatgggatggctcacctctgaggactctagttacagggtggagtctcccttgggatttcagttggtaggttaaaaatgggagtggcatggagaggaaataacagaggccctccagcaatagcttctctgtgatgataacccctatactctatattttaatctagacccagctttcttgtacaaagtggttgcggccgcttaattaaatttaaatccaagcttgggctgcagatccccggggatctccgcggagtatcggaagttgaagacaaagaaggtcttaaatcctggctagcaacactgaactatgccagaaaccacatcaaagcatatcggcaagcttcttggcccattatatccaaagacctcagagaaaggtgagcgaaggctcaattcagaagattggaagctgatcaataggatcaagacaatggtgagaacgcttccaaatctcactattccaccagaagatgcatacattatcattgaaacagatgcatgtgcaactggatggggagcagtatgcaagtggaagaaaaacaaggcagacccaagaaatacagagcaaatctgtaggtatgccagtggaaaatttgataagccaaaaggaacctgtgatgcagaaatctatggggttatgaatggcttagaaaagatgagattgttctacttggacaaaagagagatcacagtcagaactgacagtagtgcaatcgaaaggttctacaacaagagtgctgaacacaagccttctgagatcagatggatcaggttcatggactacatcactggtgcaggaccagagatagtcattgaacacataaaagggaagagcaatggtttagctgacatcttgtccaggctcaaagccaaattagctcagaatgaaccaacggaagagatgatcctgcttacacaagccataagggaagtaattccttatccagatcatccatacactgagcaactcagagaatggggaaacaaaattctggatccattccccacattcaagaaggacatgttcgaaagaacagagcaagcttttatgctaacagaggaaccagttctactctgtgcatgcaggaagcctgcaattcagttagtgtccagaacatctgccaacccaggaaggaaattcttcaagtgcgcaatgaacaaatgccattgctggtactgggcagatctcattgaagaacacattcaagacagaattgatgaatttctcaagaatcttgaagttctgaagaccggtggcgtgcaaacaatggaggaggaacttatgaaggaagtcaccaagctgaagatagaagagcaggagttcgaggaataccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcaccaaggcccggggtcggatctagctgaaggctcgacaaggcagtccacggaggagctgatatttggtggacaagctgtggataggagcaaccctatccctaatataccagcaccaccaagtcagggcaatccccagatcaccccagcagattcgaagaaggtacagtacacacacatgtatatatgtatgatgtatcccttcgatcgaaggcatgccttggtataatcactgagtagtcattttattactttgttttgacaagtcagtagttcatccatttgtcccattttttcagcttggaagtttggttgcactggccttggtctaataactgagtagtcattttattacgttgtttcgacaagtcagtagctcatccatctgtcccattttttcagctaggaagtttggttgcactggccttggactaataactgattagtcattttattacattgtttcgacaagtcagtagctcatccatctgtcccatttttcagctaggaagttcggatctggggccatttgttccaggcacgggataagcattcagggatccaaccatggctcatgctgccctcagccctctctcccaacgctttgagagaatagctgtccagccactcactggtgtccttggtgctgagatcactggagtggacttgagggaaccacttgatgacagcacctggaatgagatattggatgccttccacacttaccaagtcatctactttcctggccaagcaatcaccaatgagcagcacattgcattctcaagaaggtttggaccagttgatccagtgcctcttctcaagagcattgaaggctatccagaggttcagatgatccgcagagaagccaatgagtctggaagggtgattggtgatgactggcacacagactccactttccttgatgcacctccagctgctgttgtgatgagggccatagatgttcctgagcatggcggagacactgggttcctttcaatgtacacagcttgggagaccttgtctccaaccatgcaagccaccatcgaagggctcaacgttgtgcactctgccacacgtgtgttcggttccctctaccaagcacagaaccgtcgcttcagcaacacctcagtcaaggtgatggatgttgatgctggtgacagagagacagtccatcccttggttgtgactcatcctggctctggaaggaaaggcctttatgtgaatcaagtctactgtcagagaattgagggcatgacagatgcagaatcaaagccattgcttcagttcctctatgagcatgccaccagatttgacttcacttgccgtgtgaggtggaagaaagaccaagtccttgtctgggacaacttgtgcaccatgcaccgtgctgttcctgactatgctggcaagttcagatacttgactcgcaccacagttggtggagttaggcctgcccgctgagtagttagcttaatcacctagagctcggtcgcagcgtgtgcgtgtccgtcgtacgttctggccggccgggccttgggcgcgcgatcagaagcgttgcgttggcgtgtgtgtgcttctggtttgctttaattttaccaagtttgtttcaaggtggatcgcgtggtcaaggcccgtgtgctttaaagacccaccggcactggcagtgagtgttgctgcttgtgtaggctttggtacgtatgggctttatttgcttctggatgttgtgtactacttgggtttgttgaattattatgagcagttgcgtattgtaattcagctgggctacctggacattgttatgtattaataaatgctttgctttcttctaaagatctttaagtgct

SEQ ID NO: 25 - DNA, 5103 bp, artificial sequence
  Gene expression cassette from pDAB118254
caatcctgtccctagtggataaactgcaaaaggctcaactaactaaagcttccacacgacaccatgttggctcgccaaggaggatcactgagagcctctcagtgtaacgctggcctcgcgagacgcgtggaggtgggagcgttggttgttccgagacccataagcgtcaacgacgtggttccccatgtctattcggctcctctgagcgtcgcgaggaggtcgtgctccaagtcatccatccgctcgactcgcagacttcagacaaccgtctgctccgcaagagggatgccagccttgtcgctgcctggctcaaagtcgatcacggctagagcactctttctcgcagcagcagccgacggagtcaccacgcttgtgagaccgctgcggtcagacgacaccgagggttttgcggaaggcctcgtcagactgggctatcgggttgggaggactcccgacacgtggcaagtggacggaaggccacaaggtccagcagttgccgaggctgatgtgtattgtagagacggtgcaacaacggctaggttcctccccacactcgcagctgctggacacgggacctacagatttgatgcctctccccagatgaggagaaggccactgctgcctctttctagggctttgagggaccttggcgttgatcttcgccacgaggaagcggaagggcaccaccccttgaccgtgagagctgctggagtcgagggaggtgaggttacactcgatgctggacagtcctctcagtacttgacggcactgctgctgctcggtccgctcacacgccaagggctgcggattcgcgtcactgatctggttagcgctccgtacgtggagattacacttgcgatgatgagagcttttggggtcgaggttgcacgcgaaggcgacgttttcgtggtgcctcctggtggctacagagcgactacgtacgcgattgagccagatgccagcaccgcaagctacttctttgcagctgctgcgttgacacctggagccgaggtcacagtgcctggactcgggaccggagcgcttcaaggggatctcggcttcgtggacgtgctgcggaggatgggtgccgaggtcagcgtgggagcagacgctacgactgttagaggcacgggtgagcttagaggccttacagcaaacatgagggacatatccgacacgatgccgacgcttgctgccatcgctccgttcgcttcagcacccgtcagaattgaagatgtggcgaacactcgcgtcaaagagtgcgacagacttgaagcgtgtgccgagaacttgaggaggttgggagtgagagtcgcaactggtccagactggatcgagatccaccctggtccagctactggagcgcaagtcacaagctatggcgaccataggattgttatgtcattcgcagtgaccggactcagagttcctgggatctctttcgacgaccctggttgcgtgcggaaaacgttccctggcttccacgaggcatttgcggagctgcggagaggaattggttcctgagtagttagcttaatcacctagagctcggtcgcagcgtgtgcgtgtccgtcgtacgttctggccggccgggccttgggcgcgcgatcagaagcgttgcgttggcgtgtgtgtgcttctggtttgctttaattttaccaagtttgtttcaaggtggatcgcgtggtcaaggcccgtgtgctttaaagacccaccggcactggcagtgagtgttgctgcttgtgtaggctttggtacgtatgggctttatttgcttctggatgttgtgtactacttgggtttgttgaattattatgagcagttgcgtattgtaattcagctgggctacctggacattgttatgtattaataaatgctttgctttcttctaaagatctttaagtgctgcggccgcttaattaaccttgatatctaggcccagcttgctcttctgtagtttaaacttagttgattgacaatcctgtccctagtggataaactgcaaaaggctactagtgctagcctctcgagttgtcgacatttaaatgatgagtcggacccagctttcttgtacaaagtggttgcggccgcttaattaaatttaaatgtttggggatcctctagagtcgacctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacagtagttagttgacacgacaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccgtggtacattgcgcgcagctggatacaagcatggtggatggcatgatgttggtttttggcaaagggattttgagttgccagctcctccaaggccagttaggccagttacccagatctgactgagcttgagcttatgagcttatgagcttagagctcggtcgcagcgtgtgcgtgtccgtcgtacgttctggccggccgggccttgggcgcgcgatcagaagcgttgcgttggcgtgtgtgtgcttctggtttgctttaattttaccaagtttgtttcaaggtggatcgcgtggtcaaggcccgtgtgctttaaagacccaccggcactggcagtgagtgttgctgcttgtgtaggctttggtacgtatgggctttatttgcttctggatgttgtgtactacttgggtttgttgaattattatgagcagttgcgtattgtaattcagctgggctacctggacattgttatgtattaataaatgctttgctttcttctaaagatctttaagtgct

SEQ ID NO: 26 - DNA, 7256 bp, artificial sequence
  Gene expression cassette from pDAB113068
acaaattcggcgccatgcccgggcaagcggccgcacaagtttgtacaaaaaagcaggctcaatcctgtccctagtggataaactgcaaaaggcgtaactaatcaatcacctcgtcctaatcctcgaatctctcgtggtgcccgtctaatctcgcgattttgatgctcgtggtggaaagcgtaggaggatcccgtgcgagttagtctcaatctctcagggtttcgtgcgattttagggtgatccacctcttaatcgagttacggtttcgtgcgattttagggtaatcctcttaatctctcattgatttagggtttcgtgagaatcgaggtagggatctgtgttatttatatcgatctaatagatggattggttttgagattgttctgtcagatggggattgtttcgatatattaccctaatgatgtgtcagatggggattgtttcgatatattaccctaatgatgtgtcagatggggattgtttcgatatattaccctaatgatggataataagagtagttcacagttatgttttgatcctgccacatagtttgagttttgtgatcagatttagtttcacttatttgtgcttagttcggatgggattgttctgatattgttccaatagatgaatagctcgttaggttaaaatctttaggttgagttaggcgacacatagtttatttcctctggatttggattggaattgtgttcttagtttttttcccctggatttggattggaattgtgtggagctgggttagagaattacatctgtatcgtgtacacctacttgaactgtagagcttgggttctaaggtcaatttaatctgtattgtatctggctctttgcctagttgaactgtagtgctgatgttgtactgtgtttttttacccgttttatttgctttactcgtgcaaatcaaatctgtcagatgctagaactaggtggctttattctgtgttcttacatagatctgttgtcctgtagttacttatgtcagttttgttattatctgaagatatttttggttgttgcttgttgatgtggtgtgagctgtgagcagcgctcttatgattaatgatgctgtccaattgtagtgtaatatgatgtgattgatatgttcatctattttgagctgacagtaccgatatcgtaggatctggtgccaacttattctccagctgcttttttttacctatgttaattccaatcctttcttgcctcttccagatccagataccacacgacaccatgttggctcgccaaggaggatcactgagagcctctcagtgtaacgctggcctcgcgagacgcgtggaggtgggagcgttggttgttccgagacccataagcgtcaacgacgtggttccccatgtctattcggctcctctgagcgtcgcgaggaggtcgtgctccaagtcatccatccgctcgactcgcagacttcagacaaccgtctgctccgcaagagggatgccagccttgtcgctgcctggctcaaagtcgatcacggctagagcactctttctcgcagcagcagccgacggagtcaccacgcttgtgagaccgctgcggtcagacgacaccgagggttttgcggaaggcctcgtcagactgggctatcgggttgggaggactcccgacacgtggcaagtggacggaaggccacaaggtccagcagttgccgaggctgatgtgtattgtagagacggtgcaacaacggctaggttcctccccacactcgcagctgctggacacgggacctacagatttgatgcctctccccagatgaggagaaggccactgctgcctctttctagggctttgagggaccttggcgttgatcttcgccacgaggaagcggaagggcaccaccccttgaccgtgagagctgctggagtcgagggaggtgaggttacactcgatgctggacagtcctctcagtacttgacggcactgctgctgctcggtccgctcacacgccaagggctgcggattcgcgtcactgatctggttagcgctccgtacgtggagattacacttgcgatgatgagagcttttggggtcgaggttgcacgcgaaggcgacgttttcgtggtgcctcctggtggctacagagcgactacgtacgcgattgagccagatgccagcaccgcaagctacttctttgcagctgctgcgttgacacctggagccgaggtcacagtgcctggactcgggaccggagcgcttcaaggggatctcggcttcgtggacgtgctgcggaggatgggtgccgaggtcagcgtgggagcagacgctacgactgttagaggcacgggtgagcttagaggccttacagcaaacatgagggacatatccgacacgatgccgacgcttgctgccatcgctccgttcgcttcagcacccgtcagaattgaagatgtggcgaacactcgcgtcaaagagtgcgacagacttgaagcgtgtgccgagaacttgaggaggttgggagtgagagtcgcaactggtccagactggatcgagatccaccctggtccagctactggagcgcaagtcacaagctatggcgaccataggattgttatgtcattcgcagtgaccggactcagagttcctgggatctctttcgacgaccctggttgcgtgcggaaaacgttccctggcttccacgaggcatttgcggagctgcggagaggaattggttcctgagtagttagcttaatcacctagagctcggtcgcagcgtgtgcgtgtccgtcgtacgttctggccggccgggccttgggcgcgcgatcagaagcgttgcgttggcgtgtgtgtgcttctggtttgctttaattttaccaagtttgtttcaaggtggatcgcgtggtcaaggcccgtgtgctttaaagacccaccggcactggcagtgagtgttgctgcttgtgtaggctttggtacgtatgggctttatttgcttctggatgttgtgtactacttgggtttgttgaattattatgagcagttgcgtattgtaattcagctgggctacctggacattgttatgtattaataaatgctttgctttcttctaaagatctttaagtgctgccttttgcagtttatctctatgcccgggacaagtgcaatcctgtccctagtgagatgggcgggagtcttccagatctggttaatgagggaaagtaatctacagaagtacagtcccctctgaaaggaacatggcttatactccatgccttcaagtagatttgaagtcattgcttcacttaggaagttctattgtttgagatggtggttatgacaggctccgtttaaacttgctggtgttatgtgcccttgaagtagccaggaggtttgttgttcccatacacccatgagggcttggtcagttttctctggtatgttactggatggagagggtctccaagtgtgcaatgtgaacttaacaacatcccagggcactcacccctcactgacttttttggctactaataaacagactagctatcattcacctttgtagccagaatcccaggggatctgcacccagtactcattccagcttgcagaataatatccaccaggtcatgtaataagaggagtcaaggacttagtcttgtttcctcactcatcaattatgaggtattgccatgatgcctagccacctcacacaacctagacaaggccactgtgtccaagactcattcacccagtcactagcacccagtcttgcacccaggggggttctggaggccctccaaatactaagtaacatttttccaggcacacaatgtgtgagtaggacacagtgcagttgatcaaacccaggtttcttgtgtagtgccttccctattgaggaccagggagcatggccacctctcaaagtcagctctcatcaatctctcaacaaactgtttacatattatctctgattcccaggaggactcagccccacaacttcccatttcaggtatcccttggcatcctagccctaatgcccatttgttaatatgggatggctcacctctgaggactctagttacagggtggagtctcccttgggatttcagttggtaggttaaaaatgggagtggcatggagaggaaataacagaggccctccagcaatagcttctctgtgatgataacccctatactctatattttacaatcctgtccctagtggataaactgcaaaaggcacccagctttcttgtacaaagtggttgcggccgcttaattaaatttaaatgtttggggatcctctagagtcgacctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacagtagttagttgacacgacaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccgtggtacattgcgcgcagctggatacaagcatggtggatggcatgatgttggtttttggcaaagggattttgagttgccagctcctccaaggccagttaggccagttacccagatctgactgagcttgagcttatgagcttatgagcttagagctcggtcgcagcgtgtgcgtgtccgtcgtacgttctggccggccgggccttgggcgcgcgatcagaagcgttgcgttggcgtgtgtgtgcttctggtttgctttaattttaccaagtttgtttcaaggtggatcgcgtggtcaaggcccgtgtgctttaaagacccaccggcactggcagtgagtgttgctgcttgtgtaggctttggtacgtatgggctttatttgcttctggatgttgtgtactacttgggtttgttgaattattatgagcagttgcgtattgtaattcagctgggctacctggacattgttatgtattaataaatgctttgctttcttctaaagatctttaagtgct

SEQ ID NO: 27 - DNA, 7746 bp, artificial sequence
  Gene expression cassette from pDAB118280
aattacaacggtatatatcctgccagtcagcatcatcacaccaaaagttaggcccgaatagtttgaaattagaaagctcgcaattgaggtctacaggccaaattcgctcttagccgtacaatattactcaccagatcctaaccggtgtgatcatgggccgcgattaaaaatctcaattatatttggtctaatttagtttggtattgagtaaaacaaattcggcgccatgcccgggcaagcggccgcacaagtttgtacaaaaaagcaggctgagtattcactctagacctaggtagcaatcctgtccctagtggataaactgcaaaaggctcaactaactaaagcttgtaactaatcaatcacctcgtcctaatcctcgaatctctcgtggtgcccgtctaatctcgcgattttgatgctcgtggtggaaagcgtaggaggatcccgtgcgagttagtctcaatctctcagggtttcgtgcgattttagggtgatccacctcttaatcgagttacggtttcgtgcgattttagggtaatcctcttaatctctcattgatttagggtttcgtgagaatcgaggtagggatctgtgttatttatatcgatctaatagatggattggttttgagattgttctgtcagatggggattgtttcgatatattaccctaatgatgtgtcagatggggattgtttcgatatattaccctaatgatgtgtcagatggggattgtttcgatatattaccctaatgatggataataagagtagttcacagttatgttttgatcctgccacatagtttgagttttgtgatcagatttagtttcacttatttgtgcttagttcggatgggattgttctgatattgttccaatagatgaatagctcgttaggttaaaatctttaggttgagttaggcgacacatagtttatttcctctggatttggattggaattgtgttcttagtttttttcccctggatttggattggaattgtgtggagctgggttagagaattacatctgtatcgtgtacacctacttgaactgtagagcttgggttctaaggtcaatttaatctgtattgtatctggctctttgcctagttgaactgtagtgctgatgttgtactgtgtttttttacccgttttatttgctttactcgtgcaaatcaaatctgtcagatgctagaactaggtggctttattctgtgttcttacatagatctgttgtcctgtagttacttatgtcagttttgttattatctgaagatatttttggttgttgcttgttgatgtggtgtgagctgtgagcagcgctcttatgattaatgatgctgtccaattgtagtgtaatatgatgtgattgatatgttcatctattttgagctgacagtaccgatatcgtaggatctggtgccaacttattctccagctgcttttttttacctatgttaattccaatcctttcttgcctcttccagatccagataccacacgacaccatgttggctcgccaaggaggatcactgagagcctctcagtgtaacgctggcctcgcgagacgcgtggaggtgggagcgttggttgttccgagacccataagcgtcaacgacgtggttccccatgtctattcggctcctctgagcgtcgcgaggaggtcgtgctccaagtcatccatccgctcgactcgcagacttcagacaaccgtctgctccgcaagagggatgccagccttgtcgctgcctggctcaaagtcgatcacggctagagcactctttctcgcagcagcagccgacggagtcaccacgcttgtgagaccgctgcggtcagacgacaccgagggttttgcggaaggcctcgtcagactgggctatcgggttgggaggactcccgacacgtggcaagtggacggaaggccacaaggtccagcagttgccgaggctgatgtgtattgtagagacggtgcaacaacggctaggttcctccccacactcgcagctgctggacacgggacctacagatttgatgcctctccccagatgaggagaaggccactgctgcctctttctagggctttgagggaccttggcgttgatcttcgccacgaggaagcggaagggcaccaccccttgaccgtgagagctgctggagtcgagggaggtgaggttacactcgatgctggacagtcctctcagtacttgacggcactgctgctgctcggtccgctcacacgccaagggctgcggattcgcgtcactgatctggttagcgctccgtacgtggagattacacttgcgatgatgagagcttttggggtcgaggttgcacgcgaaggcgacgttttcgtggtgcctcctggtggctacagagcgactacgtacgcgattgagccagatgccagcaccgcaagctacttctttgcagctgctgcgttgacacctggagccgaggtcacagtgcctggactcgggaccggagcgcttcaaggggatctcggcttcgtggacgtgctgcggaggatgggtgccgaggtcagcgtgggagcagacgctacgactgttagaggcacgggtgagcttagaggccttacagcaaacatgagggacatatccgacacgatgccgacgcttgctgccatcgctccgttcgcttcagcacccgtcagaattgaagatgtggcgaacactcgcgtcaaagagtgcgacagacttgaagcgtgtgccgagaacttgaggaggttgggagtgagagtcgcaactggtccagactggatcgagatccaccctggtccagctactggagcgcaagtcacaagctatggcgaccataggattgttatgtcattcgcagtgaccggactcagagttcctgggatctctttcgacgaccctggttgcgtgcggaaaacgttccctggcttccacgaggcatttgcggagctgcggagaggaattggttcctgagtagttagcttaatcacctagagctcggtcgcagcgtgtgcgtgtccgtcgtacgttctggccggccgggccttgggcgcgcgatcagaagcgttgcgttggcgtgtgtgtgcttctggtttgctttaattttaccaagtttgtttcaaggtggatcgcgtggtcaaggcccgtgtgctttaaagacccaccggcactggcagtgagtgttgctgcttgtgtaggctttggtacgtatgggctttatttgcttctggatgttgtgtactacttgggtttgttgaattattatgagcagttgcgtattgtaattcagctgggctacctggacattgttatgtattaataaatgctttgctttcttctaaagatctttaagtgctgcggccgcgcccatcggtcatggatgcttctactgtacctgggtcgtctggtctctgcctgtgtcacctttgaagtacctgtgtcgggattgtgtttggtcatgaactgcagtttgtctttgatgttcttttgtctggtcttatgaactggttgtatctgtatgtttactgtaaactgttgttgcggtgcagcagtatggcatccgaatgaataaatgatgtttggacttaaatctgtactctgtttgttttcggttatgccagttctatattgcctgagatcagaatgtttagcttttgagttctgtttggcttgtggtcgactcctgtttcttacttgaggcgtaactctgttctggcaaactcaaatgtctaactgaatgttttaggacttaattgttggacagattaacgtgtttggtttgtttctagattgtgattcggaaggcttgttagttgtggaatcaaggagagcagctaggtctgtgcagaacgttattttggatttaagccttctcagattatgccattactctaaacctaatgatatcatatttcactcggggatgttggagtagtcttttctttctcctgcagacaaaatgattttgctttcgtgtgtgtacatgattttgtgcaactgttgcaacaactgaagtagacaagttttgacctcaccagaagaatgaaaaagattttggaatttgttacatcgacaaaccattgtaacttggcccatcagaatgcacagaagagcggctacaaattgacatgcgttgcaaactttgcaatagttgatgcacatgtttgccattgcctgccagtcttaggaaaagtgtgtggttcgagaaatctaagcatatgtgctctgctcacattgcgtggaacccacacagctttgtcacactcttgtccactccagaagtcattcctggcgctgtttacccctggtaaaaggtaaccgaaaacttctcaaggctgtacccaaaactggaaggaaatttggaggaaatctttgcttttgatcggctcactctttcgtttaaacgtcagaaacttagttgattgacaatcctgtccctagtggataaactgcaaaaggctactagtgctagcctctcgagttgtcgacatttaaatgatgagtcggacttgtacaaagtggttgcggccgcttaattaaatttaaatgtttggggatcctctagagtcgacctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcagggtacagtagttagttgacacgacaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccgtggtacattgcgcgcagctggatacaagcatggtggatggcatgatgttggtttttggcaaagggattttgagttgccagctcctccaaggccagttaggccagttacccagatctgactgagcttgagcttatgagcttatgagcttagagctcggtcgcagcgtgtgcgtgtccgtcgtacgttctggccggccgggccttgggcgcgcgatcagaagcgttgcgttggcgtgtgtgtgcttctggtttgctttaattttaccaagtttgtttcaaggtggatcgcgtggtcaaggcccgtgtgctttaaagacccaccggcactggcagtgagtgttgctgcttgtgtaggctttggtacgtatgggctttatttgcttctggatgttgtgtactacttgggtttgttgaattattatgagcagttgcgtattgtaattcagctgggctacctggacattgttatgtattaataaatgctttgctttcttctaaagatctttaagtgcttctagagcatgcacatagacacacacatcatctcattgatgcttggtaataattgtcattagattgtttttatgcatagatgcactcgaaatcagccaattttagacaagtatcaaacggatgtgacttcagtacattaaaaacgtccgcaatgtgttattaagttgtctaagcgtcaatttgatttacaattgaatatatcctgcc

SEQ ID NO: 28 - DNA, 12 bp, artificial sequence
  Site specific nuclease binding site for Scd27
gctcaagaacat

SEQ ID NO: 29 - DNA, 12 bp, artificial sequence
  Site specific nuclease binding site for scd27
gctcaagaacat

SEQ ID NO: 30 - DNA, 30 bp, artificial sequence
  Site specific nuclease binding site (IL-1)
attatccgagttctggtgaactcggataat

SEQ ID NO: 31 - DNA, 34 bp, artificial sequence
  Site specific binding site (eZFN1)
caatcctgtccctagtggataaactgcaaaaggc

SEQ ID NO: 32 - DNA, 34 bp, artificial sequence
  Site specific binding site (eZFN1)
gccttttgcagtttatccactagggacaggattg

SEQ ID NO: 33 - DNA, 14 bp, artificial sequence
  Site specific nuclease binding site (SBS8196)
gccttttgcagttt

SEQ ID NO: 34 - DNA, 14 bp, artificial sequence
  Site specific nuclease binding site (SBS8196)
aaactgcaaaaggc

SEQ ID NO: 35 - DNA, 17 bp, artificial sequence
  Site specific nuclease binding site (SBS19354)
tatgcccgggacaagtg

SEQ ID NO: 36 - DNA, 17 bp, Artificial sequence
  Site specific nuclease binding site (SBS19354)
cacttgtcccgggcata

SEQ ID NO: 37 - DNA, 14 bp, artificial sequence
  Site specific nuclease binding site (SBS15590)
caatcctgtcccta

SEQ ID NO: 38 - DNA, 14 bp, artificial sequence
  site specific nuclease binding site (SBS15590)
tagggacaggattg

SEQ ID NO: 39 - DNA, 34 bp, artificial sequence
  Site specific nuclease binding site (eZFN8)
caatcctgtccctagtgagatgggcgggagtctt

SEQ ID NO: 40 - DNA, 34 bp, artificial sequence
  Site specific nuclease binding site (eZFN8)
aagactcccgcccatctcactagggacaggattg

SEQ ID NO: 41 - DNA, 14 bp, artificial sequence
  Site specific nuclease binding site (SBS18473)
tgggcgggagtctt

SEQ ID NO: 42 - DNA, 14 bp, artificial sequence
  Site specific nuclease binding site (SBS18473)
aagactcccgccca

SEQ ID NO: 43 - DNA, 26 bp, artificial sequence
  primer sequence
acaagagtggattgatgatctagaga

SEQ ID NO: 44 - DNA, 26 bp, artificial sequence
  primer sequence
ctttgatgcctatgtgacacgtaaac

SEQ ID NO: 45 - DNA, 29 bp, artificial sequence
  primer sequence
ccagcgtaagcaataccagccacaacacc

SEQ ID NO: 46 - DNA, 18 bp, artificial sequence
  primer sequence
ttcagcacccgtcagaat

SEQ ID NO: 47 - DNA, 17 bp, artificial sequence
  primer sequence
tggtcgccatagcttgt

SEQ ID NO: 48 - DNA, 20 bp, artificial sequence
  primer sequence
tgccgagaacttgaggaggt

SEQ ID NO: 49 - DNA, 22 bp, artificial sequence
  primer sequence
tggttatgacaggctccgttta

SEQ ID NO: 50 - DNA, 23 bp, artificial sequence
  primer sequence
aacaaacctcctggctacttcaa

SEQ ID NO: 51 - DNA, 17 bp, artificial sequence
  primer sequence
cttgctggtgttatgtg

SEQ ID NO: 52 - DNA, 20 bp, artificial sequence
  primer sequence
tgttcggttccctctaccaa

SEQ ID NO: 53 - DNA, 22 bp, artificial sequence
  primer sequence
caacatccatcaccttgactga

SEQ ID NO: 54 - DNA, 24 bp, artificial sequence
  primer sequence
cacagaaccgtcgcttcagcaaca

SEQ ID NO: 55 - DNA, 20 bp, artificial sequence
  primer sequence
gtcgaggaactgctcattgg

SEQ ID NO: 56 - DNA, 21 bp, artificial sequence
  primer sequence
cagaagttgatctcgccgtta

SEQ ID NO: 57 - DNA, 21 bp, artificial sequence
  primer sequence
cgtgttgggaaagaacttgga

SEQ ID NO: 58 - DNA, 18 bp, artificial sequence
  primer sequence
ccgtggttggcttggtct

SEQ ID NO: 59 - DNA, 15 bp, artificial sequence
  primer sequence
cactccccactgcct

SEQ ID NO: 60 - DNA, 21 bp, artificial sequence
  primer sequence
cgccgaagtatcgactcaact

SEQ ID NO: 61 - DNA, 19 bp, artificial sequence
  primer sequence
gcaacgtcggttcgagatg

SEQ ID NO: 62 - DNA, 24 bp, artificial sequence
  primer sequence
tcagaggtagttggcgtcatcgag

SEQ ID NO: 63 - DNA, 23 bp, artificial sequence
  primer sequence
ataacgtgccttggagtatttgg

SEQ ID NO: 64 - DNA, 22 bp, artificial sequence
  primer sequence
tggagtgaagcagatgatttgc

SEQ ID NO: 65 - DNA, 18 bp, artificial sequence
  primer sequence
ttgcatccatcttgttgc

SEQ ID NO: 66 - DNA, 18 bp, artificial sequence
  primer sequence
tggcggacgacgacttgt

SEQ ID NO: 67 - DNA, 19 bp, artificial sequence
  primer sequence
aaagtttggaggctgccgt

SEQ ID NO: 68 - DNA, 21 bp, artificial sequence
  primer sequence
cgagcagaccgccgtgtactt

SEQ ID NO: 69 - DNA, 18 bp, artificial sequence
  primer sequence
aggaggcaccacgaaaac

SEQ ID NO: 70 - DNA, 20 bp, artificial sequence
  primer sequence
gtcaaagagaggcggcatga

SEQ ID NO: 71 - DNA, 26 bp, artificial sequence
  primer sequence
gatttctgcatcacaggttccttttg

SEQ ID NO: 72 - DNA, 18 bp, artificial sequence
  primer sequence
aagtcgatcacggctaga

SEQ ID NO: 73 - DNA, 18 bp, artificial sequence
  primer sequence
aagtcgatcacggctaga

SEQ ID NO: 74 - DNA, 23 bp, artificial sequence
  primer sequence
aacaaacctcctggctacttcaa

(19) United States
(12) Patent Application Publication
(10) Pub. No.: US 2018/0142249 A1
(43) Pub. Date: May 24, 2018

(54) SITE SPECIFIC INTEGRATION OF A TRANSGNE USING INTRA-GENOMIC RECOMBINATION VIA A NON-HOMOLOGOUS END JOINING REPAIR PATHWAY

(71) Applicant: Dow AgroSciences LLC, Indianapolis, IN (US)

(72) Inventors: Sandeep Kumar, Carmel, IN (US); Joseph F. Petolino, Zionsville, IN (US); Andrew F. Worden, Indianapolis, IN (US); Pierluigi Barone, Zionsville, IN (US); Matthew A. Simpson, Brownsburg, IN (US); Tonya L. Strange, Brownsburg, IN (US)

(73) Assignee: Dow AgroSciences LLC, Indianapolis, IN (US)

(21) Appl. No.: 15/797,285

(22) Filed: Oct. 30, 2017

Related U.S. Application Data
(60) Provisional application No. 62/424,574, filed on Nov. 21, 2016.

(51) Int. Cl. C12N 15/82

(52) U.S. Cl. CPC C12N 15/8213; C12N 15/8222; C12N 15/8234; C12N 15/8209; C12N 15/8261; C12N 15/8286; C12N 15/8274; C12N 15/8231

(57) ABSTRACT

Compositions and methods to modify at least one target locus in a plant cell are provided, which comprises providing a plant cell, a plant, or a plant part with one or more target loci and one or more donor loci, providing at least one cleaving site specific nuclease to produce a double strand break within the target loci, followed by non-homologous end joining of at least one donor locus within at least one target locus. Target loci, donor loci and nuclease loci used in these methods, and plant cells, plants and plant parts comprising these target loci, donor loci, nuclease loci and/or the recombined loci are also provided.

### CROSS REFERENCE TO RELATED APPLICATION

The present application claims priority to the benefit of U.S. Provisional Patent Application Ser. No. 62/424,574 filed Nov. 21, 2016 the disclosure of which is hereby incorporated by reference in its entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: one 88.3 KB ASCII (Text) file named “76767 FINAL SEQ_ST25” created on Oct. 12, 2017.

### BACKGROUND

Precise, robust, and reproducible techniques for site-directed integration of transgenes into plant genomes have been a longtime goal in developing transgenic plants. Traditional transformation methodologies rely upon the random introduction of transgenes within a plant genome. Unfortunately, these methodologies can be limited in application, especially since the majority of elite crop varieties are poorly transformable. The culmination of such technical hurdles results in inefficient transformation of a transgene within undesirable locations of the plant genome. Site specific integration of transgenes within plants through the use of site specific nucleases has recently developed as a promising solution for integrating a transgene within a specific genomic location. However, this technology is still somewhat limited by low transformation efficiency. Therefore, a need exists for development of plant transformation technologies that allow for site specific integration of transgenes with robust efficiency.

### BRIEF DESCRIPTION OF THE INVENTION

In an embodiment, the present disclosure is directed to a method for inserting an integrated donor DNA within a plant genomic target locus by providing a first viable plant containing a genomic DNA, the genomic DNA comprising the donor DNA flanked by a plurality of recognition sequences and the plant genomic target locus, wherein the plant genomic target locus comprises at least one recognition sequence; providing a second viable plant containing a genomic DNA, the genomic DNA comprising a DNA encoding at least one zinc finger nuclease engineered to cleave the genomic DNA at the recognition sequence; crossing the first and second viable plants such that F1 seed is produced on either the first or the second viable plant; expressing the zinc finger nuclease within the F1 seed or a F1 plant, wherein the expressed zinc finger nuclease cleaves the donor DNA and the genomic DNA at the recognition sequence; and, growing the resultant F1 plant containing a genomic DNA, wherein the donor DNA is integrated within the recognition sequence of the plant genomic target locus via non-homologous end joining. In an aspect of this embodiment, the recognition sequence comprises at least one recognition sequence. In further aspect, the recognition sequence comprises first and second recognition sequences. In other aspects, the first and second recognition sequences are identical. In subsequent aspects, the zinc finger nuclease is provided by crossing the first and second viable plants such that the zinc finger nuclease cleaves both recognition sequences. In other aspects, the donor DNA and the plant genomic target locus are unlinked. In additional aspects, the donor DNA and the plant genomic target locus are located on homologous chromosomes. In further aspects, the donor DNA and the plant genomic target locus are located on non-homologous chromosomes. In an embodiment, the plant genomic target locus comprises an expression cassette. In aspects of this embodiment, the expression cassette is located between the first and second recognition sequences. In another aspect of this embodiment, the expression cassette is located outside of the first recognition sequence. In a further aspect of this embodiment, the expression cassette is located outside of the second recognition sequence. In another embodiment, the first viable plant is homozygous for at least one genomic target locus. In an additional embodiment, the first viable plant is homozygous for at least one donor DNA. In an embodiment, the first viable plant is heterozygous for at least one genomic target locus. In an embodiment, the first viable plant is heterozygous for at least one donor DNA. In further embodiments, the plant genomic target locus is a transgenic locus. In other embodiments, the plant genomic target locus is an endogenous locus. In some aspects, the zinc finger nuclease is driven by a promoter. Exemplary promoters include a pollen-specific promoter, a seed-specific promoter, and/or a developmental-stage specific promoter. In a further embodiment, the donor DNA comprises a selectable marker.

In an embodiment, the present disclosure is directed to a method for transmitting a transgene into other plants by: crossing a first plant regenerated from a plant cell or tissue transformed with an isolated nucleic acid molecule comprising a genomic target locus and the transgene with a second plant regenerated from a plant cell or tissue transformed with an isolated nucleic acid molecule comprising a promoter operably linked to a zinc finger nuclease; expressing the zinc finger nuclease so that a first zinc finger nuclease monomer is paired with a second zinc finger nuclease monomer; obtaining a F1 plant resulting from the cross wherein the transgene is specifically and stably integrated within the genomic target locus via non-homologous end joining; and, cultivating the F1 plant resulting from the cross. In an aspect of this embodiment, the plant regenerated from the plant cell or tissue transformed with the isolated nucleic acid molecule comprising the promoter operably linked to the zinc finger nuclease comprises at least one zinc finger nuclease monomer. In another aspect, the plant regenerated from the plant cell or tissue transformed with the isolated nucleic acid molecule comprising the promoter operably linked to the zinc finger nuclease comprises the first and the second zinc finger nuclease monomers. In subsequent aspects, the plant regenerated from the plant cell or tissue transformed with the isolated nucleic acid molecule comprising the promoter operably linked to the zinc finger nuclease comprises the first zinc finger nuclease monomer. In other aspects, the plant regenerated from the plant cell or tissue transformed with the isolated nucleic acid molecule comprising the genomic target locus and the transgene further comprises an isolated nucleic acid molecule comprising a promoter operably linked to a second zinc finger nuclease, wherein the second zinc finger nuclease comprises the second zinc finger nuclease monomer. In another aspect, the first and second zinc finger nuclease monomers of result in the release of the transgene and cleavage of the genomic target locus through double strand breaks.

In an embodiment, the present disclosure is directed to an F1 plant that is produced using a method of the disclosure. In an aspect of this embodiment, the F1 plant comprises a transgenic event. In an embodiment, the transgenic event is an insecticidal resistance trait, herbicide tolerance trait, nitrogen use efficiency trait, water use efficiency trait, nutritional quality trait, DNA binding trait, small RNA trait, selectable marker trait, or any combination thereof. In some embodiments the transgenic event is an agronomic trait. In some embodiments, the transgenic event is a herbicide tolerant trait. A non-limiting example of a herbicide tolerant trait is a dgt-28 trait, an aad-1 trait, or an aad-12 trait. In other aspects of this embodiment, the transgenic plant produces a commodity product. In an embodiment, the commodity product can include protein concentrate, protein isolate, grain, meal, flour, oil, and/or fiber as non-limiting examples of commodity products. In an additional aspect of this embodiment, the transgenic plant is a monocotyledonous plant. A non-limiting example of a monocotyledonous plant is a Zea mays plant. In an additional aspect of this embodiment, the transgenic plant is a dicotyledonous plant. A non-limiting example of a dicotyledonous plant is a tobacco plant.

In an embodiment, the present disclosure is directed to a method for inserting a donor DNA within a plant genomic target locus by: acquiring a viable plant cell containing the plant genomic target locus, wherein the plant genomic target locus comprises a recognition sequence; providing a donor DNA, the donor DNA comprising at least one recognition sequence flanking the donor DNA; providing and expressing a site specific nuclease, wherein the expressed site specific nuclease cleaves the plant genomic target locus and the donor DNA at the recognition sequence; and obtaining a resultant plant cell, wherein the donor DNA is integrated within the recognition sequence of the plant genomic target locus via non-homologous end joining. In an aspect of this method, the donor DNA is integrated within the recognition sequence of the plant genomic target locus via non-homologous end joining during a phase of the cell cycle. In an aspect of this method, the phase of the cell cycle is selected from the group consisting of the gap 2 (G2) cell cycle phase, the gap 1 (G1) cell cycle phase, the DNA synthesis (S phase) cell cycle phase, the mitosis (M) cell cycle phase, and any combination thereof. In a further aspect of this method, the site specific nuclease is selected from the group consisting of a zinc finger nuclease, a CRISPR, a TALEN, a meganuclease, a CRE recombinase, and any combination thereof. In a further aspect of this method, the site specific nuclease is selected from the group consisting of a zinc finger nuclease, a CRISPR, a TALEN, a meganuclease, a CRE recombinase, and any combination thereof.

In an embodiment, the present disclosure is directed to a method for intra genomic recombination mobilization of a donor DNA fragment from a parental plant into the target locus of an F1 progeny plant. In an aspect of this method, the donor DNA is integrated within the target locus via one sided invasion (OSI) of the donor DNA fragment within the target locus. The target locus may be a genomic locus, a mitochondrial genomic locus or a chloroplast genomic locus. In further aspects, the insertion of the donor DNA may be facilitated by double strand breaks produced from a site specific nuclease. Non-limiting examples of such a site specific nuclease include; CRISPR cas9, CRISPR cpf1, TALENS, and zinc finger nucleases. In some aspects, the double stranded breaks may occur on either side of the donor DNA. In other aspects, the double stranded breaks may occur at the target locus. In an additional aspect, the donor DNA may integrate within the target locus during a phase of the cell cycle. Exemplary phases of the cell cycle may include the gap 2 (G2) cell cycle phase, the gap 1 (G1) cell cycle phase, the DNA synthesis (S phase) cell cycle phase, the mitosis (M) cell cycle phase, and any combination thereof. In some aspects, the method includes a parental plant that comprises the donor DNA fragment. In other aspects, the method includes a parental plant that comprises the site specific nuclease. Accordingly, a first parental plant comprising the donor DNA may be crossed with a second parental plant comprising the site specific nuclease. The result of such a cross produces an F1 progeny plant. In some aspects, the F1 progeny plant comprises the donor DNA that is integrated within the target locus via OSI mediated insertion.

In an embodiment, the present disclosure is directed to a method for NHEJ-mediated integration of a donor DNA within a plant genomic target locus, by: providing a first viable plant containing a genomic DNA, the DNA comprising the donor DNA flanked by a plurality of recognition sequences and the plant genomic target locus, wherein the plant genomic target locus comprises at least one recognition sequence; providing a second viable plant containing a genomic DNA, the DNA comprising a transgene encoding a site specific nuclease designed to cleave the recognition sequence; crossing the first and second viable plants to produce an F1 progeny; generating an F1 progeny, wherein the F1 progeny seed is grown to maturity; expressing the site specific nuclease within the F1 progeny during a phase of the cell cycle; cleaving the donor DNA and the plant genomic target locus with the site specific nuclease; integrating the donor DNA within the plant genomic target locus via a NHEJ-mediated integration mechanism, wherein the integration of the donor DNA within the plant genomic target locus occurs during the phase of the cell cycle; and obtaining an F1 plant with the donor DNA integrated within the plant genomic target locus. In an aspect of this method, the phase of the cell cycle is selected from the group consisting of the gap 2 (G2) cell cycle phase, the gap 1 (G1) cell cycle phase, the DNA synthesis (S phase) cell cycle phase, the mitosis (M) cell cycle phase, and any combination thereof. In a further aspect of this method, the site specific nuclease is selected from the group consisting of a zinc finger nuclease, a CRISPR, a TALEN, a meganuclease, a CRE recombinase, and any combination thereof.

In an embodiment, the present disclosure is directed to a method for inserting a donor DNA within a target locus of a plant genome, by: providing at least one donor DNA flanked by a plurality of recognition sequences stably integrated within the plant genome, wherein the recognition sequences of the donor DNA are also present within the target locus; providing at least one zinc finger nuclease engineered to cleave the genomic DNA at the recognition sequence stably integrated within the plant genome; expressing the zinc finger nuclease, wherein the expressed zinc finger nuclease cleaves the donor DNA and the target locus at the recognition sequence; and, obtaining the resultant plant genome, wherein the donor DNA is integrated within the recognition sequence of the target locus via non-homologous end joining. In an aspect of this method, the donor DNA is stably integrated within the plant genome by a first plant transformation method. In an aspect of this method, the zinc finger nuclease is stably integrated within the plant genome by a second plant transformation method. In an aspect of this method, an additional step of cultivating a whole plant comprising the donor DNA is included. In an aspect of this method, an additional step of cultivating a whole plant comprising the zinc finger nuclease is included.

In addition to the exemplary aspects and embodiments described above, further aspects and embodiments will become apparent by study of the following descriptions.

### BRIEF DESCRIPTION OF THE FIGURES AND SEQUENCE LISTING

The nucleic acid sequences listed in the accompanying sequence listing are shown using standard letter abbreviations for nucleotide bases, as defined in 37 C.F.R. § 1.822. Only one strand of each nucleic acid sequence is shown, but the complementary strand is understood to be included by any reference to the displayed strand in the accompanying sequence listing.

FIG. 1 depicts a plasmid map of pDAB1585.

FIG. 2 depicts a plasmid map of pDAB118259.

FIG. 3 depicts a plasmid map of pDAB118257.

FIG. 4 depicts a plasmid map of pDAB118261.

FIG. 5 depicts a schematic of the process used for crossing two parental plants according to the subject disclosure.

FIG. 6 depicts the resulting introgression of the donor (i.e., labeled as “NHEJ Donor” and “HDR Donor”) within a target genomic locus (i.e., labeled as “Target”) and the resulting integrant (i.e., labeled as “Targeted”). Further provided in FIG. 6 is a gel electrophoresis of the resulting integrations as indicated by PCR amplicons.

FIG. 7 depicts a plasmid map of pDAB118253.

FIG. 8 depicts a plasmid map of pDAB118254.

FIG. 9 depicts a plasmid map of pDAB113068.

FIG. 10 depicts a plasmid map of pDAB105825.

FIG. 11 depicts a plasmid map of pDAB118280.

FIG. 12 depicts a schematic of the intragenomic recombination process via homology directed repair.

FIG. 13 depicts a schematic of the intragenomic recombination process via non homologous end joining repair.

FIG. 14 depicts a schematic of the intragenomic recombination process via one sided invasion (OSI).

FIG. 15 depicts a schematic of the in planta directed recombination that results from crossing a first viable parental plant with a second viable parental plant to produce progeny (F1) plants via an intra genomic recombination.

FIG. 16 depicts the resulting introgression of the donor (i.e., labeled as “NHEJ Donor Plant” and “HDR Donor Plant”) within a target genomic locus (i.e., labeled as “Target Plant”) and the resulting integrant (i.e., labeled as “Targeted Plant”). Further provided in FIG. 16 is a gel electrophoresis of the resulting integrations as indicated by PCR amplicons.

FIG. 17 depicts the resulting introgression of the donor (i.e., labeled as “OSI Donor Plant”) within a target genomic locus (i.e., labeled as “Target Plant”) and the resulting integrant (i.e., labeled as “Targeted Plant”). Gel electrophoresis of the resulting integrations as indicated by PCR amplicons.

### DETAILED DESCRIPTION OF THE INVENTION

Overview:

Disclosed herein are methods and compositions for integrating donor polynucleotide sequences within a plant genome. In certain embodiments, the subject disclosure relates to a breeding strategy for in planta mobilization of a donor polynucleotide within a specific locus of the plant genome. In some aspects of this embodiment, the donor polynucleotide sequence is integrated within the plant genome via a Non-Homologous End Joining (NHEJ) mediated cellular mechanism. In some aspects of this embodiment, the donor polynucleotide sequence is integrated within the plant genome via a Non-Homologous End Joining (NHEJ) mediated cellular mechanism on one side of the donor sequence and a Homology Directed Repair (HDR) mediated cellular mechanism on the other side of the donor sequence. In further aspects of this embodiment, the donor polynucleotide is targeted within a specific genomic locus following the crossing of two parent plants. Further aspects of this embodiment involves the targeted genome rearrangement following: i) concurrent double strand break formation at donor and target loci, ii) donor template sequence excision, and iii) non-homology directed repair at the target locus. Ultimately, the randomly integrated donor sequence becomes integrated into the target locus. The development of novel targeting methods allows for the rapid development of parental lines containing polynucleotide donor sequences, site specific nuclease binding sequences, and site specific nucleases through conventional plant transformation technologies. These parental lines can be utilized for the in planta targeted delivery of donor within a specific locus of the plant genome and site specific nucleases to circumvent technical problems associated with inefficient transformation methods and the low frequency of site-specific versus random DNA integration. Furthermore, the in planta targeting delivery of donor and site specific nuclease allows the concurrent cleavage and integration of the target and donor within the progeny plants occurs at all various cell cycle stages (G1, S, G2, and M), thereby resulting in donor mobilization into the genomic target locus via the DNA repair and recombination machinery that is functional at such cell cycle stages.

The in planta targeting via non-homologous end joining (NHEJ) repair would represent an improved means of site-specific DNA integration and transgene stacking. Upon delivery of the sites specific nuclease, the genomic locus and flanking sequences from the donor can be cleaved by double strand breaks. The resulting donor sequence is thereby excised and is available for integration within the cleaved genomic locus. Upon NHEJ repair of the target genomic locus using the excised donor template, the donor would be specifically integrated within a site specific locus. The subject disclosure provides methods and compositions for precisely integrating a genomic donor sequence within a genomic locus via an NHEJ mediated cellular mechanism.

### Definitions

The definitions and methods provided define the present invention and guide those of ordinary skill in the art in the practice of the present invention. Unless otherwise noted, terms are to be understood according to conventional usage by those of ordinary skill in the relevant art. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference, unless only specific sections of patents or patent publications are indicated to be incorporated by reference.

In order to further clarify this disclosure, the following terms, abbreviations and definitions are provided.

The term “about” is used herein to mean approximately, roughly, around, or in the region of. When the term “about” is used in conjunction with a numerical range, it modifies that range by extending the boundaries above and below the numerical values-set forth. In general, the term “about” is used herein to modify a numerical value above and below the stated value by a variance of 20 percent up or down (higher or lower), preferably 15 percent, more preferably 10 percent and most preferably 5 percent.

As used herein, the terms “comprises”, “comprising”, “includes”, “including”, “has”, “having”, “contains”, or “containing”, or any other variation thereof, are intended to be non-exclusive or open-ended. For example, a composition, a mixture, a process, a method, an article, or an apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term “invention” or “present invention” as used herein is a non-limiting term and is not intended to refer to any single embodiment of the particular invention but encompasses all possible embodiments as disclosed in the application.

The term “genome” or “genomic DNA” as used herein refers to the heritable genetic information of a host organism. Said genomic DNA comprises the entire genetic material of a cell or an organism, including the DNA of the nucleus (chromosomal DNA), extrachromosomal DNA, and organellar DNA (e.g. of mitochondria and plastids like chloroplasts). Preferably, the terms genome or genomic DNA is referring to the chromosomal DNA of the nucleus.

The term “chromosomal DNA” or “chromosomal DNA sequence” as used herein is referring to the genomic DNA of the cellular nucleus independent from the cell cycle status. Chromosomal DNA might therefore be organized in chromosomes or chromatids that might be either condensed or uncoiled.

As used herein the terms “native” or “natural” define a condition found in nature. A “native DNA sequence” is a DNA sequence present in nature that was produced by natural means or traditional breeding techniques but not generated by genetic engineering (e.g., using molecular biology/transformation techniques).

As used herein, “endogenous” as it relates to nucleic acid or amino acid sequences refers to the native form of a polynucleotide, gene or polypeptide in its natural location in the organism or in the genome of an organism. An “endogenous” molecule is one that is normally present in a particular cell at a particular developmental stage under particular environmental conditions. For example, an endogenous, nucleic acid can comprise a chromosome, the genome of a mitochondrion, chloroplast or other organelle, or a naturally-occurring episomal nucleic acid. Additional endogenous molecules can include proteins, for example, transcription factors and enzymes.

As used herein an “exogenous sequence” refers to a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a coding sequence for any polypeptide or fragment thereof, a functioning version of a malfunctioning endogenous molecule or a malfunctioning version of a normally-functioning endogenous molecule. Additionally, an exogenous molecule can comprise a coding sequence from another species that is an ortholog of an endogenous gene in the host cell.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, site specific nuclease protein, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced, into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, nanoparticle transformation, DEAE-dextran-mediated transfer and viral vector-mediated transfer.

The term “chimeric” as used herein, refers to a sequence that is comprised of sequences that are “recombined”. For example the sequences are recombined and are not found together in nature.

The term “recombine” or “recombination” as used herein means refers to any method of joining polynucleotides. The term includes end to end joining, and insertion of one sequence into another. The term is intended to encompass includes physical joining techniques such as sticky-end ligation and blunt-end ligation. Such sequences may also be artificially or recombinantly synthesized to contain the recombined sequences. Additionally, the term can encompass the integration of one sequence within a second sequence, for example the integration of a polynucleotide within the genome of an organism by homologous recombination can result from “recombination”. For the purposes of the subject disclosure, the term “homologous recombination” is used to indicate recombination occurring as a consequence of interaction between segments of genetic material that are homologous. In contrast, for purposes of the subject disclosure, the term “non-homologous recombination” is used to indicate a recombination occurring as a consequence of interaction between segments of genetic material that are not homologous, or identical. Non-homologous end joining (NHEJ) is an example of non-homologous recombination. In further aspects the term refers to the reassortment of sections of DNA or RNA sequences between two DNA or RNA molecules. “Homologous recombination” occurs between two DNA molecules which hybridize by virtue of homologous or complementary nucleotide sequences present in each DNA molecule.

As used herein, the term “homologous region” is not limited to a given single polynucleotide sequence, but may comprise parts of, or complete sequences of promoters, coding regions, terminator sequences, enhancer sequences, matrix-attachment regions, or one or more expression cassettes. The term “homologous region” gains meaning in combination with another “homologous region” by sharing sufficient sequence identity to be able to recombine via homologous recombination with such other homologous region. Because a homologous region is not limited by any structural features other than its sufficient sequence identity to another homologous region, it may be that a given sequence may be a homologous region A to a homologous region B, but may at the same time be a homologous region X to a homologous region Y. Thus, a homologous region of a donor locus has to be understood in context to another homologous region of a target locus or another sequence of the same donor locus, for example a given sequence may be a homologous region A of a donor locus if used in combination with a target locus comprising a homologous region B.

The term “isolated”, as used herein means having been removed from its natural environment.

The term “purified”, as used herein relates to the isolation of a molecule or compound in a form that is substantially free of contaminants normally associated with the molecule or compound in a native or natural environment and means having been increased in purity as a result of being separated from other components of the original composition. The term “purified nucleic acid” is used herein to describe a nucleic acid sequence which has been separated from other compounds including, but not limited to polypeptides, lipids and carbohydrates.

As used herein, the terms “polynucleotide”, “nucleic acid”, and “nucleic acid molecule” are used interchangeably, and may encompass a singular nucleic acid; plural nucleic acids; a nucleic acid fragment, variant, or derivative thereof; and nucleic acid construct (e.g., messenger RNA (mRNA) and plasmid DNA (pDNA)). A polynucleotide or nucleic acid may contain the nucleotide sequence of a full-length cDNA sequence, or a fragment thereof, including untranslated 5′ and/or 3′ sequences and coding sequence(s). A polynucleotide or nucleic acid may be comprised of any polyribonucleotide or polydeoxyribonucleotide, which may include unmodified ribonucleotides or deoxyribonucleotides or modified ribonucleotides or deoxyribonucleotides. For example, a polynucleotide or nucleic acid may be comprised of single- and double-stranded DNA; DNA that is a mixture of single- and double-stranded regions; single- and double-stranded RNA; and RNA that is mixture of single- and double-stranded regions. Hybrid molecules comprising DNA and RNA may be single-stranded, double-stranded, or a mixture of single- and double-stranded regions. The foregoing terms also include chemically, enzymatically, and metabolically modified forms of a polynucleotide or nucleic acid.

It is understood that a specific DNA or polynucleotide refers also to the complement thereof, the sequence of which is determined according to the rules of deoxyribonucleotide base-pairing. Although only one strand of DNA may be presented in the sequence listings of this disclosure, those having ordinary skill in the art will recognize that the complementary strand can be ascertained and determined from the strand presented herein. Accordingly, a single strand of a polynucleotide can be used to determine the complementary strand, and, accordingly, both strands (i.e., the sense strand and anti-sense strand) are exemplified from a single strand.

As used herein, the term “gene” refers to a nucleic acid that encodes a functional product (RNA or polypeptide/protein). A gene may include regulatory sequences preceding (5′ non-coding sequences) and/or following (3′ non-coding sequences) the sequence encoding the functional product.

“Transgene”, “transgenic” or “recombinant” as used herein refers to a polynucleotide manipulated by man or a copy or complement of a polynucleotide manipulated by man. For instance, a transgenic expression cassette comprising a promoter operably linked to a second polynucleotide may include a promoter that is heterologous to the second polynucleotide as the result of manipulation by man (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)) of an isolated nucleic acid comprising the expression cassette. In another example, a recombinant expression cassette may comprise polynucleotides combined in such a way that the polynucleotides are extremely unlikely to be found in nature. For instance, restriction sites or plasmid vector sequences manipulated by man may flank or separate the promoter from the second polynucleotide. One of skill will recognize that polynucleotides can be manipulated in many ways and are not limited to the examples below. In one example, a transgene is a gene sequence (e.g., a herbicide-resistance gene), a gene encoding an industrially or pharmaceutically useful compound, or a gene encoding a desirable agricultural trait. In yet another example, the transgene is an antisense nucleic acid sequence, wherein expression of the antisense nucleic acid sequence inhibits expression of a target nucleic acid sequence. A transgene may contain regulatory sequences operably linked to the transgene (e.g., a promoter).

As used herein, the term “coding sequence” refers to a nucleic acid sequence that encodes a specific amino acid sequence. A “regulatory sequence” refers to a nucleotide sequence located upstream (e.g., 5′ non-coding sequences), within, or downstream (e.g., 3′ non-coding sequences) of a coding sequence, which influence the transcription, RNA processing or stability, or translation of the coding sequence. Regulatory sequences include, for example and without limitation associated: promoters; translation leader sequences; introns; polyadenylation recognition sequences; RNA processing sites; effector binding sites; and stem-loop structures.

As used herein, the term “polypeptide” includes a singular polypeptide, plural polypeptides, and fragments thereof. This term refers to a molecule comprised of monomers (amino acids) linearly linked by amide bonds (also known as peptide bonds). The term “polypeptide” refers to any chain or chains of two or more amino acids, and does not refer to a specific length or size of the product. Accordingly, peptides, dipeptides, tripeptides, oligopeptides, protein, amino acid chain, and any other term used to refer to a chain or chains of two or more amino acids, are included within the definition of “polypeptide”, and the foregoing terms are used interchangeably with “polypeptide” herein. A polypeptide may be isolated from a natural biological source or produced by recombinant technology, but a specific polypeptide is not necessarily translated from a specific nucleic acid. A polypeptide may be generated in any appropriate manner, including for example and without limitation, by chemical synthesis. Likewise, a polypeptide may be generated by expressing a native coding sequence, or portion thereof, that are introduced into an organism in a form that is different from the corresponding native coding sequence.

As used herein the term “heterologous” refers to a polynucleotide, gene or polypeptide that is not normally found at its location in the reference (host) organism. For example, a heterologous nucleic acid may be a nucleic acid that is normally found in the reference organism at a different genomic location. By way of further example, a heterologous nucleic acid may be a nucleic acid that is not normally found in the reference organism. A host organism comprising a heterologous polynucleotide, gene or polypeptide may be produced by introducing the heterologous polynucleotide, gene or polypeptide into the host organism. In particular examples, a heterologous polynucleotide comprises a native coding sequence, or portion thereof, that is reintroduced into a source organism in a form that is different from the corresponding native polynucleotide. In particular examples, a heterologous gene comprises a native coding sequence, or portion thereof, that is reintroduced into a source organism in a form that is different from the corresponding native gene. For example, a heterologous gene may include a native coding sequence that is a portion of a chimeric gene including non-native regulatory regions that is reintroduced into the native host. In particular examples, a heterologous polypeptide is a native polypeptide that is reintroduced into a source organism in a form that is different from the corresponding native polypeptide.

A heterologous gene or polypeptide may be a gene or polypeptide that comprises a functional polypeptide or nucleic acid sequence encoding a functional polypeptide that is fused to another gene or polypeptide to produce a chimeric or fusion polypeptide, or a gene encoding the same. Genes and proteins of particular embodiments include specifically exemplified full-length sequences and portions, segments, fragments (including contiguous fragments and internal and/or terminal deletions compared to the full-length molecules), variants, mutants, chimerics, and fusions of these sequences.

As used herein the term “nucleic acid molecule” refers to a polymeric form of nucleotides, which can include both sense and anti-sense strands of RNA, cDNA, genomic DNA, and synthetic forms and mixed polymers of the above. A nucleotide refers to a ribonucleotide, deoxynucleotide, or a modified form of either type of nucleotide. A “nucleic acid molecule” as used herein is synonymous with “nucleic acid” and “polynucleotide.” The term includes single- and double-stranded forms of DNA. A nucleic acid molecule can include either or both naturally occurring and modified nucleotides linked together by naturally occurring and/or non-naturally occurring nucleotide linkages.

Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications, such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., peptides), intercalators (e.g., acridine, psoralen, etc.), chelators, alkylators, and modified linkages (e.g., alpha anomeric nucleic acids, etc.). The term “nucleic acid molecule” also includes any topological conformation, including single-stranded, double-stranded, partially duplexed, triplexed, hairpinned, circular, and padlocked conformations.

The term “sequence” refers to any series of nucleic acid bases or amino acid residues, and may or may not refer to a sequence that encodes or denotes a gene or a protein. Many of the genetic constructs used herein are described in terms of the relative positions of the various genetic elements to each other.

As used herein, the term “plant” includes a whole plant and any descendant, cell, tissue, or part of a plant. The term “plant parts” include any part(s) of a plant, including, for example and without limitation: seed (including mature seed, immature seed, and immature embryo without testa); a plant protoplast; a plant cutting; a plant cell; a plant cell culture; a plant organ (e.g., including, but not limited to, stems, roots, shoots, fruits, ovules, stamens, leaves, embryos, meristematic regions, callus tissue, gametophytes, sporophytes, pollen, embryos, microspores, hypocotyls, cotyledons, flowers, fruits, anthers, sepals, petals, pollen, seeds, related explants and the like). A plant tissue or plant organ may be a seed, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root.

A plant cell is the structural and physiological unit of the plant. Plant cells, as used herein, includes protoplasts and protoplasts with a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant part” in embodiments herein.

The term “promoter” as used herein refers to regions or sequences located upstream and/or down-stream from the start of transcription and which are involved in recognition and binding of RNA polymerase and other proteins to initiate transcription. Promoters permit the proper activation or repression of the gene which they control. A promoter contains specific sequences that are recognized by transcription factors. These factors bind to the promoter DNA sequences and result in the recruitment of RNA polymerase, the enzyme that synthesizes the RNA from the coding region of the gene. A “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is only active in specific types of tissues or cells, while a “tissue preferred” promoter is preferentially, but not exclusively, active in certain tissues or cells. A “promoter which is active in plants or plant cells” is a promoter which has the capability of initiating transcription in plant cells. In some embodiments, tissue-specific promoters are used in methods of the invention, e.g., a pollen-specific promoter.

The term “close to” or “proximal” when used in reference to the location of one element of a target locus or a donor locus in respect to another element of a target locus or a donor locus, e.g. a rare cleaving nuclease cutting site, a homologous region, a region Z or an expression cassette for a marker gene or rare cleaving nuclease or any other element of a target locus or donor locus, means a distance of not more than 50 bp, 100 bp, 200 bp, 300 bp, 400 bp, 500 bp, 600 bp, 700 bp, 800 bp, 900 bp, 1000 bp, 2000 bp, 3000 bp, 4000 bp, 5000 bp, 6000 bp 7000 bp, 8000 bp, 9000 bp, or not more than 10000 bp.

The term “expression cassette” or “gene expression cassette”—for example when referring to the expression cassette for the site specific nuclease—means those constructions in which the DNA to be expressed is linked operably to at least one genetic control element which enables or regulates its expression (i.e. transcription and/or translation). Here, expression may be for example stable or transient, constitutive or inducible. Furthermore, the term refers to a promoter operably linked to a gene (e.g., a transgene), that is further operably linked to a 3′-UTR termination sequence. Multiple gene expression cassettes may be stacked with one another.

The term “operably linked” refers the relation of a first nucleotide sequence with a second nucleotide sequence when the first nucleotide sequence is in a functional relationship with the second nucleotide sequence. For instance, a promoter is operably linked to a coding sequence if the promoter affects the transcription or expression of the coding sequence. When recombinantly produced, operably linked nucleotide sequences are generally contiguous and, where necessary to join two protein-coding regions, in the same reading frame. However, nucleotide sequences need not be contiguous to be operably linked.

The term, “operably linked,” when used in reference to a regulatory sequence and a coding sequence, means that the regulatory sequence affects the expression of the linked coding sequence. “Regulatory sequences,” “regulatory elements”, or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

When used in reference to two or more amino acid sequences, the term “operably linked” means that the first amino acid sequence is in a functional relationship with at least one of the additional amino acid sequences.

The term “integrated DNA” or “integrated donor DNA” refers to a DNA that is inserted within a genome. In most embodiment the incorporation of this DNA within the genome occurs such that the integrated DNA can be transmitted to progeny through normal cellular reproduction. The term is often used to confirm that successful targeting of foreign or exogenous DNA into the target locus of an organism's genome.

The term “expression” and “gene expression” are used interchangeably and refer to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “transform” or “transduce” refers to the process of transferring nucleic acid molecules into the cell. A cell is “transformed” by a nucleic acid molecule transduced into the cell when the nucleic acid molecule becomes stably replicated by the cell, either by incorporation of the nucleic acid molecule into the cellular genome, or by episomal replication. As used herein, the term “transformation” encompasses all techniques by which a nucleic acid molecule can be introduced into such a cell. Examples include, but are not limited to, transfection with viral vectors, transformation with plasmid vectors, electroporation (Fromm et al. (1986) Nature 319:791-3), lipofection (Feigner et al. (1987) Proc. Natl. Acad. Sci. USA 84:7413-7), microinjection (Mueller et al. (1978) Cell 15:579-85), Agrobacterium-mediated transfer (Fraley et al. (1983) Proc. Natl. Acad. Sci. USA 80:4803-7), direct DNA uptake, and microprojectile bombardment (Klein et al. (1987) Nature 327:70).

The term “marker” refers to a gene or sequence whose presence or absence conveys a detectable phenotype to the host cell or organism. Various types of markers include, but are not limited to, selection markers, screening markers and molecular markers.

The term “selectable markers” refers to markers that are genes. These genes can be expressed to convey a phenotype that makes an organism resistant or susceptible to a specific set of environmental conditions. Screening markers can also convey a phenotype that is a readily observable and distinguishable trait, such as Green Fluorescent Protein (GFP), GUS or beta-galactosidase. Molecular markers are, for example, sequence features that can be uniquely identified by oligonucleotide probing, for example RFLP (restriction fragment length polymorphism), or SSR markers (simple sequence repeat).

The term “vector” or “plasmid” refers to an exogenous, self-replicating nucleic acid molecule that can be introduced into a cell, thereby producing a transformed cell. A vector can include nucleic acid sequences that permit it to replicate in the host cell, such as an origin of replication. Examples include, but are not limited to, a plasmid, cosmid, bacteriophage, or virus that carries exogenous DNA into a cell. A vector can also include one or more genes, antisense molecules, and/or selectable marker genes and other genetic elements known in the art. A vector can transduce, transform, or infect a cell, thereby causing the cell to express the nucleic acid molecules and/or proteins encoded by the vector. A vector optionally includes materials to aid in achieving entry of the nucleic acid molecule into the cell (e.g., a liposome, protein coding, etc.).

The term “donor” or “donor construct” refers to the entire set of DNA segments to be introduced into the host cell or organism as a functional group.

The term “flank” or “flanking” as used herein indicates that the same, similar, or related sequences exist on either side of a given sequence. Segments described as “flanking” are not necessarily directly fused to the segment they flank, as there can be intervening, non-specified DNA between a given sequence and its flanking sequences. These and other terms used to describe relative position are used according to normal accepted usage in the field of genetics.

The term “cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

The term “homologous” in the context of a pair of homologous chromosomes refers to a pair of chromosomes from an individual that are similar in length, gene position and centromere location, and that line up and synapse during meiosis. In an individual, one chromosome of a pair of homologous chromosomes comes from the mother of the individual (i.e., is “maternally-derived”), whereas the other chromosomes of the pair comes from the father (i.e., is “paternally-derived”). In the context of genes, the term “homologous” refers to a pair of genes where each gene resides within each homologous chromosome at the same position and has the same function.

The term “zinc finger nuclease” or “ZFN” refers to a chimeric protein molecule comprising at least one zinc finger DNA binding domain effectively linked to at least one nuclease capable of cleaving DNA. Ordinarily, cleavage by a ZFN at a target locus results in a double stranded break (DSB) at that locus.

The term “zinc finger DNA binding protein”, or “zinc finger protein” refers to a zinc finger DNA binding protein, ZFP, (or binding domain) that is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Zinc finger binding domains may be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; 6,534,261; and 6,785,613; see, also WO 98153058; WO 98153059; WO 98153060; WO 021016536 and WO 031016496; and U.S. Pat. Nos. 6,746,838; 6,866,997; and 7,030,215.

The term “target” or “target locus” or “target region” refers to the gene or DNA segment selected for modification by the targeted genetic recombination method of the present invention. Ordinarily, the target is an endogenous gene, coding segment, control region, intron, exon or portion thereof, of the host organism. However, the target can be any part or parts of the host DNA including an exogenous sequence that was integrated within the nuclear, mitochondrial, or chloroplast genome of the host DNA.

The term “viable” refers to a plant that is capable of normal growth and development.

The term “locus” as used herein refers to a specific physical position on a chromosome or a nucleic acid molecule. Alleles of a locus are located at identical sites on homologous chromosomes. “Loci” the plural of “locus” as used herein refers to a specific physical position on either the same or a different chromosome as well as either the same or a different specific physical position on the nucleic acid molecule.

The term “plurality” refers in a non-limiting manner to any integer equal or greater than one. In this regard, the terms “plurality” and “a plurality” as used herein may include, for example, “single” “multiple” or “one or more”. The terms “plurality” or “a plurality” may be used throughout the specification to describe one or more components, devices, elements, units, parameters, or the like.

The term “recognition sequence” refers to a polynucleotide sequence (either endogenous or exogenous) that is recognized and bound by a site specific nuclease. Typically, this is a DNA sequence within the genome at which a double-strand break is induced in the plant cell genome by a double-strand break inducing agent. The terms “recognition sequence” and “recognition site” are used interchangeably herein.

The term “crossing” refers to the act of fusing gametes via pollination to produce progeny.

The term “transmitting” refers to the introgression or insertion of a desired transgene to at least one progeny plant via a sexual cross between two parent plants, at least one of the parent plants having the desired allele within its genome.

The term “linked”, “tightly linked, and “extremely tightly linked” refers to the linkage between genes or markers, and further refers to the phenomenon in which genes or markers on a chromosome show a measurable probability of being passed on together to individuals in the next generation. The closer two genes or markers are to each other, the closer to (1) this probability becomes. Thus, the term “linked” may refer to one or more genes or markers that are passed together with a gene with a probability greater than 0.5 (which is expected from independent assortment where markers/genes are located on different chromosomes). Because the proximity of two genes or markers on a chromosome is directly related to the probability that the genes or markers will be passed together to individuals in term next generation, the term “linked” may also refer herein to one or more genes or markers that are located within about 0.1 Mb to about 2.0 Mb of one another on the same chromosome. Thus, two “linked” genes or markers may be separated by about 2.00 Mb; about 1.95 Mb; about 1.90 Mb; about 1.85 Mb; about 1.80 Mb; about 1.75 Mb; about 1.70 Mb; about 1.65 Mb; about 1.60 Mb; about 1.55 Mb; about 1.50 Mb; about 1.45 Mb; about 1.40 Mb; about 1.35 Mb; about 1.30 Mb; about 1.25 Mb; about 1.20 Mb; about 1.15 Mb; about 1.10 Mb; about 1.05 Mb; about 1.00 Mb; about 0.95 Mb; about 0.90 Mb; about 0.85 Mb; about 0.80 Mb; about 0.75 Mb; about 0.70 Mb; about 0.65 Mb; about 0.60 Mb; about 0.55 Mb; about 0.50 Mb; about 0.45 Mb; about 0.40 Mb; about 0.35 Mb; about 0.30 Mb; about 0.25 Mb; about 0.20 Mb; about 0.15 Mb; about 0.10 Mb; about 0.05 Mb; about 0.025 Mb; about 0.0125 Mb; and about 0.01 Mb.

The term “unlinked” refers to the lack of physical linkage of transgenic cassettes such that they do not co-segregate in progeny.

The term “homozygous” refers to an organism is said to be homozygous when it has a pair of identical alleles at a corresponding chromosomal locus.

The term “heterozygous” refers to an organism is heterozygous when it has a pair of different alleles at a corresponding chromosomal locus.

### Embodiments

The subject disclosure relates to a method for inserting a donor DNA within a plant genomic target locus. In embodiments, the donor DNA is initially integrated within the plant genome and is then mobilized into a specific plant genomic target locus. In some embodiments, a first viable plant containing a genomic DNA is provided that contains a donor DNA flanked by a plurality of recognition sequences and the plant genomic target locus, wherein the plant genomic target locus also contains at least one recognition sequence. In some embodiments, a second viable plant containing a site specific nuclease is provided. In some embodiments, the first and second viable plants are crossed to produce F1 seed. In some embodiments, the site specific nuclease is expressed and cleaves at least one site specific nuclease recognition sequence to release a donor polynucleotide and to create a double strand break within the plant genomic locus. In some embodiments, the donor DNA is integrated within the plant genomic locus. In some embodiments, the donor DNA is integrated within the plant genomic locus via a non-homologous end joining mechanism.

In an embodiment, the donor DNA is a polynucleotide fragment. Such a polynucleotide fragment contains deoxyribonucleotide base pairs. However, in other embodiments the donor polynucleotide is a donor RNA polynucleotide, containing ribonucleotide base pairs. In further embodiments, the donor polynucleotides are either double stranded or single stranded. The ends of a double stranded donor polynucleotide are either perfectly blunt or contain protruding 5′ or 3′ overhangs (i.e., “sticky ends”). In subsequent embodiments, the donor polynucleotide fragment does not contain regions of homology (i.e., more than 12 base pairs of identical sequence) to any other polynucleotide sequence (i.e., endogenous or exogenous sequence) within the plant genome. In an embodiment, the donor DNA is a polynucleotide fragment that does not encode a coding sequence and does not produce a protein. In other embodiments, the donor DNA is a polynucleotide fragment that does encode an open reading frame, but is not translated into a functional protein (e.g., RNAi molecules). In other embodiments, the donor DNA is a polynucleotide fragment that does encode an open reading frame that can be translated into a functional protein by regulatory expression elements (e.g., promoters, 5′ UTR, intron, 3′UTR, etc.). Non-limiting examples of functional proteins that are encoded by the donor DNA polynucleotide fragment include; selectable markers, agronomic traits, herbicide tolerance traits, insect resistance traits, etc. In further embodiments, the donor DNA polynucleotide fragment encodes a regulatory region or a structural nucleic acid. The donor sequence can be of any length, for example between 2 and 20,000 base pairs in length (or any integer value there between or there above). As provided in this disclosure the donor polynucleotide is stably integrated within the chromosome of a plant, and then subsequently released and targeted into a genomic locus located on a chromosome of the same plant.

In an embodiment the subject disclosure relates to a site specific nuclease that is engineered to cleave a recognition sequence. Site specific nucleases, such as ZFNs, TALENs, meganucleases, and/or CRISPR/CAS, can be engineered to bind and cleave any polynucleotide sequence in the target locus.

In an embodiment, the plant genomic target locus is genomic polynucleotide sequence within the plant genome. In some embodiments the plant genomic target locus is located within a transgene that was stably integrated within the plant genome via a plant transformation method. In other embodiments, the plant genomic target locus is located within an artificial chromosome that was previously inserted within the plant nucleus. In further embodiments, the plant genomic target locus is located within the native or endogenous plant genome. Such a plant genomic target locus may be identified within a coding sequence of the plant genome, or in the regulatory elements flanking the coding sequence. In other embodiments the plant genomic target locus may be identified within a non-coding region of the plant genome.

In accordance with one embodiment, a site specific nuclease is used to cleave genomic DNA. Accordingly, the cleavage introduces a double strand break in a targeted genomic locus to facilitate the insertion of a donor DNA (e.g., a nucleic acid of interest). Selection or identification of a recognition sequence within the plant target locus for binding by a site specific nuclease binding domain can be accomplished, for example, according to the methods disclosed in U.S. Pat. No. 6,453,242, the disclosure of which is incorporated herein, which discloses methods for designing zinc finger proteins (ZFPs) to bind to a selected recognition sequence. It will be clear to those skilled in the art that simple visual inspection of a nucleotide sequence can also be used for selection of a target locus. Accordingly, any means for target locus selection can be used in the methods described herein. Furthermore, a recognition sequence may be designed by those skilled in the art and integrated within a plant genome, such a recognition sequence may be desirable for use as a targeted genomic locus.

For ZFP DNA-binding domains, recognition sequences are generally composed of a plurality of adjacent target subsites. A target subsite refers to the sequence, usually either a nucleotide triplet or a nucleotide quadruplet which may overlap by one nucleotide with an adjacent quadruplet that is bound by an individual zinc finger. See, for example, WO 02/077227, the disclosure of which is incorporated herein. A recognition sequence generally has a length of at least 9 nucleotides and, accordingly, is bound by a zinc finger binding domain comprising at least three zinc fingers. However, binding of, for example, a 4-finger binding domain to a 12-nucleotide recognition sequence, a 5-finger binding domain to a 15-nucleotide recognition sequence or a 6-finger binding domain to an 18-nucleotide recognition sequence, is also possible. As will be apparent, binding of larger binding domains (e.g., 7-, 8-, 9-finger and more) to longer recognition sequences is also consistent with the subject disclosure.

In accordance with one embodiment, it is not necessary for a recognition sequence to be a multiple of three nucleotides. In cases in which cross-strand interactions occur (see, e.g., U.S. Pat. No. 6,453,242 and WO 02/077227), one or more of the individual zinc fingers of a multi-finger binding domain can bind to overlapping quadruplet subsites. As a result, a three-finger protein can bind a 10-nucleotide sequence, wherein the tenth nucleotide is part of a quadruplet bound by a terminal finger, a four-finger protein can bind a 13-nucleotide sequence, wherein the thirteenth nucleotide is part of a quadruplet bound by a terminal finger, etc.

The length and nature of amino acid linker sequences between individual zinc fingers in a multi-finger binding domain also affects binding to a target sequence. For example, the presence of a so-called “non-canonical linker”, “long linker” or “structured linker” between adjacent zinc fingers in a multi-finger binding domain can allow those fingers to bind subsites which are not immediately adjacent. Non-limiting examples of such linkers are described, for example, in U.S. Pat. No. 6,479,626 and WO 01/53480. Accordingly, one or more subsites, in a recognition sequence for a zinc finger binding domain, can be separated from each other by 1, 2, 3, 4, 5 or more nucleotides. One non-limiting example would be a four-finger binding domain that binds to a 13-nucleotide recognition sequence comprising, in sequence, two contiguous 3-nucleotide subsites, an intervening nucleotide, and two contiguous triplet subsites.

While DNA-binding polypeptides identified from proteins that exist in nature typically bind to a discrete nucleotide sequence or motif (e.g., a consensus recognition sequence), methods exist and are known in the art for modifying many such DNA-binding polypeptides to recognize a different nucleotide sequence or motif. DNA-binding polypeptides include, for example and without limitation: zinc finger DNA-binding domains; leucine zippers; TALENS; CRIPSP-cas9; CRISPR-cpf1; UPA DNA-binding domains; GAL4; TAL; LexA; a Tet repressor; LacR; and a steroid hormone receptor.

In some examples, a DNA-binding polypeptide is a zinc finger. Individual zinc finger motifs can be designed to target and bind specifically to any of a large range of DNA sites. Canonical Cys2His2 and non-canonical Cys3His1 zinc finger polypeptides bind DNA by inserting an α-helix into the major groove of the target DNA double helix. Recognition of DNA by a zinc finger is modular; each finger contacts primarily three consecutive base pairs in the target, and a few key residues in the polypeptide mediate recognition. By including multiple zinc finger DNA-binding domains in a targeting endonuclease, the DNA-binding specificity of the targeting endonuclease may be further increased (and hence the specificity of any gene regulatory effects conferred thereby may also be increased). See, e.g., Urnov et al. (2005) Nature 435:646-51. Thus, one or more zinc finger DNA-binding polypeptides may be engineered and utilized such that a targeting endonuclease introduced into a host cell interacts with a DNA sequence that is unique within the genome of the host cell. Preferably, the zinc finger protein is non-naturally occurring in that it is engineered to bind to a recognition sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Alternatively, the DNA-binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural recognition sequences. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 20070117128.

As another alternative, the DNA-binding domain may be derived from a leucine zipper protein. Leucine zippers are a class of proteins that are involved in protein-protein interactions in many eukaryotic regulatory proteins that are important transcription factors associated with gene expression. The leucine zipper refers to a common structural motif shared in these transcriptional factors across several kingdoms including animals, plants, yeasts, etc. The leucine zipper is formed by two polypeptides (homodimer or heterodimer) that bind to specific DNA sequences in a manner where the leucine residues are evenly spaced through an α-helix, such that the leucine residues of the two polypeptides end up on the same face of the helix. The DNA binding specificity of leucine zippers can be utilized in the DNA-binding domains disclosed herein.

In some embodiments, the DNA-binding domain of one or more of the nucleases comprises a naturally occurring or engineered (non-naturally occurring) TAL effector DNA binding domain. See, e.g., U.S. Patent Publication No. 20110301073, incorporated by reference in its entirety herein. The plant pathogenic bacteria of the genus Xanthomonas are known to cause many diseases in important crop plants. Pathogenicity of Xanthomonas depends on a conserved type III secretion (T3S) system which injects more than different effector proteins into the plant cell. Among these injected proteins are transcription activator-like (TALEN) effectors which mimic plant transcriptional activators and manipulate the plant transcriptome (see Kay et al., (2007) Science 318:648-651). These proteins contain a DNA binding domain and a transcriptional activation domain. One of the most well characterized TAL-effectors is AvrBs3 from Xanthomonas campestgris pv. Vesicatoria (see Bonas et al., (1989) Mol Gen Genet 218: 127-136 and WO2010079430). TAL-effectors contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al., (2006) J Plant Physiol 163(3): 256-272). In addition, in the phytopathogenic bacteria Ralstonia solanacearum two genes, designated brg11 and hpx17 have been found that are homologous to the AvrBs3 family of Xanthomonas in the R. solanacearum biovar strain GMI1000 and in the biovar 4 strain RS1000 (See Heuer et al., (2007) Appl and Enviro Micro 73(13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas. See, e.g., U.S. Patent Publication No. 20110301073, incorporated by reference in its entirety.

Specificity of these TAL effectors depends on the sequences found in the tandem repeats. The repeated sequence comprises approximately 102 bp and the repeats are typically 91-100% homologous with each other (Bonas et al., ibid). Polymorphism of the repeats is usually located at positions 12 and 13 and there appears to be a one-to-one correspondence between the identity of the hypervariable diresidues at positions 12 and 13 with the identity of the contiguous nucleotides in the TAL-effector's target sequence (see Moscou and Bogdanove, (2009) Science 326:1501 and Boch et al., (2009) Science 326:1509-1512). Experimentally, the natural code for DNA recognition of these TAL-effectors has been determined such that an HD sequence at positions 12 and 13 leads to a binding to cytosine (C), NG binds to T, NI to A, C, G or T, NN binds to A or G, and ING binds to T. These DNA binding repeats have been assembled into proteins with new combinations and numbers of repeats, to make artificial transcription factors that are able to interact with new sequences and activate the expression of a non-endogenous reporter gene in plant cells (Boch et al., ibid). Engineered TAL proteins have been linked to a FokI cleavage half domain to yield a TAL effector domain nuclease fusion (TALEN) exhibiting activity in a yeast reporter assay (plasmid based target).

The CRISPR (Clustered Regularly Interspaced Short Palindromic Repeats)/Cas (CRISPR Associated) nuclease system is a recently engineered nuclease system based on a bacterial system that can be used for genome engineering. It is based on part of the adaptive immune response of many bacteria and Archaea. When a virus or plasmid invades a bacterium, segments of the invader's DNA are converted into CRISPR RNAs (crRNA) by the ‘immune’ response. This crRNA then associates, through a region of partial complementarity, with another type of RNA called tracrRNA to guide the Cas9 nuclease to a region homologous to the crRNA in the target DNA called a “protospacer”. Cas9 cleaves the DNA to generate blunt ends at the DSB at sites specified by a 20-nucleotide guide sequence contained within the crRNA transcript. Cas9 requires both the crRNA and the tracrRNA for site specific DNA recognition and cleavage. This system has now been engineered such that the crRNA and tracrRNA can be combined into one molecule (the “single guide RNA”), and the crRNA equivalent portion of the single guide RNA can be engineered to guide the Cas9 nuclease to target any desired sequence (see Jinek et al (2012) Science 337, p. 816-821, Jinek et al, (2013), eLife 2:e00471, and David Segal, (2013) eLife 2:e00563). In other examples, the crRNA associates with the tracrRNA to guide the Cpf1 nuclease to a region homologous to the crRNA to cleave DNA with staggered ends (see Zetsche, Bernd, et al. Cell 163.3 (2015): 759-771.). Thus, the CRISPR/Cas system can be engineered to create a double-stranded break (DSB) at a desired target in a genome, and repair of the DSB can be influenced by the use of repair inhibitors to cause an increase in error prone repair.

In certain embodiments, the site specific nuclease protein may be a “functional derivative” of a naturally occurring site specific nuclease protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a site specific nuclease protein polypeptide or a fragment thereof include but are not limited to mutants, fusions, covalent modifications of site specific nuclease protein or a fragment thereof. Site specific nuclease protein, which includes zinc fingers, talens, CRISPR cas9, CRISPR cpf1 or a fragment thereof, as well as derivatives of site specific nuclease proteins or a fragment thereof, may be obtainable from a cell or synthesized chemically or by a combination of these two procedures. The cell may be a cell that naturally produces site specific nuclease protein, or a cell that naturally produces site specific nuclease protein and is genetically engineered to produce the endogenous site specific nuclease protein at a higher expression level or to produce a site specific nuclease protein from an exogenously introduced nucleic acid, which nucleic acid encodes a site specific nuclease protein that is same or different from the endogenous site specific nuclease protein. In some case, the cell does not naturally produce the site specific nuclease protein and is genetically engineered to produce a site specific nuclease protein. The site specific nuclease protein is deployed in plant cells by co-expressing the site specific nuclease protein with other domains that impart functionality to the site specific nuclease protein (e.g., guide RNA for CRISPR; wo forms of guide RNAs can be used to facilitate Cas-mediated genome cleavage as disclosed in Le Cong, F., et al., (2013) Science 339(6121):819-823.).

In other embodiments, the DNA-binding domain may be associated with a cleavage (nuclease) domain. For example, homing endonucleases may be modified in their DNA-binding specificity while retaining nuclease function. In addition, zinc finger proteins may also be fused to a cleavage domain to form a zinc finger nuclease (ZFN). The cleavage domain portion of the fusion proteins disclosed herein can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). Non limiting examples of homing endonucleases and meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. One or more of these enzymes (or functional fragments thereof) can be used as a source of cleavage domains and cleavage half-domains.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is FokI. This particular enzyme is active as a dimer. Bitinaite et al. (1998) Proc. Natl. Acad. Sci. USA 95: 10,570-10,575. Accordingly, for the purposes of the present disclosure, the portion of the FokI enzyme used in the disclosed fusion proteins is considered a cleavage half-domain. Thus, for targeted double-stranded cleavage and/or targeted replacement of cellular sequences using zinc finger-FokI fusions, two fusion proteins, each comprising a FokI cleavage half-domain, can be used to reconstitute a catalytically active cleavage domain. Alternatively, a single polypeptide molecule containing a zinc finger binding domain and two FokI cleavage half-domains can also be used. Parameters for targeted cleavage and targeted sequence alteration using zinc finger-FokI fusions are provided elsewhere in this disclosure.

A cleavage domain or cleavage half-domain can be any portion of a protein that retains cleavage activity, or that retains the ability to multimerize (e.g., dimerize) to form a functional cleavage domain. Exemplary Type IIS restriction enzymes are described in International Publication WO 2007/014275, incorporated by reference herein in its entirety.

To enhance cleavage specificity, cleavage domains may also be modified. In certain embodiments, variants of the cleavage half-domain are employed these variants minimize or prevent homodimerization of the cleavage half-domains. Non-limiting examples of such modified cleavage half-domains are described in detail in WO 2007/014275, incorporated by reference in its entirety herein. In certain embodiments, the cleavage domain comprises an engineered cleavage half-domain (also referred to as dimerization domain mutants) that minimize or prevent homodimerization. Such embodiments are known to those of skill the art and described for example in U.S. Patent Publication Nos. 20050064474; 20060188987; 20070305346 and 20080131962, the disclosures of all of which are incorporated by reference in their entireties herein. Amino acid residues at positions 446, 447, 479, 483, 484, 486, 487, 490, 491, 496, 498, 499, 500, 531, 534, 537, and 538 of FokI are all targets for influencing dimerization of the FokI cleavage half-domains.

Additional engineered cleavage half-domains of FokI that form obligate heterodimers can also be used in the ZFNs described herein. Exemplary engineered cleavage half-domains of Fok I that form obligate heterodimers include a pair in which a first cleavage half-domain includes mutations at amino acid residues at positions 490 and 538 of Fok I and a second cleavage half-domain includes mutations at amino acid residues 486 and 499. In one embodiment, a mutation at 490 replaces Glu (E) with Lys (K); the mutation at 538 replaces Isl (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished. See, e.g., U.S. Patent Publication No. 2008/0131962, the disclosure of which is incorporated by reference in its entirety for all purposes. In certain embodiments, the engineered cleavage half-domain comprises mutations at positions 486, 499 and 496 (numbered relative to wild-type FokI), for instance mutations that replace the wild type Gln (Q) residue at position 486 with a Glu (E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In other embodiments, the engineered cleavage half-domain comprises mutations at positions 490, 538 and 537 (numbered relative to wild-type FokI), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In other embodiments, the engineered cleavage half-domain comprises mutations at positions 490 and 537 (numbered relative to wild-type FokI), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively). (See US Patent Publication No. 20110201055). In other embodiments, the engineered cleavage half domain comprises the “Sharkey” and/or “Sharkey” mutations (see Guo et al, (2010) J. Mol. Biol. 400(1):96-107).

Engineered cleavage half-domains described herein can be prepared using any suitable method, for example, by site-directed mutagenesis of wild-type cleavage half-domains (Fok I) as described in U.S. Patent Publication Nos. 20050064474; 20080131962; and 20110201055. Alternatively, nucleases may be assembled in vivo at the nucleic acid recognition sequence using so-called “split-enzyme” technology (see e.g. U.S. Patent Publication No. 20090068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases can be screened for activity prior to use, for example in a yeast-based chromosomal system as described in WO 2009/042163 and 20090068164. Nuclease expression constructs can be readily designed using methods known in the art. See, e.g., United States Patent Publications 20030232410; 20050208489; 20050026157; 20050064474; 20060188987; 20060063231; and International Publication WO 07/014275. Expression of the nuclease may be under the control of a constitutive promoter or an inducible promoter, for example the galactokinase promoter which is activated (de-repressed) in the presence of raffinose and/or galactose and repressed in presence of glucose.

Distance between recognition sequences refers to the number of nucleotides or nucleotide pairs intervening between two recognition sequences as measured from the edges of the sequences nearest each other. In certain embodiments in which cleavage depends on the binding of two zinc finger domain/cleavage half-domain fusion molecules to separate recognition sequences, the two recognition sequences can be on opposite DNA strands. In other embodiments, both recognition sequences are on the same DNA strand. For targeted integration into the optimal genomic locus, one or more ZFPs are engineered to bind a recognition sequence at or near the predetermined cleavage site, and a fusion protein comprising the engineered DNA-binding domain and a cleavage domain is expressed in the cell. Upon binding of the zinc finger portion of the fusion protein to the recognition sequence, the DNA is cleaved, preferably via a double-stranded break, near the recognition sequence by the cleavage domain.

The presence of a double-stranded break in the optimal genomic locus facilitates integration of exogenous sequences via NHEJ. In some instances the presence of a double-stranded break in the optimal genomic locus facilitates integration of exogenous sequences via a combination of NHEJ and HDR. Thus, in one embodiment the polynucleotide comprising the donor DNA to be inserted into the targeted genomic locus will not include regions of homology with the targeted genomic locus. A polynucleotide fragment spanning 12 base pairs of more of identical sequence between the donor DNA and targeted genomic locus are considered as a region of homology for such a purpose.

In some instances the deployment of more than one site specific nuclease protein is provided to the plant cell. In an embodiment, two site specific nuclease proteins may be provided to the plant cell, wherein each site specific nuclease cleaves at a unique location of the genome. In an embodiment, three site specific nuclease proteins may be provided to the plant cell, wherein each site specific nuclease cleaves at a unique location of the genome. In an embodiment, four site specific nuclease proteins may be provided to the plant cell, wherein each site specific nuclease cleaves at a unique location of the genome. In an embodiment, five site specific nuclease proteins may be provided to the plant cell, wherein each site specific nuclease cleaves at a unique location of the genome. In an embodiment, six or more site specific nuclease proteins may be provided to the plant cell, wherein each site specific nuclease cleaves at a unique location of the genome. Such usage of the use of multiple site specific nuclease proteins will be applicable by those with skill in the art

Any of the well-known procedures for introducing polynucleotide donor sequences and nuclease sequences as a DNA construct (e.g., gene expression cassette) into host cells may be used in accordance with the present disclosure. These include the use of calcium phosphate transfection, polybrene, protoplast fusion, PEG, electroporation, ultrasonic methods (e.g., sonoporation), liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular nucleic acid insertion procedure used be capable, of successfully introducing at least one gene into the host cell capable of expressing the protein of choice.

As noted above, DNA constructs may be introduced into the genome of a desired plant species by a variety of conventional techniques. For reviews of such techniques see, for example, Weissbach & Weissbach Methods for Plant Molecular Biology (1988, Academic Press, N.Y.) Section VIII, pp. 421-463; and Grierson & Corey, Plant Molecular Biology (1988, 2d Ed.), Blackie, London, Ch. 7-9. A DNA construct may be introduced directly into the genomic DNA of the plant cell using techniques such as electroporation and microinjection of plant cell protoplasts, by agitation with silicon carbide fibers (see, e.g., U.S. Pat. Nos. 5,302,523 and 5,464,765), or the DNA constructs can be introduced directly to plant tissue using biolistic methods, such as DNA particle bombardment (see, e.g., Klein et al. (1987) Nature 327:70-73). Alternatively, the DNA construct can be introduced into the plant cell via nanoparticle transformation (see, e.g., US Patent Publication No. 20090104700, which is incorporated herein by reference in its entirety). Alternatively, the DNA constructs may be combined with suitable T-DNA border/flanking regions and introduced into a conventional Agrobacterium tumefaciens host vector. Agrobacterium tumefaciens-mediated transformation techniques, including disarming and use of binary vectors, are well described in the scientific literature. See, for example Horsch et al. (1984) Science 233:496-498, and Fraley et al. (1983) Proc. Nat'l. Acad. Sci. USA 80:4803.

In addition, gene transfer may be achieved using non-Agrobacterium bacteria or viruses such as Rhizobium sp. NGR234, Sinorhizoboium meliloti, Mesorhizobium loti, potato virus X, cauliflower mosaic virus and cassava vein mosaic virus and/or tobacco mosaic virus, See, e.g., Chung et al. (2006) Trends Plant Sci. 11(1):1-4. The virulence functions of the Agrobacterium tumefaciens host will direct the insertion of a T-strand containing the construct and adjacent marker into the plant cell DNA when the cell is infected by the bacteria using binary T DNA vector (Bevan (1984) Nuc. Acid Res. 12:8711-8721) or the co-cultivation procedure (Horsch et al. (1985) Science 227:1229-1231). Generally, the Agrobacterium transformation system is used to engineer monocotyledonous plants (Bevan et al. (1982) Ann. Rev. Genet. 16:357-384; Rogers et al. (1986) Methods Enzymol. 118:627-641). The Agrobacterium transformation system may also be used to transform, as well as transfer, DNA to monocotyledonous plants and plant cells. See U.S. Pat. No. 5,591,616; Hernalsteen et al. (1984) EMBO J. 3:3039-3041; Hooykass-Van Slogteren et al. (1984) Nature 311:763-764; Grimsley et al. (1987) Nature 325:1677-179; Boulton et al. (1989) Plant Mol. Biol. 12:31-40; and Gould et al. (1991) Plant Physiol. 95:426-434.

Alternative gene transfer and transformation methods include, but are not limited to, protoplast transformation through calcium-, polyethylene glycol (PEG)- or electroporation-mediated uptake of naked DNA (see Paszkowski et al. (1984) EMBO J. 3:2717-2722, Potrykus et al. (1985) Molec. Gen. Genet. 199:169-177; Fromm et al. (1985) Proc. Nat. Acad. Sci. USA 82:5824-5828; and Shimamoto (1989) Nature 338:274-276) and electroporation of plant tissues (D'Halluin et al. (1992) Plant Cell 4:1495-1505). Additional methods for plant cell transformation include microinjection, silicon carbide mediated DNA uptake (Kaeppler et al. (1990) Plant Cell Reporter 9:415-418), and microprojectile bombardment (see Klein et al. (1988) Proc. Nat. Acad. Sci. USA 85:4305-4309; and Gordon-Kamm et al. (1990) Plant Cell 2:603-618).

In specific embodiments, the donor DNA is integrated within a genomic target locus during a cytological phase. The cell division cycle is normally composed of four distinct phases, which in typical somatic cells take 18-24 hours to complete. The S-phase represents the period when chromosomal DNA is duplicated, this is then followed by a gap phase (G2) where cells prepare to segregate chromosomes between daughter cells during M-phase. After completion of M-phase, cells enter a second gap phase, G1, which separates M- from S-phase. G1 is a cell phase where the cell decides to continue dividing or withdraw from the cell cycle.

In certain embodiments, the frequency of recombination can be enhanced by arresting the cells in the gap 2 (G2) phase of the cell cycle and/or by activating the expression of one or more molecules (protein, RNA) involved in non-homologous end-joining recombination. In certain embodiments, the frequency of recombination can be enhanced by arresting the cells in the gap 2 (G2) phase of the cell cycle and/or by activating the expression of one or more molecules (protein, RNA) involved in non-homologous end-joining recombination and/or by inhibiting the expression or activity of proteins involved in homologous recombination.

In certain embodiments, the frequency of recombination can be enhanced by arresting the cells in the gap 1 (G1) phase of the cell cycle and/or by activating the expression of one or more molecules (protein, RNA) involved in non-homologous end-joining recombination. In certain embodiments, the frequency of recombination can be enhanced by arresting the cells in the gap 1 (G1) phase of the cell cycle and/or by activating the expression of one or more molecules (protein, RNA) involved in non-homologous end-joining recombination and/or by inhibiting the expression or activity of proteins involved in homologous recombination.

In certain embodiments, the frequency of recombination can be enhanced by arresting the cells in the DNA synthesis (S phase) of the cell cycle and/or by activating the expression of one or more molecules (protein, RNA) involved in non-homologous end-joining recombination. In certain embodiments, the frequency of recombination can be enhanced by arresting the cells in the DNA synthesis (S phase) of the cell cycle and/or by activating the expression of one or more molecules (protein, RNA) involved in non-homologous end-joining recombination and/or by inhibiting the expression or activity of proteins involved in homologous recombination.

In certain embodiments, the frequency of recombination can be enhanced by arresting the cells in the mitosis (M) phase of the cell cycle and/or by activating the expression of one or more molecules (protein, RNA) involved in non-homologous end-joining recombination. In certain embodiments, the frequency of recombination can be enhanced by arresting the cells in the mitosis (M) phase of the cell cycle and/or by activating the expression of one or more molecules (protein, RNA) involved in non-homologous end-joining recombination and/or by inhibiting the expression or activity of proteins involved in homologous recombination.

In further embodiments, a trait can include a transgenic trait. Transgenic traits that are suitable for use in the present disclosed constructs include, but are not limited to, coding sequences that confer (1) resistance to pests or disease, (2) tolerance to herbicides, (3) value added agronomic traits, such as; yield improvement, nitrogen use efficiency, water use efficiency, and nutritional quality, (4) binding of a protein to DNA in a site specific manner, (5) expression of small RNA, and (6) selectable markers. In accordance with one embodiment, the transgene encodes a selectable marker or a gene product conferring insecticidal resistance, herbicide tolerance, small RNA expression, nitrogen use efficiency, water use efficiency, or nutritional quality.

### 1. Insect Resistance

Various insect resistance coding sequences are an embodiment of a transgenic trait. Exemplary insect resistance coding sequences are known in the art. As embodiments of insect resistance coding sequences that can be operably linked to the regulatory elements of the subject disclosure, the following traits are provided. Coding sequences that provide exemplary Lepidopteran insect resistance include: cry1A; cry1A.105; cry1Ab; cry1Ab (truncated); cry1Ab-Ac (fusion protein); cry1Ac (marketed as Widestrike®); cry1C; cry1F (marketed as Widestrike®); cry1Fa2; cry2Ab2; cry2Ae; cry9C; mocry1F; pinII (protease inhibitor protein); vip3A(a); and vip3Aa20. Coding sequences that provide exemplary Coleopteran insect resistance include: cry34Ab1 (marketed as Herculex®); cry35Ab1 (marketed as Herculex®); cry3A; cry3Bb1; dvsnf7; and mcry3A. Coding sequences that provide exemplary multi-insect resistance include ecry31.Ab. The above list of insect resistance genes is not meant to be limiting. Any insect resistance genes are encompassed by the present disclosure.

### 2. Herbicide Tolerance

Various herbicide tolerance coding sequences are an embodiment of a transgenic trait. Exemplary herbicide tolerance coding sequences are known in the art. As embodiments of herbicide tolerance coding sequences that can be operably linked to the regulatory elements of the subject disclosure, the following traits are provided. The glyphosate herbicide contains a mode of action by inhibiting the EPSPS enzyme (5-enolpyruvylshikimate-3-phosphate synthase). This enzyme is involved in the biosynthesis of aromatic amino acids that are essential for growth and development of plants. Various enzymatic mechanisms are known in the art that can be utilized to inhibit this enzyme. The genes that encode such enzymes can be operably linked to the gene regulatory elements of the subject disclosure. In an embodiment, selectable marker genes include, but are not limited to genes encoding glyphosate resistance genes include: mutant EPSPS genes such as 2mEPSPS genes, cp4 EPSPS genes, mEPSPS genes, dgt-28 genes; aroA genes; and glyphosate degradation genes such as glyphosate acetyl transferase genes (gat) and glyphosate oxidase genes (gox). These traits are currently marketed as Gly-Tol™, Optimum® GAT®, Agrisure® GT and Roundup Ready®. Resistance genes for glufosinate and/or bialaphos compounds include dsm-2, bar and pat genes. The bar and pat traits are currently marketed as LibertyLink®. Also included are tolerance genes that provide resistance to 2,4-D such as aad-1 genes (it should be noted that aad-1 genes have further activity on arloxyphenoxypropionate herbicides) and aad-12 genes (it should be noted that aad-12 genes have further activity on pyidyloxyacetate synthetic auxins). These traits are marketed as Enlist® crop protection technology. Resistance genes for ALS inhibitors (sulfonylureas, imidazolinones, triazolopyrimidines, pyrimidinylthiobenzoates, and sulfonylamino-carbonyl-triazolinones) are known in the art. These resistance genes most commonly result from point mutations to the ALS encoding gene sequence. Other ALS inhibitor resistance genes include hra genes, the csr1-2 genes, Sr-HrA genes, and surB genes. Some of the traits are marketed under the tradename Clearfield®. Herbicides that inhibit HPPD include the pyrazolones such as pyrazoxyfen, benzofenap, and topramezone; triketones such as mesotrione, sulcotrione, tembotrione, benzobicyclon; and diketonitriles such as isoxaflutole. These exemplary HPPD herbicides can be tolerated by known traits. Examples of HPPD inhibitors include hppdPF_W336 genes (for resistance to isoxaflutole) and avhppd-03 genes (for resistance to meostrione). An example of oxynil herbicide tolerant traits include the bxn gene, which has been showed to impart resistance to the herbicide/antibiotic bromoxynil. Resistance genes for dicamba include the dicamba monooxygenase gene (dmo) as disclosed in International PCT Publication No. WO 2008/105890. Resistance genes for PPO or PROTOX inhibitor type herbicides (e.g., acifluorfen, butafenacil, flupropazil, pentoxazone, carfentrazone, fluazolate, pyraflufen, aclonifen, azafenidin, flumioxazin, flumiclorac, bifenox, oxyfluorfen, lactofen, fomesafen, fluoroglycofen, and sulfentrazone) are known in the art. Exemplary genes conferring resistance to PPO include over expression of a wild-type Arabidopsis thaliana PPO enzyme (Lermontova I and Grimm B, (2000) Overexpression of plastidic protoporphyrinogen IX oxidase leads to resistance to the diphenyl-ether herbicide acifluorfen. Plant Physiol 122:75-83.), the B. subtilis PPO gene (Li, X. and Nicholl D. 2005. Development of PPO inhibitor-resistant cultures and crops. Pest Manag. Sci. 61:277-285 and Choi K W, Han O, Lee H J, Yun Y C, Moon Y H, Kim MK, Kuk Y I, Han S U and Guh J O, (1998) Generation of resistance to the diphenyl ether herbicide, oxyfluorfen, via expression of the Bacillus subtilis protoporphyrinogen oxidase gene in transgenic tobacco plants. Biosci Biotechnol Biochem 62:558-560.) Resistance genes for pyridinoxy or phenoxy proprionic acids and cyclohexones include the ACCase inhibitor-encoding genes (e.g., Acc1-S1, Acc1-S2 and Acc1-S3). Exemplary genes conferring resistance to cyclohexanediones and/or aryloxyphenoxypropanoic acid include haloxyfop, diclofop, fenoxyprop, fluazifop, and quizalofop. Finally, herbicides can inhibit photosynthesis, including triazine or benzonitrile are provided tolerance by psbA genes (tolerance to triazine), 1s+ genes (tolerance to triazine), and nitrilase genes (tolerance to benzonitrile). The above list of herbicide tolerance genes is not meant to be limiting. Any herbicide tolerance genes are encompassed by the present disclosure.

### 3. Agronomic Traits

Various agronomic trait coding sequences are an embodiment of a transgenic trait. Exemplary agronomic trait coding sequences are known in the art. As embodiments of agronomic trait coding sequences that can be operably linked to the regulatory elements of the subject disclosure, the following traits are provided. Delayed fruit softening as provided by the pg genes inhibit the production of polygalacturonase enzyme responsible for the breakdown of pectin molecules in the cell wall, and thus causes delayed softening of the fruit. Further, delayed fruit ripening/senescence of acc genes act to suppress the normal expression of the native acc synthase gene, resulting in reduced ethylene production and delayed fruit ripening. Whereas, the accd genes metabolize the precursor of the fruit ripening hormone ethylene, resulting in delayed fruit ripening. Alternatively, the sam-k genes cause delayed ripening by reducing S-adenosylmethionine (SAM), a substrate for ethylene production. Drought stress tolerance phenotypes as provided by cspB genes maintain normal cellular functions under water stress conditions by preserving RNA stability and translation. Another example includes the EcBetA genes that catalyze the production of the osmoprotectant compound glycine betaine conferring tolerance to water stress. In addition, the RmBetA genes catalyze the production of the osmoprotectant compound glycine betaine conferring tolerance to water stress. Photosynthesis and yield enhancement is provided with the bbx32 gene that expresses a protein that interacts with one or more endogenous transcription factors to regulate the plant's day/night physiological processes. Ethanol production can be increase by expression of the amy797E genes that encode a thermostable alpha-amylase enzyme that enhances bioethanol production by increasing the thermostability of amylase used in degrading starch. Finally, modified amino acid compositions can result by the expression of the cordapA genes that encode a dihydrodipicolinate synthase enzyme that increases the production of amino acid lysine. The above list of agronomic trait coding sequences is not meant to be limiting. Any agronomic trait coding sequence is encompassed by the present disclosure.

### 4. DNA Binding Proteins

Various DNA binding protein coding sequences are an embodiment of a transgenic trait. Exemplary DNA binding protein coding sequences are known in the art. As embodiments of DNA binding protein coding sequences that can be operably linked to the regulatory elements of the subject disclosure, the following types of DNA binding proteins can include; Zinc Fingers, Talens, CRISPRS, and meganucleases. The above list of DNA binding protein coding sequences is not meant to be limiting. Any DNA binding protein coding sequences is encompassed by the present disclosure.

### 5. Small RNA

Various small RNAs are an embodiment of a transgenic trait. Exemplary small RNA traits are known in the art. As embodiments of small RNA coding sequences that can be operably linked to the regulatory elements of the subject disclosure, the following traits are provided. For example, delayed fruit ripening/senescence of the anti-efe small RNA delays ripening by suppressing the production of ethylene via silencing of the ACO gene that encodes an ethylene-forming enzyme. The altered lignin production of ccomt small RNA reduces content of guanacyl (G) lignin by inhibition of the endogenous S-adenosyl-L-methionine: trans-caffeoyl CoA 3-O-methyltransferase (CCOMT gene). Further, the Black Spot Bruise Tolerance in Solanum verrucosum can be reduced by the Ppo5 small RNA which triggers the degradation of Ppo5 transcripts to block black spot bruise development. Also included is the dvsnf7 small RNA that inhibits Western Corn Rootworm with dsRNA containing a 240 bp fragment of the Western Corn Rootworm Snf7 gene. Modified starch/carbohydrates can result from small RNA such as the pPhL small RNA (degrades PhL transcripts to limit the formation of reducing sugars through starch degradation) and pR1 small RNA (degrades R1 transcripts to limit the formation of reducing sugars through starch degradation). Additional, benefits such as reduced acrylamide resulting from the asn1 small RNA that triggers degradation of Asn1 to impair asparagine formation and reduce polyacrylamide. Finally, the non-browning phenotype of pgas ppo suppression small RNA results in suppressing PPO to produce apples with a non-browning phenotype. The above list of small RNAs is not meant to be limiting. Any small RNA encoding sequences are encompassed by the present disclosure.

### 6. Selectable Markers

Various selectable markers also described as reporter genes are an embodiment of a transgenic trait. Many methods are available to confirm expression of selectable markers in transformed plants, including for example DNA sequencing and PCR (polymerase chain reaction), Southern blotting, RNA blotting, immunological methods for detection of a protein expressed from the vector. But, usually the reporter genes are observed through visual observation of proteins that when expressed produce a colored product. Exemplary reporter genes are known in the art and encode β-glucuronidase (GUS), luciferase, green fluorescent protein (GFP), yellow fluorescent protein (YFP, Phi-YFP), red fluorescent protein (DsRFP, RFP, etc), β-galactosidase, and the like (See Sambrook, et al., Molecular Cloning: A Laboratory Manual, Third Edition, Cold Spring Harbor Press, N.Y., 2001, the content of which is incorporated herein by reference in its entirety).

Selectable marker genes are utilized for selection of transformed cells or tissues. Selectable marker genes include genes encoding antibiotic resistance, such as those encoding neomycin phosphotransferase II (NEO), spectinomycin/streptinomycin resistance (AAD), and hygromycin phosphotransferase (HPT or HGR) as well as genes conferring resistance to herbicidal compounds. Herbicide resistance genes generally code for a modified target protein insensitive to the herbicide or for an enzyme that degrades or detoxifies the herbicide in the plant before it can act. For example, resistance to glyphosate has been obtained by using genes coding for mutant target enzymes, 5-enolpyruvylshikimate-3-phosphate synthase (EPSPS). Genes and mutants for EPSPS are well known, and further described below. Resistance to glufosinate ammonium, bromoxynil, and 2,4-dichlorophenoxyacetate (2,4-D) have been obtained by using bacterial genes encoding PAT or DSM-2, a nitrilase, an AAD-1, or an AAD-12, each of which are examples of proteins that detoxify their respective herbicides.

In an embodiment, herbicides can inhibit the growing point or meristem, including imidazolinone or sulfonylurea, and genes for resistance/tolerance of acetohydroxyacid synthase (AHAS) and acetolactate synthase (ALS) for these herbicides are well known. Glyphosate resistance genes include mutant 5-enolpyruvylshikimate-3-phosphate synthase (EPSPs) and dgt-28 genes (via the introduction of recombinant nucleic acids and/or various forms of in vivo mutagenesis of native EPSPs genes), aroA genes and glyphosate acetyl transferase (GAT) genes, respectively). Resistance genes for other phosphono compounds include bar and pat genes from Streptomyces species, including Streptomyces hygroscopicus and Streptomyces viridichromogenes, and pyridinoxy or phenoxy proprionic acids and cyclohexones (ACCase inhibitor-encoding genes). Exemplary genes conferring resistance to cyclohexanediones and/or aryloxyphenoxypropanoic acid (including haloxyfop, diclofop, fenoxyprop, fluazifop, quizalofop) include genes of acetyl coenzyme A carboxylase (ACCase); Acc1-S1, Acc1-S2 and Acc1-S3. In an embodiment, herbicides can inhibit photosynthesis, including triazine (psbA and 1s+ genes) or benzonitrile (nitrilase gene). Furthermore, such selectable markers can include positive selection markers such as phosphomannose isomerase (PMI) enzyme.

In an embodiment, selectable marker genes include, but are not limited to genes encoding: 2,4-D; neomycin phosphotransferase II; cyanamide hydratase; aspartate kinase; dihydrodipicolinate synthase; tryptophan decarboxylase; dihydrodipicolinate synthase and desensitized aspartate kinase; bar gene; tryptophan decarboxylase; neomycin phosphotransferase (NEO); hygromycin phosphotransferase (HPT or HYG); dihydrofolate reductase (DHFR); phosphinothricin acetyltransferase; 2,2-dichloropropionic acid dehalogenase; acetohydroxyacid synthase; 5-enolpyruvyl-shikimate-phosphate synthase (aroA); haloarylnitrilase; acetyl-coenzyme A carboxylase; dihydropteroate synthase (sul I); and 32 kD photosystem II polypeptide (psbA). An embodiment also includes selectable marker genes encoding resistance to: chloramphenicol; methotrexate; hygromycin; spectinomycin; bromoxynil; glyphosate; and phosphinothricin. The above list of selectable marker genes is not meant to be limiting. Any reporter or selectable marker gene are encompassed by the present disclosure.

In some embodiments the coding sequences are synthesized for optimal expression in a plant. For example, in an embodiment, a coding sequence of a gene has been modified by codon optimization to enhance expression in plants. An insecticidal resistance transgene, an herbicide tolerance transgene, a nitrogen use efficiency transgene, a water use efficiency transgene, a nutritional quality transgene, a DNA binding transgene, or a selectable marker transgene can be optimized for expression in a particular plant species or alternatively can be modified for optimal expression in dicotyledonous or monocotyledonous plants. Plant preferred codons may be determined from the codons of highest frequency in the proteins expressed in the largest amount in the particular plant species of interest. In an embodiment, a coding sequence, gene, or transgene is designed to be expressed in plants at a higher level resulting in higher transformation efficiency. Methods for plant optimization of genes are well known. Guidance regarding the optimization and production of synthetic DNA sequences can be found in, for example, WO2013016546, WO2011146524, WO1997013402, U.S. Pat. No. 6,166,302, and U.S. Pat. No. 5,380,831, herein incorporated by reference.

In further embodiments, a trait can include a non-transgenic trait, such as a native trait or an endogenous trait. Exemplary native traits can include yield traits, resistance to disease traits, resistance to pests traits, tolerance to herbicide tolerance traits, growth traits, size traits, production of biomass traits, amount of produced seeds traits, resistance against salinity traits, resistance against heat stress traits, resistance against cold stress traits, resistance against drought stress traits, male sterility traits, waxy starch traits, modified fatty acid metabolism traits, modified phytic acid metabolism traits, modified carbohydrate metabolism traits, modified protein metabolism traits, and any combination of such traits.

In further embodiments, exemplary native traits can include early vigor, stress tolerance, drought tolerance, increased nutrient use efficiency, increased root mass and increased water use efficiency. Additional exemplary native traits can include resistance to fungal, bacterial and viral pathogens, plant insect resistance; modified flower size, modified flower number, modified flower pigmentation and shape, modified leaf number, modified leaf pigmentation and shape, modified seed number, modified pattern or distribution of leaves and flowers, modified stem length between nodes, modified root mass and root development characteristics, and increased drought, salt and antibiotic tolerance. Fruit-specific native traits include modified lycopene content, modified content of metabolites derived from lycopene including carotenes, anthocyanins and xanthophylls, modified vitamin A content, modified vitamin C content, modified vitamin E content, modified fruit pigmentation and shape, modified fruit ripening characteristics, fruit resistance to fungal, bacterial and viral pathogens, fruit resistance to insects, modified fruit size, and modified fruit texture, e.g., soluble solids, total solids, and cell wall components.

In an aspect, the native traits may be specific to a particular crop. Exemplary native traits in corn can include the traits described in U.S. Pat. No. 9,288,955, herein incorporated by reference in its entirety. Exemplary native traits in soybean can include the traits described in U.S. Pat. No. 9,313,978, herein incorporated by reference in its entirety. Exemplary native traits in cotton can include the traits described in U.S. Pat. No. 8,614,375, herein incorporated by reference in its entirety. Exemplary native traits in sorghum can include the traits described in U.S. Pat. No. 9,080,182, herein incorporated by reference in its entirety. Exemplary native traits in wheat can include the traits described in U.S. Patent Application No. 2015/0040262, herein incorporated by reference in its entirety. Exemplary native traits in wheat can include the traits described in U.S. Pat. No. 8,927,833, herein incorporated by reference in its entirety. Exemplary native traits in Brassica plants can include the traits described in U.S. Pat. No. 8,563,810, herein incorporated by reference in its entirety. Exemplary native traits in tobacco plants can include the traits described in U.S. Pat. No. 9,096,864, herein incorporated by reference in its entirety.

Means of confirming the integration of a transgene or transgenic trait are known in the art. For example the detection of the transgene or transgenic trait can be achieved, for example, by the polymerase chain reaction (PCR). The PCR detection is done by the use of two oligonucleotide primers flanking the polymorphic segment of the polymorphism followed by DNA amplification. This step involves repeated cycles of heat denaturation of the DNA followed by annealing of the primers to their complementary sequences at low temperatures, and extension of the annealed primers with DNA polymerase. Size separation of DNA fragments on agarose or polyacrylamide gels following amplification, comprises the major part of the methodology. Such selection and screening methodologies are well known to those skilled in the art. Molecular confirmation methods that can be used to identify transgenic plants are known to those with skill in the art. Several exemplary methods are further described below.

Molecular Beacons have been described for use in sequence detection. Briefly, a FRET oligonucleotide probe is designed that overlaps the flanking genomic and insert DNA junction. The unique structure of the FRET probe results in it containing a secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe(s) to the target sequence results in the removal of the probe secondary structure and spatial separation of the fluorescent and quenching moieties. A fluorescent signal indicates the presence of the flanking genomic/transgene insert sequence due to successful amplification and hybridization. Such a molecular beacon assay for detection of as an amplification reaction is an embodiment of the subject disclosure.

Hydrolysis probe assay, otherwise known as TAQMAN® (Life Technologies, Foster City, Calif.), is a method of detecting and quantifying the presence of a DNA sequence. Briefly, a FRET oligonucleotide probe is designed with one oligo within the transgene and one in the flanking genomic sequence for event-specific detection. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization. Such a hydrolysis probe assay for detection of as an amplification reaction is an embodiment of the subject disclosure.

KASPar® assays are a method of detecting and quantifying the presence of a DNA sequence. Briefly, the genomic DNA sample comprising the integrated gene expression cassette polynucleotide is screened using a polymerase chain reaction (PCR) based assay known as a KASPar® assay system. The KASPar® assay used in the practice of the subject disclosure can utilize a KASPar® PCR assay mixture which contains multiple primers. The primers used in the PCR assay mixture can comprise at least one forward primers and at least one reverse primer. The forward primer contains a sequence corresponding to a specific region of the DNA polynucleotide, and the reverse primer contains a sequence corresponding to a specific region of the genomic sequence. In addition, the primers used in the PCR assay mixture can comprise at least one forward primers and at least one reverse primer. For example, the KASPar® PCR assay mixture can use two forward primers corresponding to two different alleles and one reverse primer. One of the forward primers contains a sequence corresponding to specific region of the endogenous genomic sequence. The second forward primer contains a sequence corresponding to a specific region of the DNA polynucleotide. The reverse primer contains a sequence corresponding to a specific region of the genomic sequence. Such a KASPar® assay for detection of an amplification reaction is an embodiment of the subject disclosure.

In some embodiments the fluorescent signal or fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye.

In other embodiments the amplification reaction is run using suitable second fluorescent DNA dyes that are capable of staining cellular DNA at a concentration range detectable by flow cytometry, and have a fluorescent emission spectrum which is detectable by a real time thermocycler. It should be appreciated by those of ordinary skill in the art that other nucleic acid dyes are known and are continually being identified. Any suitable nucleic acid dye with appropriate excitation and emission spectra can be employed, such as YO-PRO-1®, SYTOX Green®, SYBR Green I®, SYTO11®, SYTO12®, SYTO13®, BOBO®, YOYO®, and TOTO®.

In further embodiments, Next Generation Sequencing (NGS) can be used for detection. As described by Brautigma et al., 2010, DNA sequence analysis can be used to determine the nucleotide sequence of the isolated and amplified fragment. The amplified fragments can be isolated and sub-cloned into a vector and sequenced using chain-terminator method (also referred to as Sanger sequencing) or Dye-terminator sequencing. In addition, the amplicon can be sequenced with Next Generation Sequencing. NGS technologies do not require the sub-cloning step, and multiple sequencing reads can be completed in a single reaction. Three NGS platforms are commercially available, the Genome Sequencer FLX™ from 454 Life Sciences/Roche, the Illumina Genome Analyser™ from Solexa and Applied Biosystems' SOLiD™ (acronym for: ‘Sequencing by Oligo Ligation and Detection’). In addition, there are two single molecule sequencing methods that are currently being developed. These include the true Single Molecule Sequencing (tSMS) from Helicos Bioscience™ and the Single Molecule Real Time™ sequencing (SMRT) from Pacific Biosciences.

The Genome Sequencher FLX™ which is marketed by 454 Life Sciences/Roche is a long read NGS, which uses emulsion PCR and pyrosequencing to generate sequencing reads. DNA fragments of 300-800 bp or libraries containing fragments of 3-20 kb can be used. The reactions can produce over a million reads of about 250 to 400 bases per run for a total yield of 250 to 400 megabases. This technology produces the longest reads but the total sequence output per run is low compared to other NGS technologies.

The Illumina Genome Analyser™ which is marketed by Solexa™ is a short read NGS which uses sequencing by synthesis approach with fluorescent dye-labeled reversible terminator nucleotides and is based on solid-phase bridge PCR. Construction of paired end sequencing libraries containing DNA fragments of up to 10 kb can be used. The reactions produce over 100 million short reads that are 35-76 bases in length. This data can produce from 3-6 gigabases per run.

The Sequencing by Oligo Ligation and Detection (SOLiD) system marketed by Applied Biosystems™ is a short read technology. This NGS technology uses fragmented double stranded DNA that are up to 10 kb in length. The system uses sequencing by ligation of dye-labelled oligonucleotide primers and emulsion PCR to generate one billion short reads that result in a total sequence output of up to 30 gigabases per run.

tSMS of Helicos Bioscience™ and SMRT of Pacific Biosciences™ apply a different approach which uses single DNA molecules for the sequence reactions. The tSMS Helicos™ system produces up to 800 million short reads that result in 21 gigabases per run. These reactions are completed using fluorescent dye-labelled virtual terminator nucleotides that is described as a ‘sequencing by synthesis’ approach.

The SMRT Next Generation Sequencing system marketed by Pacific Biosciences™ uses a real time sequencing by synthesis. This technology can produce reads of up to 1,000 bp in length as a result of not being limited by reversible terminators. Raw read throughput that is equivalent to one-fold coverage of a diploid human genome can be produced per day using this technology.

An embodiment of the subject disclosure provides a method for transmitting a transgene into other plants, by:

a) crossing a first plant regenerated from a plant cell or tissue transformed with an isolated nucleic acid molecule comprising a genomic target locus and the transgene with a second plant regenerated from a plant cell or tissue transformed with an isolated nucleic acid molecule comprising a promoter operably linked to a zinc finger nuclease;
b) expressing the zinc finger nuclease so that a first zinc finger nuclease monomer is paired with a second zinc finger nuclease monomer;
c) obtaining a F1 plant resulting from the cross wherein the transgene is specifically and stably integrated within the genomic target locus via non-homologous end joining; and
d) cultivating the F1 plant resulting from the cross.

In yet another aspect of the subject disclosure, processes are provided for producing a progeny of first generation (F1) plants, which processes generally comprise crossing a first parent plant with a second parent plant wherein the first parent plant or the second parent plant comprise a donor DNA flanked by recognition sequences and/or a site specific nuclease. Any time the first parent plant is crossed with a second parent plant, wherein the second parent plant is different (i.e., contains transgenes not present in the first parent plant) from the first parent plant, a progeny or first generation (F1) corn hybrid plant is produced. As such, a progeny or F1 hybrid plant may be produced by the methods and compositions of the subject disclosure. Therefore, any progeny or F1 plant or seed which is produced wherein the donor DNA is integrated within the target genomic locus via a non-homologous end joining cellular repair mechanism is an embodiment of the subject disclosure.

In embodiments of the present disclosure, the step of “crossing” a first and second plant comprises planting, in pollinating proximity, seeds of a first plant and a second, plant. In some instances the step of “crossing” a first and second plant comprises emasculating a first parent plant and applying pollen obtained from a second plant to the stigma of the first plant to fertilize the first plant. If the parental plants differ in timing of sexual maturity, techniques may be employed to obtain an appropriate nick, i.e., to ensure the availability of pollen from the parent plant designated the male during the time at which silks on the parent plant designated the female are receptive to the pollen. Methods that may be employed to obtain the desired nick include delaying the flowering of the faster maturing plant, such as, but not limited to delaying the planting of the faster maturing seed, cutting or burning the top leaves of the faster maturing plant (without killing the plant) or speeding up the flowering of the slower maturing plant, such as by covering the slower maturing plant with film designed to speed germination and growth or by cutting the tip of a young ear shoot to expose silk.

A further step comprises cultivating or growing the seeds of the plant. In such an embodiment, the seeds are obtained and germinated in greenhouse conditions or in the field under appropriate growth conditions to ensure that viable, healthy plants are produced. A further step comprises harvesting the seeds, near or at maturity, from the ear of the plant that received the pollen. In a particular embodiment, seed is harvested from the female parent plant, and when desired, the harvested seed can be grown to produce a progeny or first generation (F1) hybrid plant.

In a subsequent embodiment, the disclosure is related to introducing a desired trait into the progeny plant. In an aspect of the embodiment, the desired trait is selected from the group consisting of an insecticidal resistance trait, herbicide tolerant trait, disease resistance trait, yield increase trait, nutritional quality trait, agronomic increase trait, and combinations thereof. Other examples of a desired trait include modified fatty acid metabolism, for example, by transforming a plant with an antisense gene of stearoyl-ACP desaturase to increase stearic acid content of the plant. See Knultzon et al., Proc. Natl. Acad. Sci. USA 89: 2624 (1992). Decreased phytate content: (i) Introduction of a phytase-encoding gene would enhance breakdown of phytate, adding more free phosphate to the transformed plant. For example, see Van Hartingsveldt et al., Gene 127: 87 (1993), for a disclosure of the nucleotide sequence of an Aspergillus niger phytase gene. (ii) A gene could be introduced that reduces phytate content. In corn, this, for example, could be accomplished, by cloning and then reintroducing DNA associated with the single allele which is responsible for corn mutants characterized by low levels of phytic acid. See Raboy et al., Maydica 35: 383 (1990). (iii) Modified carbohydrate composition effected, for example, by transforming plants with a gene coding for an enzyme that alters the branching pattern of starch. See Shiroza et al., J. Bacteriol. 170: 810 (1988) (nucleotide sequence of Streptococcus mutans fructosyltransferase gene), Steinmetz et al., Mol. Gen. Genet. 200: 220 (1985) (nucleotide sequence of Bacillus subtillus levansucrase gene), Pen et al., Bio/Technology 10: 292 (1992) (production of transgenic plants that express Bacillus licheniformis α-amylase), Elliot et al., Plant Molec. Biol. 21: 515 (1993) (nucleotide sequences of tomato invertase genes), Sogaard et al., J. Biol. Chem. 268: 22480 (1993) (site-directed mutagenesis of barley α-amylase gene), and Fisher et al., Plant Physiol. 102: 1045 (1993) (corn endosperm starch branching enzyme II). Further examples of potentially desired characteristics include greater yield, improved stalks, enhanced root growth and development, reduced time to crop maturity, improved agronomic quality, higher nutritional value, higher starch extractability or starch fermentability, resistance and/or tolerance to insecticides, herbicides, pests, heat and drought, and disease, and uniformity in germination times, stand establishment, growth rate, maturity and kernel or seed size.

In an additional embodiment, the subject disclosure relates to a method for producing a progeny of F1 plant. Various breeding schemes may be used to produce progeny plants. In one method, generally referred to as the pedigree method, the parent may be crossed with another different plant such as a second inbred parent plant, which either itself exhibits one or more selected desirable characteristic(s) or imparts selected desirable characteristic(s) to a hybrid combination. If the two original parent plants do not provide all the desired characteristics, then other sources can be included in the breeding population. Progeny plants, that is, pure breeding, homozygous inbred lines, can also be used as starting materials for breeding or source populations from which to develop progeny plants.

Thereafter, resulting seed is harvested and resulting progeny plants are selected and selfed or sib-mated in succeeding generations, such as for about 5 to about 7 or more generations, until a generation is produced that no longer segregates for substantially all factors for which the inbred parents differ, thereby providing a large number of distinct, pure-breeding inbred lines.

In another embodiment for generating progeny plants, generally referred to as backcrossing, one or more desired traits may be introduced into the parent by crossing the parent plants with another parent plant (referred to as the donor or non-recurrent parent) which carries the gene(s) encoding the particular trait(s) of interest to produce F1 progeny plants. Both dominant and recessive alleles may be transferred by backcrossing. The donor plant may also be an inbred, but in the broadest sense can be a member of any plant variety or population cross-fertile with the recurrent parent. Next, F1 progeny plants that have the desired trait are selected. Then, the selected progeny plants are crossed with the fertile parent to produce backcross progeny plants. Thereafter, backcross progeny plants comprising the desired trait and the physiological and morphological characteristics of the fertile parent are selected. This cycle is repeated for about one to about eight cycles, preferably for about three or more times in succession to produce selected higher backcross progeny plants that comprise the desired trait and all of the physiological and morphological characteristics of the parent or restored fertile parent when grown in the same environmental conditions. Exemplary desired trait(s) include insect resistance, enhanced nutritional quality, waxy starch, herbicide resistance, yield stability, yield enhancement and resistance to bacterial, fungal and viral disease. One of ordinary skill in the art of plant breeding would appreciate that a breeder uses various methods to help determine which plants should be selected from the segregating populations and ultimately which inbred lines will be used to develop hybrids for commercialization. In addition to the knowledge of the germplasm and other skills the breeder uses, a part of the selection process is dependent on experimental design coupled with the use of statistical analysis. Experimental design and statistical analysis are used to help determine which plants, which family of plants, and finally which inbred lines and hybrid combinations are significantly better or different for one or more traits of interest. Experimental design methods are used to assess error so that differences between two inbred lines or two hybrid lines can be more accurately determined. Statistical analysis includes the calculation of mean values, determination of the statistical significance of the sources of variation, and the calculation of the appropriate variance components. Either a five or a one percent significance level is customarily used to determine whether a difference that occurs for a given trait is real or due to the environment or experimental error. One of ordinary skill in the art of plant breeding would know how to evaluate the traits of two plant varieties to determine if there is no significant difference between the two traits expressed by those varieties. For example, see Fehr, Walt, Principles of Cultivar Development, p. 261-286 (1987) which is incorporated herein by reference. Mean trait values may be used to determine whether trait differences are significant, and preferably the traits are measured on plants grown under the same environmental conditions.

This method results in the generation of progeny, F1 inbred plants with substantially all of the desired morphological and physiological characteristics of the recurrent parent and the particular transferred trait(s) of interest. Because such progeny inbred plants are heterozygous for loci controlling the transferred trait(s) of interest, the last backcross generation would subsequently be selfed to provide pure breeding progeny for the transferred trait(s).

Backcrossing may be accelerated by the use of genetic markers such as SSR, RFLP, SNP or AFLP markers to identify plants with the greatest genetic complement from the recurrent parent.

Direct selection may be applied where a single locus acts as a dominant trait, such as the herbicide resistance trait. For this selection process, the progeny of the initial cross are sprayed with the herbicide before the backcrossing. The spraying eliminates any plants which do not have the desired herbicide resistance characteristic, and only those plants which have the herbicide resistance gene are used in the subsequent backcross. In the instance where the characteristic being transferred is a recessive allele, it may be necessary to introduce a test of the progeny to determine if the desired characteristic has been successfully transferred. The process of selection, whether direct or indirect, is then repeated for all additional backcross generations.

It should be appreciated by those having ordinary skill in the art that backcrossing can be combined with pedigree breeding as where the parent plant is crossed with another plant, the resultant progeny are crossed back to the first parent and thereafter, the resulting progeny of this single backcross are subsequently inbred to develop new inbred lines. This combination of backcrossing and pedigree breeding is useful as when recovery of fewer than all of the parent characteristics than would be obtained by a conventional backcross are desired.

The subject disclosure also relates to one or more plant parts. In an embodiment, plant parts include plant cells, plant protoplasts, plant cell tissue cultures from which plants can be regenerated, plant DNA, plant calli, plant clumps, and plant cells that are intact in plants or parts of plants, such as embryos, pollen, ovules, flowers, seeds, kernels, ears, cobs, leaves, husks, stalks, roots, root tips, brace roots, lateral tassel branches, anthers, tassels, glumes, silks, tillers, and the like.

In subsequent embodiments, the subject disclosure relates to a plant regenerated form a plant cell. Further embodiments include a plant comprising the plant cell. In some embodiments the plant may be a monocotyledonous or dicotyledonous plant. In other embodiments, the monocotyledonous plant is a maize plant. Additional embodiments include a plant part, plant tissue, or plant seed.

In other embodiments, the subject disclosure is in reference to a plant cell. The term “cell” as referred to herein encompasses a living organism capable of self replication, and may be a cell of a eukaryotic organism classified under the kingdom Plantae. In some embodiments the cell is a plant cell. In some embodiments, the plant cell can be but is not limited to any higher plant, including both dicotyledonous and monocotyledonous plants, and consumable plants, including crop plants and plants used for their oils. Thus, any plant species or plant cell can be selected as described further below.

In some embodiments, plant cells in accordance with the present disclosure includes, but is not limited to, any higher plants, including both dicotyledonous and monocotyledonous plants, and particularly consumable plants, including crop plants. Such plants can include, but are not limited to, for example: alfalfa, soybeans, cotton, rapeseed (also described as canola), linseed, corn, rice, brachiaria, wheat, safflowers, sorghum, sugarbeet, sunflowers, tobacco and turf grasses. Thus, any plant species or plant cell can be selected. In embodiments, plant cells used herein, and plants grown or derived therefrom, include, but are not limited to, cells obtainable from rapeseed (Brassica napus); indian mustard (Brassica juncea); Ethiopian mustard (Brassica carinata); turnip (Brassica rapa); cabbage (Brassica oleracea); soybean (Glycine max); linseed/flax (Linum usitatissimum); maize (also described as corn) (Zea mays); safflower (Carthamus tinctorius); sunflower (Helianthus annuus); tobacco (Nicotiana tabacum); Arabidopsis thaliana; Brazil nut (Betholettia excelsa); castor bean (Ricinus communis); coconut (Cocus nucifera); coriander (Coriandrum sativum); cotton (Gossypium spp.); groundnut (Arachis hypogaea); jojoba (Simmondsia chinensis); oil palm (Elaeis guineeis); olive (Olea eurpaea); rice (Oryza sativa); squash (Cucurbita maxima); barley (Hordeum vulgare); sugarcane (Saccharum officinarum); rice (Oryza sativa); wheat (Triticum spp. including Triticum durum and Triticum aestivum); and duckweed (Lemnaceae sp.). In some embodiments, the genetic background within a plant species may vary.

Some embodiments of the subject disclosure also provide commodity products, for example, a commodity product produced from a transgenic plant or seed. Commodity products may include, for example and without limitation: food products, protein concentrate, fiber, meals, oils, flour, or crushed or whole grains or seeds of a plant or a transgenic plant of the subject disclosure. The detection of one or more nucleotide sequences encoding a polypeptide comprising a transgene in one or more commodity or commodity products is de facto evidence that the commodity or commodity product was at least in part produced from a transgenic plant of the subject disclosure. In particular embodiments, a commodity product of the invention comprise a detectable amount of a nucleic acid sequence encoding a polypeptide comprising a transgene. In some embodiments, such commodity products may be produced, for example, by obtaining transgenic plants and preparing food or feed from them.

Embodiments of the subject disclosure are further exemplified in the following Examples. It should be understood that these Examples are given by way of illustration only. From the above embodiments and the following Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims. The following is provided by way of illustration and not intended to limit the scope of the invention.

### EXAMPLES

### Example 1: Design and Construction of Tobacco Gene Expression Cassettes

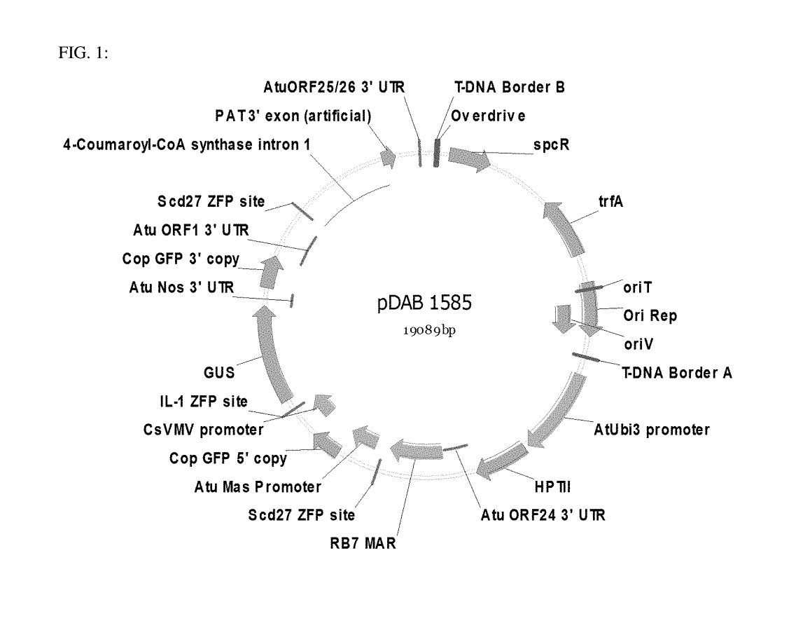

The pDAB1585 (FIG. 1) binary plasmid was constructed. This plasmid vector contained several gene expression cassettes and site specific nuclease recognition sequences for targeting of donor polynucleotide sequences. The first gene expression cassette contained the Arabidopsis thaliana Ubiquitin 3 promoter (At Ubi3 promoter) operably linked to the hygromycin resistance gene (HPTII), and was terminated by the Agrobacterium tumefaciens ORF24 3′ UTR termination sequence (Atu ORF 24 3′ UTR). This gene expression cassette was followed by a RB7 matrix attachment region (RB7 MAR), and the Scd27 site specific nuclease recognition sequence (Scd27 ZFP site). Four tandem repeats of recognition sequences (i.e. Scd27 ZFN binding sites) flanked the MAR and 4-CoAS intron sequences. The binding sites were palindromic sequences (SEQ ID NO:28; GCTCAAGAACAT and SEQ ID NO:29; TACAAGAACTCG), such that only a single ZFN needed to be expressed for the Fok1 nuclease domain to dimerize at the cleavage site. A second gene expression cassette contained the Agrobacterium tumefaciens Delta mas promoter (Atu Mas promoter) operably linked to a truncated fragment of the 5′ end of the green fluorescent protein gene (Cop GFP 5′ copy), that was operably linked to the IL-1 site specific nuclease recognition sequence (IL-1 ZFP site of SEQ ID NO:16; ATTATCCGAGTTCACCAGAACTCGGATAAT and SEQ ID NO:30; ATTATCCGAGTTCTGGTGAACTCGGATAAT), that was operably linked to the β-glucuronidase gene (GUS), and was terminated by the Agrobacterium tumefaciens nopaline synthetase 3′ UTR termination sequence (Atu Nos 3′ UTR). A third gene expression cassette contained the truncated fragment of the 3′ end of the green fluorescent protein gene (Cop GFP 3′ copy), that was operably linked to the Agrobacterium tumefaciens ORF1 3′ UTR termination sequence (Atu ORF1 3′ UTR), that was operably linked to the Scd27 site specific nuclease recognition sequence (Scd27 ZFP site), that was operably linked to the Arabidopsis thaliana 4-coumaroyl-coA-synthase intron 1, that was operably linked to the truncated fragment of the 3′ end of the phosphinothricin acetyl transferase exon (PAT 3′ exon (artificial)), and was terminated by the Agrobacterium tumefaciens ORF25/26 3′ UTR termination sequence (Atu ORF25/26 3′ UTR). This plasmid was constructed using art recognized techniques, the gene expression cassettes are disclosed as SEQ ID NO:1.

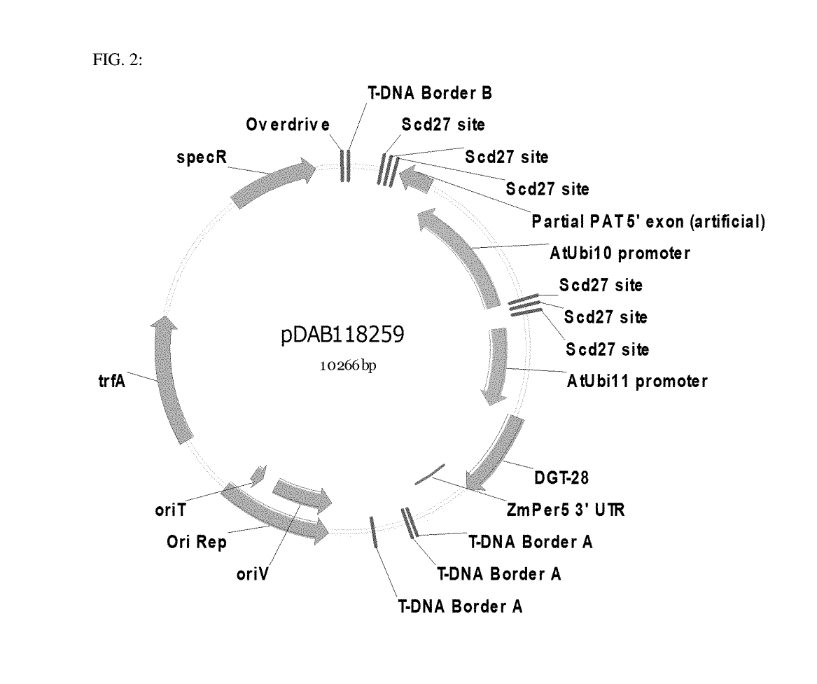

The pDAB118259 (FIG. 2) binary plasmid was constructed. This plasmid vector contained two gene expression cassettes positioned in a trans configuration with one another, and site specific nuclease recognition sequences for excision of a polynucleotide sequence to serve as a donor construct for NHEJ integration. The first gene expression cassette contained the Arabidopsis thaliana Ubiquitin 10 promoter (At Ubi10 promoter) operably linked to the 5′ end of the phosphinothricin acetyl transferase exon (PAT 5′ exon (artificial)). This gene expression cassette was flanked by repeated Scd27 site specific nuclease recognition sequence (Scd27 ZFP site). A second gene expression cassette contained the Arabidopsis thaliana Ubiquitin 11 promoter (At Ubi11 promoter) operably linked to the dgt-28 transgene (DGT-28) and was terminated to the Zea mays PER 5 3′ UTR termination sequence (ZmPer5 3′ UTR). This plasmid was constructed using art recognized techniques, the gene expression cassettes are disclosed as SEQ ID NO:2.

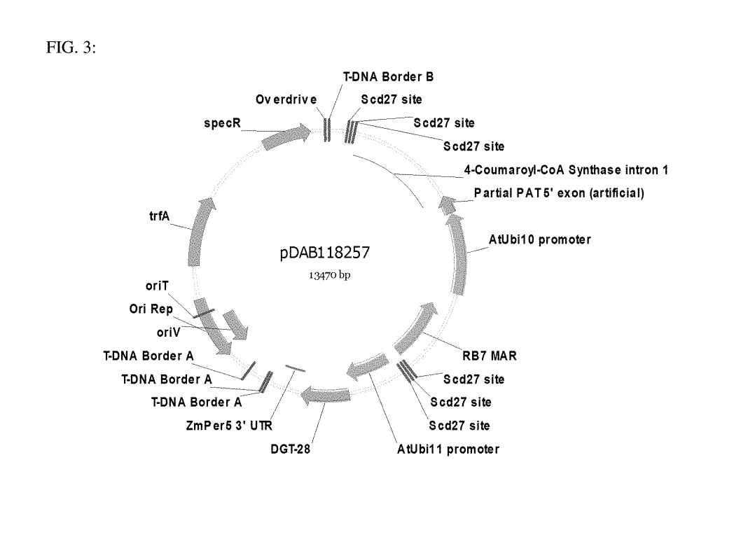

The pDAB118257 (FIG. 3) binary plasmid was constructed. This plasmid vector contained two gene expression cassettes positioned in a trans configuration with one another, and site specific nuclease recognition sequences for excision of a polynucleotide sequence to serve as a donor construct for homology directed repair integration. The first gene expression cassette contained the RB7 Matrix Attachment Region (RB7 MAR) operably linked to the Arabidopsis thaliana Ubiquitin 10 promoter (At Ubi10 promoter) operably linked to the 5′ end of the phosphinothricin acetyl transferase exon (PAT 5′ exon (artificial)) that was operably linked to the Arabidopsis thaliana 4-coumaroyl-coA-synthase intron 1. This gene expression cassette was flanked by repeated Scd27 site specific nuclease recognition sequence (Scd27 ZFP site). A second gene expression cassette contained the Arabidopsis thaliana Ubiquitin 11 promoter (At Ubi11 promoter) operably linked to the dgt-28 transgene (DGT-28) that was operably linked to the Zea mays PER 5 3′ UTR termination sequence (ZmPer5 3′ UTR). This plasmid was constructed using art recognized technique, the gene expression cassettes are disclosed as SEQ ID NO:3.

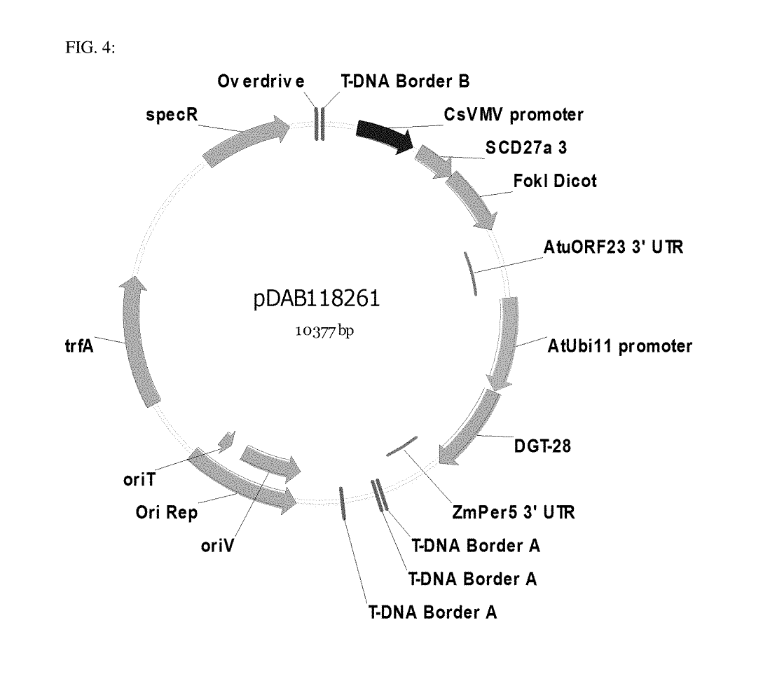

The pDAB118261 (FIG. 4) binary plasmid was constructed. This plasmid vector contained two gene expression cassettes positioned in the cis configuration with one another. The first gene expression cassette contained the cassava vein mosaic virus promoter (CsVMV promoter) operably linked to the scd27a 3 zinc finger nuclease transgene (SCD27a 3: FokI Dicot) and was terminated by the Agrobacterium tumefaciens ORF23 3′ UTR termination sequence (AtuORF23 3′ UTR). A second gene expression cassette contained Arabidopsis thaliana Ubiquitin 11 promoter (At Ubi11 promoter) operably linked to the dgt-28 transgene (DGT-28) and was terminated by the Zea mays PER 5 3′ UTR termination sequence (ZmPer5 3′ UTR). This plasmid was constructed using art recognized technique, the gene expression cassettes are disclosed as SEQ ID NO:4.

### Example 2: Design of Zinc Finger Proteins

Zinc finger proteins directed against the identified DNA recognition sequences of SCD27 and IL-1 were designed as previously described. See, e.g., Urnov et al., (2005) Nature 435:646-551. Exemplary target sequence and recognition helices and recognition sequences were originally provided in U.S. Pat. No. 9,428,756 and U.S. Pat. No. 9,187,758 (the disclosure of which are herein incorporated by reference in their entirety). Zinc Finger Nuclease (ZFN) recognition sequences were designed for the previously described recognition sequences. Numerous ZFP designs were developed and tested to identify the fingers which bound with the highest level of efficiency with the recognition sequences of the recognitions sequences. The specific ZFP recognition helices which bound with the highest level of efficiency to the zinc finger recognition sequences were used for targeting and integration of a donor sequence within the Zea mays genome.

The Scd27 and IL-1 zinc finger designs were incorporated into zinc finger expression vectors encoding a protein having at least one finger with a CCHC structure. See, U.S. Patent Publication No. 2008/0182332. In particular, the last finger in each protein had a CCHC backbone for the recognition helix. The non-canonical zinc finger-encoding sequences were fused to the nuclease domain of the type IIS restriction enzyme FokI (amino acids 384-579 of the sequence of Wah et al., (1998) Proc. Natl. Acad. Sci. USA 95:10564-10569) via a four amino acid ZC linker and an opaque-2 nuclear localization signal derived from Zea mays to form zinc-finger nucleases (ZFNs). See, U.S. Pat. No. 7,888,121. Zinc fingers for the various functional domains were selected for in vivo use. Of the numerous ZFNs that were designed, produced and tested to bind to the putative genomic target locus, the ZFNs described above were identified as having in vivo activity and were characterized as being capable of efficiently binding and cleaving the unique polynucleotide recognition sequences within the target locus in planta.

The above described plasmid vector containing the ZFN gene expression constructs were designed and completed using skills and techniques commonly known in the art (see, for example, Ausubel or Maniatis). Each ZFN-encoding sequence was fused to a sequence encoding an opaque-2 nuclear localization signal (Maddaloni et al., (1989) Nuc. Acids Res. 17:7532), that was positioned upstream of the zinc finger nuclease. The non-canonical zinc finger-encoding sequences were fused to the nuclease domain of the type IIS restriction enzyme FokI (amino acids 384-579 of the sequence of Wah et al. (1998) Proc. Natl. Acad. Sci. USA 95:10564-10569). Expression of the fusion proteins was driven by a strong constitutive promoter. The expression cassette also included the 3′ UTR (comprising the transcriptional terminator and polyadenylation site). The self-hydrolyzing 2A encoding the nucleotide sequence from Thosea asigna virus (Szymczak et al., (2004) Nat Biotechnol. 22:760-760) was added between the two Zinc Finger Nuclease fusion proteins that were cloned into the construct.

### Example 3: Tobacco Plant Transformation

The pDAB1585 construct was stably transformed into tobacco via random integration using Agrobacterium co-cultivation. Seed from tobacco plants was surface sterilized by soaking for 10 minutes in 20% Clorox® solution and rinsed twice in sterile water. Tobacco plants were grown aseptically in TOB-medium (Phytotechnology Laboratories, Shawnee Mission, Kans.) with 30 g/L sucrose solidified with 8 g/L TC Agar (Phytotechnology Laboratories) in PhytaTrays® (Sigma, St. Louis, Mo.) at 28° C. and a 16/8 hour light/dark photoperiod (60 μmol m2 sec2). To make transgenic plant events with integrated donor constructs, leaf discs (1 cm2) were cut and incubated in an overnight culture of Agrobacterium tumefaciens strain LBA4404 harboring plasmids pDAB188257 or pDAB188259, grown to OD600˜1.2 nm, blotted dry on sterile filter paper, and then placed onto TOB+MS medium (Phytotechnology Laboratories) and 30 g/L sucrose with the addition of 1 mg/L indoleacetic acid and 1 mg/L benzyaminopurine solidified with 8 g/L TC Agar (Phytotechnology Laboratories)—in 100×20 mm dishes (10 discs per dish) sealed with Nescofilm® (Karlan Research Products Corporation, Cottonwood, Ariz.). Following 72 hours of co-cultivation, leaf discs were transferred to TOB+250Ceph+50KAN, which is the same medium with 250 mg/L cephotaxime and 50 mg/L Kanamycin (Phytotechnology Laboratories). After 3 to 4 weeks, plantlets were transferred to TOB-250Ceph+50 KAN MS medium with 250 mg/L cephotaxime and 50 mg/L kanamycin—in PhytaTrays for an additional 3 to 4 weeks prior to leaf sampling and molecular analysis. Green plants displaying shoot elongation and root growth on medium with 50 mg/L Kanamycin were then be sampled for molecular analysis. Sampling involved cutting leaf tissue with a sterile scalpel and placing either 1-2 cm2 into 1.2 mL cluster tubes for PCR analysis or 3-4 cm2 into 2.0 mL Safe Lock tubes (Eppendorf, Hauppauge, N.Y.) for Southern blot analysis surrounded by dry ice for rapid freezing. The tubes were then be covered in 3M™ Micropore™ tape (Fisher Scientific, Nazareth, Pa.) and lyophilized for 48 hours in a Virtual XL-70 (VirTis, Gardiner, N.Y.). Once the tissue was lyophilized, the tubes were capped and stored at 8° C. until analysis. Three single copy, intact events were selected for each construct based on qPCR and Southern blot analysis and regenerated T0 plants were transferred to the greenhouse and allowed to self-pollinate.

Transformants were obtained and confirmed via molecular confirmation. Transgenic plants containing a single copy, homozygous T2 target line with a non-functional herbicide resistance gene flanked by ZFN cleavage sites were developed. This target line containing the T-strand of pDAB1585 was developed for use in establishing proof of concept for targeted transgene integration via homology-directed repair. Briefly, the tobacco RB7 matrix attachment region (MAR) and the Arabidopsis thaliana 4-coumaryl synthase intron-1 (4-CoAS) served as sequences homologous to incoming donor DNA. A 3′ fragment of the phosphinothricin acetyltransferase (PAT) gene was included for in vitro selection following targeted donor integration. Four tandem repeats of ZFN binding sites (Scd27) flanked the MAR and 4-CoAS intron sequences. The binding sites were palindromic sequences (SEQ ID NO:28; GCTCAAGAACAT and SEQ ID NO:29; TACAAGAACTCG) such that only a single ZFN needed to be expressed for the Fok1 nuclease domain to dimerize at the cleavage site.

Next, the donor constructs (i.e., pDAB118257, HDR Donor and pDAB118259, NHEJ Donor) were individually transformed into the transgenic pDAB1585 tobacco plants using the previously described transformation method. Transgenic plants that contained both a T-strand fragment for pDAB1585 and a second T-strand fragment for either pDAB118257 or pDAB118259 were obtained and confirmed via molecular confirmation using qPCR and Southern blot analysis. The regenerated T0 plants were transferred to the greenhouse and allowed to self-pollinate.

Finally, the zinc finger nuclease construct (i.e., pDAB118261) was transformed into tobacco plants using the previously described transformation method. Transgenic plants that contained a T-strand fragment for pDAB118261 were obtained and confirmed via molecular confirmation using qPCR and Southern blot analysis. The regenerated T0 plants were transferred to the greenhouse and allowed to self-pollinate.

Samples of the T1 progeny (˜25 seed) from self-pollination of each selected T0 Donor/Target and ZFN plant were germinated aseptically on TOB-medium and, following qPCR analysis, homozygous individuals (along with a few nulls to serve as controls) were selected, transferred to the greenhouse and used for crossing to produce F1 progeny.

### Example 4: Crossing of Tobacco Plants

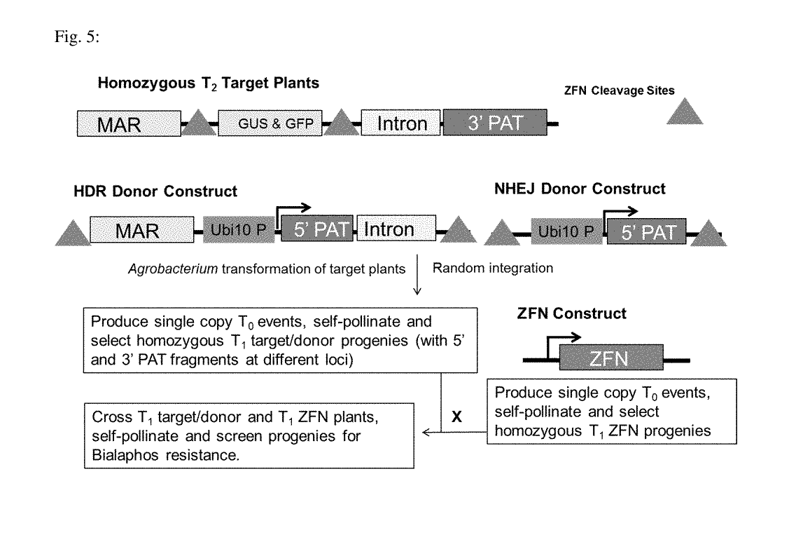

Crossing among the homozygous T1 Donor/Target and ZFN (and null) plants (FIG. 5) was made using controlled pollination. Pollen from the anthers of Donor/Target plants was introduced to the stigma of ZFN (and null) plants and vice versa to generate all possible combinations among the independent events. Plants used as females were emasculated (i.e., anthers removed prior to dehiscence) using forceps ˜15-30 minutes prior to being pollinated. Flowers were selected for emasculation by observing the anthers and the flower color. Newly opened flowers were bright pink around the edges and the anthers were still closed. Flowers containing dehised anthers were not used. Multiple flowers from a single inflorescence were emasculated and pollinated. Anthers from the male parent were removed using forceps and rubbed onto the sticky receptive stigma, until the stigma was coated with pollen. Flowers were then labeled with a pollination tag listing the cross made and the pollination date. When the capsules were brown and dry, they were harvested and the progeny seed removed.

A sample (˜25 seed) of F1 progeny from each (Donor/Target)×ZFN (and null) cross was germinated aseptically on TOB-medium and leaf discs were plated onto TOB+250Ceph+5BASTA-MS medium with 30 g/L sucrose with the addition of 1 mg/L indoleacetic acid and 1 mg/L benzyaminopurine solidified with 8 g/L TC Agar in 100×20 mm dishes (10 discs per dish) sealed with Nescofilm®. Leaf samples from regenerated plants were sampled and analyzed for targeted integration using in-out PCR and Southern blot analysis. A few plants from each cross were transferred to the greenhouse and allowed to self-pollinate to generate F2 progenies for additional screening via glufosinate selection and molecular confirmation.

### Example 5: Molecular Confirmation

Transgene copy number determination and Transcription analysis by hydrolysis probe assay was performed by real-time PCR using the LIGHTCYCLER®480 system (Roche Applied Science, Indianapolis, Ind.). Assays were designed for the gene of interest (PAT and NPTII for copy number and FokI for expression) and the internal reference gene (PalA for copy number and elf1α for expression) (GenBank ID: AB008199 and Genbank Accession No: XM_009595030) using LIGHTCYCLER® Probe Design Software 2.0. For amplification, LIGHTCYCLER®480 Probes Master mix (Roche Applied Science, Indianapolis, Ind.) was prepared at 1× final concentration in a 10 μL volume multiplex reaction containing 0.4 μM of each primer and 0.2 μM of each probe (Table 1 and Table 2). A two-step amplification reaction was performed with an extension at 60° C. for 40 seconds for the selectable markers with fluorescence acquisition (Table 3).

TABLE 1
List of oligos used for gene of interest copy number/relative expression
detection.

| | | Gene or | |
|---|---|---|---|
| | | sequence | qPCR |
| Name | Oligo Sequence | of interest | usage |
| TQPATS | SEQ ID NO: 5; 5′ | PAT | Target |
| | ACAAGAGTGGATTGATGATCTAGAGAGGT 3′ | | |
| TQPATA | SEQ ID NO: 6; 5′ | PAT | Target |
| | CTTTGATGCCTATGTGACACGTAAACAGT 3′ | | |
| TQPATFQ | SEQ ID NO: 7; 5′ CY5- | PAT | Target |
| | GGTGTTGTGGCTGGTATTGCTTACGCTGG- | | |
| | BHQ2 3′ | | |
| NPTIIF | SEQ ID NO: 8; 5′ ACGACGGGCGTTCCTTG 3′ | NPTII | Target |
| NPTIIR | SEQ ID NO: 9; 5′ | NPTII | Target |
| | GAGCAAGGTGAGATGACAGGAGAT 3′ | | |
| NPTIIP_Long | SEQ ID NO: 10; 5′ 6FAM- | NPTII | Target |
| | CACTGAAGCGGGAAGGGACTGGC-BHQ1 3′ | | |
| TQPALS | SEQ ID NO: 11; 5′ | PAL | Reference |
| | TACTATGACTTGATGTTGTGTGGTGACTGA 3′ | | |
| TQPALA | SEQ ID NO: 12; 5′ | PAL | Reference |
| | GAGCGGTCTAAATTCCGACCCTTATTTC 3′ | | |
| TQPALFQ | SEQ ID NO: 13; 5′ FAM- | PAL | Reference |
| | AAACGATGGCAGGAGTGCCCTTTTTCTATCAAT- | | |
| | BHQ1 3′ | | |
| FokI_UPL_F | SEQ ID NO: 14; 5′ | FokI | Target |
| | TGAATGGTGGAAGGTGTATCC 3′ | | |
| FokI_UPL_R | SEQ ID NO: 15; 5′ | FokI | Target |
| | AAGCTGTGCTTTGTAGTTACCCTTA 3′ | | |
| UPL130 | (cat #0469366301, Roche, Indianapolis, Ind.) | FokI | Target |
| eIF1a_F | SEQ ID NO: 17; 5′ | eIF1a | Reference |
| | CCATGGTTGTTGAGACCTTCT 3′ | | |
| eIF1a_R | SEQ ID NO: 18; 5′ GCATGTCCCTCACAGCAAAA | eIF1a | Reference |
| | 3′ | | |
| eIF1a_P | SEQ ID NO: 19; 5′ AGTACCCACCATTGGGA 3′ | eIF1a | Reference |

TABLE 2
Taqman ® PCR mixture.

| Reagent | μl each | | Final Concentration |
|---|---|---|---|
| H2O | 0.6 | μL | — |
| — | — | | — |
| ROCHE 2X Master Mix | 5 | μL | 1X |
| Target Forward Primer (10 μM) | 0.4 | μL | 0.4 μM |
| Target Reverse Primer (10 μM) | 0.4 | μL | 0.4 μM |
| Target Probe (5 μM) | 0.4 | μL | 0.2 μM |
| Reference Forward Primer (10 μM) | 0.4 | μL | 0.4 μM |
| Reference Reverse Primer (10 μM) | 0.4 | μL | 0.4 μM |
| Reference Probe (5 μM) | 0.4 | μL | 0.2 μM |

TABLE 3
Thermocycler conditions for PCR amplification.

| PCR Steps | Temp (° C.) | No. of cycles |
|---|---|---|
| Step-1 | 95 | 1 |
| Step-2 | 95 | 40 |
| | 60 | |
| Step-3 | 40 | 1 |

Analysis of real time PCR data was performed using LIGHTCYCLER® software release 1.5 using the relative quant module and is based on the ΔΔCt method. For copy number, a sample of gDNA from a single copy calibrator and known two copy check were included in each run.

Tobacco plants which contained a single copy for PAT and NPTII genes via qPCR were identified and selected. These events were advanced for Southern blots analysis. Tissue samples were collected in 15 ml Eppendorf tubes and lyophilized. Tissue maceration was performed with a Geno/Grinder® 2010 (SPEX Sample Prep, Metuchen, N.J.) and a stainless steel beads. Following tissue maceration the g DNA was isolated using the NucleoSpin Plant II Midi Kit™ (Macherey-Nagel, Bethlehem, Pa.) according to the manufacturer's suggested protocol.

Genomic DNA was quantified by Quant-IT Pico Green DNA assay Kit™ (Molecular Probes, Invitrogen, Carlsbad, Calif.). Quantified gDNA was adjusted to 10 μg for the Southern blot analysis. These events were then digested with NsiI (copy number) and MfeI (PTU) restriction enzymes (New England BioLabs, Ipwich, Mass.) overnight at 37° C. followed with a clean up using Quick-Precip™ (Edge BioSystem, Gaithersburg, Md.) according to the manufacturer's suggested protocol. Events were run on a 0.8% SeaKem LE agarose Gel™ (Lonza, Rockland, Me.) at 40 volts. Then the gel was denatured, neutralized, and then transfer to a nylon charged membrane (Millipore, Bedford, Mass.) overnight. The DNA was then bound to the membrane using the UV Strata linker 1800™ (Stratagene, La Jolla, Calif.). The Blots were then prehybridized with 25 ml of DIG Easy HYB™ (Roche Indianapolis, Ind.). The probes for hybridization were labeled using the DIG System™ (Roche) according to manufactures suggested protocol. The probes were then added to the blots and incubated overnight. The blots were then washed and detected according to manufacturer's suggested protocol for DIG/CDP-Star™ (Roche). Blots were then visualized using the BioRad Gel™ doc.

### Example 6: Confirmation of Targeting and Intragenic Recombination in Tobacco Via NHEJ and HDR

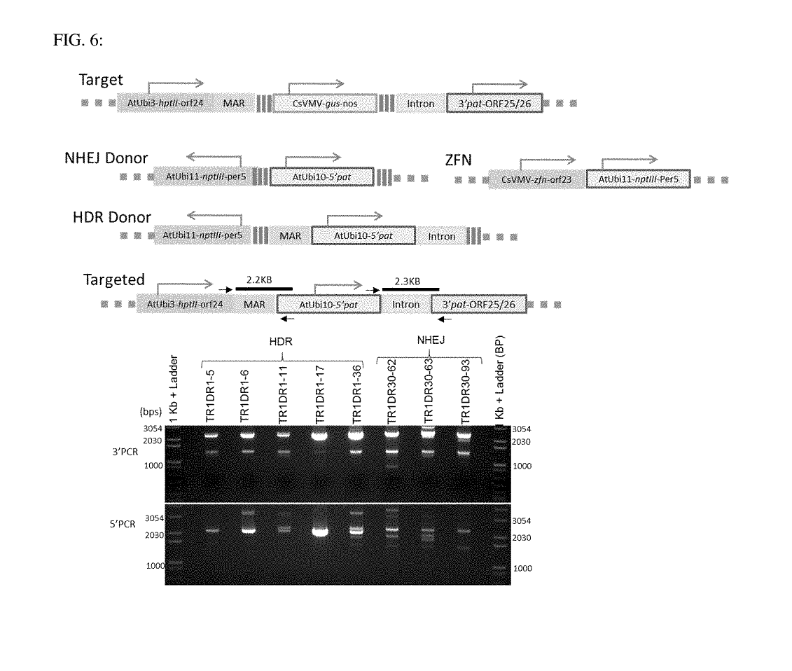

The results indicated that tobacco plants can utilize the NHEJ directed repair mechanism to mobilize a donor DNA from one parent into a site specific genomic locus within the progeny plants (F1 plants). Accordingly, transgenic plants containing the integrated 3′ partial pat selectable marker gene flanked by ZFN cleavage recognition sites (from pDAB1585) served as the target genomic locus. These transgenic plants also contained the corresponding 5′ partial pat sequence (with or without any flanking homology arms or any other regions of homology) and were flanked by ZFN cleavage sites (from pDAB118257 or pDAB118259) that served as the donor DNA sequences. Upon crossing the above described transgenic plant with a second transgenic plant containing a ZFN-expressing event (from pDAB118261), the ZFN liberated the donor by cleaving the recognition sequence (e.g., Scd27 site), and also creating a double strand break at the genomic locus (at the Scd27 site of the pDAB1585 T-strand integration) that was integrated within the first transgenic plant. Next, the donor gene (e.g., pat) integrated within the site specific locus via a NHEJ or HDR mediated recombination mechanism (FIG. 6). The concurrent cleavage and integration of the target and donor within the progeny plants occurred at all cell cycle stages (G1, S, G2, and M), thereby resulting in donor mobilization into the target locus via an NHEJ mediated process and functionalization of the pat selectable marker gene.

The insertion of the dgt-28 donor DNA within the target line was hypothesized to occur in one of two orientations. The integration of the dgt-28 transgene and the orientation of this integration were confirmed with an “In-Out” PCR assay. The In-Out PCR assay utilizes an “Out” primer that was designed to bind to the target Oryzae sativa ubiquitin 3 promoter sequence. In addition, an “In” primer was designed to bind to the dgt-28 donor sequence. The amplification reactions which were completed using these primers only amplify a donor gene which is inserted at the target locus. The resulting PCR amplicon was produced from the two primers, and consisted of a sequence that spanned the junction of the insertion. Positive and negative controls were included in the assay.

An end point PCR was utilized to detect the above described sequences. The PCR reactions were conducted using ˜25 ng of template genomic DNA, 0.2 uM dNTPs, 0.4 uM forward and reverse primers, and 0.25 ul of Ex Taq HS polymerase. Reactions were completed in three steps: the first step consisted of one cycle at 94° C. (3 minutes) and 35 cycles at 94° C. (30 seconds), 68° C. (30 seconds) and 72° C. (2 minutes). The amplicons were sequenced to confirm that the pat gene had integrated within the target line. In addition the amplicons of the 5′ In-Out PCR were diluted and run on a 1% TAE gel and visualized using BioRad Gel doc software to identify the events containing the expected amplicon sizes of about 2.6 Kb.

5′ and 3′ in-Out PCR Detection

The insertion of the pat donor DNA within the target line was hypothesized to occur in one of two orientations (FIG. 6). The integration of the pat transgene and the orientation of this integration were confirmed with an In-Out PCR assay. The In-Out PCR assay utilizes an “Out” primer that was designed to bind to the target. In addition, an “In” primer was designed to bind to the donor sequence (Table 4). The amplification reactions which were completed using these primers only amplify a donor gene which is inserted at the recognition sequences of the target locus. The resulting PCR amplicon was produced from the two primers, and consisted of a sequence that spanned the junction of the insertion.

An end point PCR was utilized to detect the above described sequences. The PCR reactions were conducted using template genomic DNA and reagents described in Table 5. Reactions were completed using PCR profile described in Table 6, 7, and 8. The amplicons of the 5′ and 3′ In-Out PCR were run on a 1% TAE gel and visualized using BioRad Gel™ doc software to identify the events containing the expected amplicon sizes of about 2.2 Kb and 2.3 Kb, respectively (FIG. 6). Some amplicons were sequenced to confirm that the donor had integrated within the target line.

In total, 6 out of 200 plants showed positive 5′ or 3′ in-out PCR product for NHEJ targeting. Likewise, 15 out of 50 plants showed positive 5′ or 3′ in-out PCR product for HDR targeting. Targeted events are capable of being selected on phosphinothricin-containing medium (i.e. Liberty herbicide; Bayer CropScience, Kansas City, Mo.) by the presence of the pat gene within the event. The presence of targeted insertion events can be further confirmed by Southern blots using previously described methods.

TABLE 4
List of oligos used for in/out PCR.

| | | Primer | | |
|---|---|---|---|---|
| Name | Oligo Sequence | Location | PCR end | size |
| MAS2015 | SEQ ID NO: 20; 5′ | Insert | 5′ end | 2070 bp |
| | TGAACTTTAGGACAGAGCCA 3′ | | | |
| MAS2016 | SEQ ID NO: 21; 5′ | Target | | |
| | TGTGTATCCCAAAGCCTCA 3′ | | | |
| MAS2019 | SEQ ID NO: 22; 5′ | Insert | 3′ end | 2131 bp |
| | GCCTGGTCCATATTTAACACT 3′ | | | |
| MAS2020 | SEQ ID NO: 23; 5′ | Target | | |
| | TTGGGCTGAATTGAAGACAT 3′ | | | |

TABLE 5
PCR mixture.

| Reagent | μl each | |
|---|---|---|
| H2O | 16.35 | μL |
| 10X Buffer | 2.5 | μL |
| dNTP | 2 | μL |
| Primer (10 μM) | 1 | μL |
| Primer (10 μM) | 1 | μL |
| DNA | 2 | μL |
| Ex Taq | 0.15 | μL |

TABLE 6
Thermocycler conditions for 5′ end PCR amplification.

| PCR Steps | Temp (° C.) | Time | | No. of cycles |
|---|---|---|---|---|
| Step-1 | 94 | 2 | minutes | 1 |
| Step-2 | 98 | 12 | seconds | 35 |
| | 60 | 30 | seconds | |
| | 68 | 2 | minutes | |
| Step-3 | 72 | 10 | minutes | 1 |

TABLE 7
Thermocycler conditions for 3′ end PCR amplification.

| PCR Steps | Temp (° C.) | Time | | No. of cycles |
|---|---|---|---|---|
| Step-1 | 94 | 3 | minutes | 1 |
| Step-2 | 94 | 30 | seconds | 35 |
| | 63 | 30 | seconds | |
| | 72 | 2 | minutes | |
| Step-3 | 72 | 10 | minutes | 1 |

### Example 7: Design and Construction of Zea mays (e.g., Corn or Maize) Gene Expression Cassettes

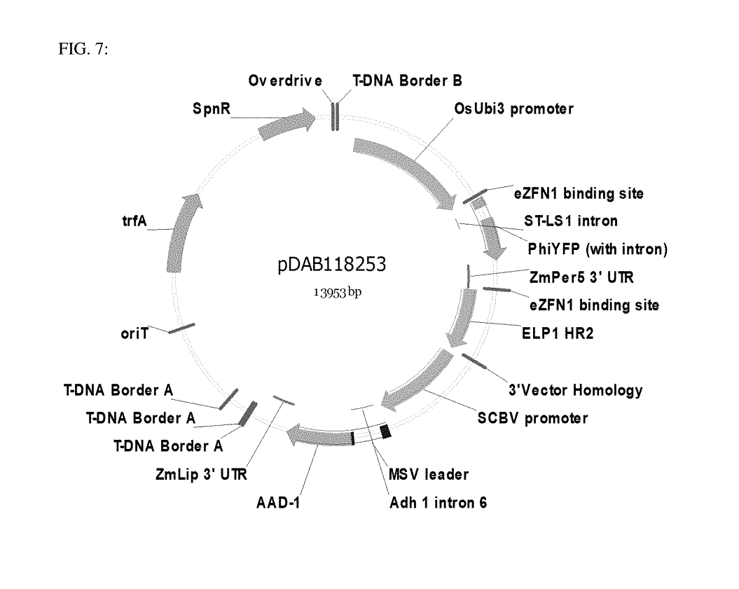

The pDAB118253 (FIG. 7) binary plasmid was constructed. This plasmid vector contained several gene expression cassettes and site specific nuclease recognition sequences for targeting of donor polynucleotide sequences. The first gene expression cassette contained the Oryza sativa Ubiquitin 3 promoter (OsUbi3 promoter) operably linked to the phi-yellow fluorescent protein gene (PhiYFP (with intron)), that contained the Solanum tuberosum LS1 intron (ST-LS1 intron), and was further operably linked to the Zea mays peroxidase 5, 3′ UTR termination sequence (ZmPer5 3′ UTR). This gene expression cassette was followed by a eZFN1 site specific nuclease recognition sequence (eZFN1 binding site of SEQ ID NO:31; CAATCCTGTCCCTAGTGGATAAACTGCAAAAGGC and SEQ ID NO:32; GCCTTTTGCAGTTTATCCACTAGGGACAGGATTG), the engineered landing pad1 sequence (ELP1 HR2), and terminated by an additional homology sequence for homology directed repair integration (3′ Vector Homology). A second gene expression cassette contained the sugar cane bacilliform virus promoter (SCBV promoter) operably linked to the aad-1 gene (AAD-1) that contained the Solanum tuberosum LS1 intron (ST-LS1 intron), and was operably linked to the Zea mays lipase 3′ UTR termination sequence (ZmLip 3′ UTR). This plasmid was constructed using art recognized technique, the gene expression cassettes are disclosed as SEQ ID NO:24.

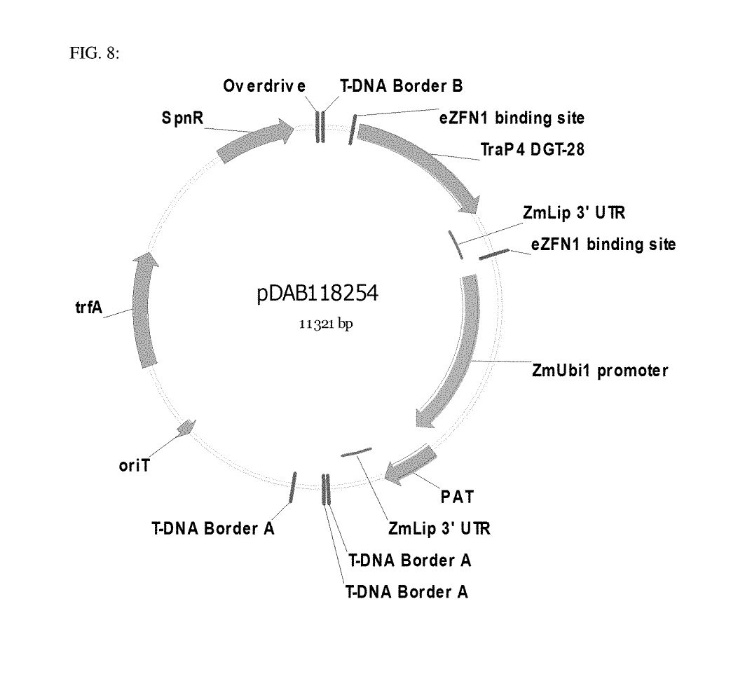

The pDAB118254 (FIG. 8) binary plasmid Non-Homologous End Joining (NHEJ) donor was constructed. This plasmid vector contained two gene expression cassettes positioned in cis with one another, and site specific nuclease recognition sequences for excision of a polynucleotide sequence to serve as a donor construct for NHEJ integration of the donor sequence into a target genomic locus. The first gene expression cassette contained the dgt-28 transgene (Trap4 DGT-28) operably linked to the Zea mays lipase 3′ UTR termination sequence (ZmLip 3′UTR). This gene expression cassette was flanked by repeated eZFN1 site specific nuclease recognition sequence (eZFN1 binding site). A second gene expression cassette contained Zea mays ubiquitin 1 promoter (ZmUbi1 promoter) operably linked to the phosphinothricin acetyltransferase transgene (PAT) that was operably linked to the Zea mays lipase 3′ UTR termination sequence (ZmLip3′ UTR). This plasmid was constructed using art recognized technique, the gene expression cassettes are disclosed as SEQ ID NO:25.

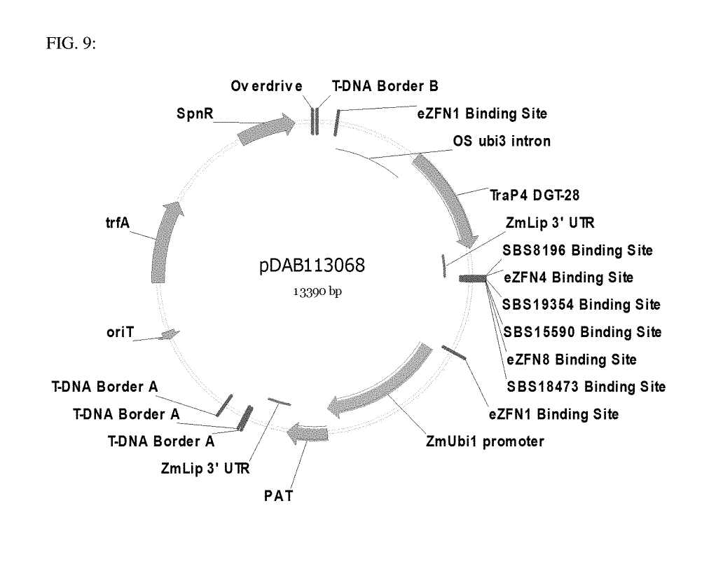

The pDAB113068 (FIG. 9) binary plasmid containing Homology-Derived Repair (HDR) donor was constructed. This plasmid vector contained two gene expression cassettes positioned in cis with one another, and site specific nuclease recognition sequences for excision of a polynucleotide sequence to serve as a donor construct for homology directed repair integration. The first gene expression cassette contained the Oryzae sativa ubiquitin 3 (Os ubi3 intron) operably linked to dgt-28 transgene (DGT-28) operably linked to the Zea mays lipase 3 3′UTR termination sequence (ZmLip 3′UTR). This gene expression cassette was flanked by repeated eZFN1 site specific nuclease recognition sequence (eZFN1 Binding Site). In addition, several additional site specific nuclease recognition sequences (e.g., SBS8196 Binding Site of SEQ ID NO:33; GCCTTTTGCAGTTT and SEQ ID NO:34; AAACTGCAAAAGGC; SBS19354 Binding Site of SEQ ID NO:35; TATGCCCGGGACAAGTG and SEQ ID NO:36; CACTTGTCCCGGGCATA; SBS15590 Binding Site of SEQ ID NO:37 CAATCCTGTCCCTA and SEQ ID NO:38; TAGGGACAGGATTG; eZFN8 Binding Site of SEQ ID NO:39 CAATCCTGTCCCTAGTGAGATGGGCGGGAGTCTT and SEQ ID NO:40 AAGACTCCCGCCCATCTCACTAGGGACAGGATTG; and, SBS18473 Binding Site of SEQ ID NO:41; TGGGCGGGAGTCTT and SEQ ID NO:42; AAGACTCCCGCCCA) were included downstream of the 3′ end of the gene expression cassette. A second gene expression cassette contained the Zea mays Ubiquitin 1 promoter (ZmUbi1 promoter) operably linked to the phosphinothricin acetyltransferase transgene (PAT) that was operably linked to the Zea mays lipase 3′ UTR termination sequence (ZmLip 3′ UTR). This plasmid was constructed using art recognized technique, the gene expression cassettes are disclosed as SEQ ID NO:26.

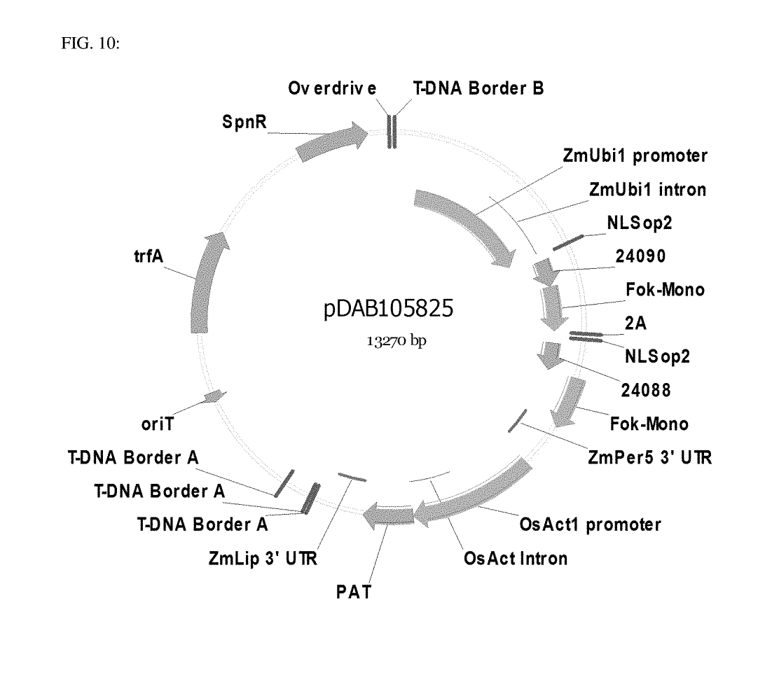

The Zinc Finger Nuclease (ZFN1) vector pDAB105825 (FIG. 10) comprised a ZFN1 coding sequence under the expression of maize Ubiquitin 1 promoter with intron1 (ZmUbi1 promoter v2) and ZmPer5 3′UTR v2 (as previously disclosed in U.S. Pat. No. 9,428,756 and U.S. Pat. No. 9,187,758, each of which are herein incorporated by reference in their entirety). A second gene expression cassette contained the Rice Actin1 (OSAct1) promoter operably linked to the phosphinothricin acetyltransferase transgene (PAT) that was operably linked to the Zea mays lipase 3′ UTR termination sequence (ZmLip 3′ UTR). This plasmid was constructed using art recognized technique.

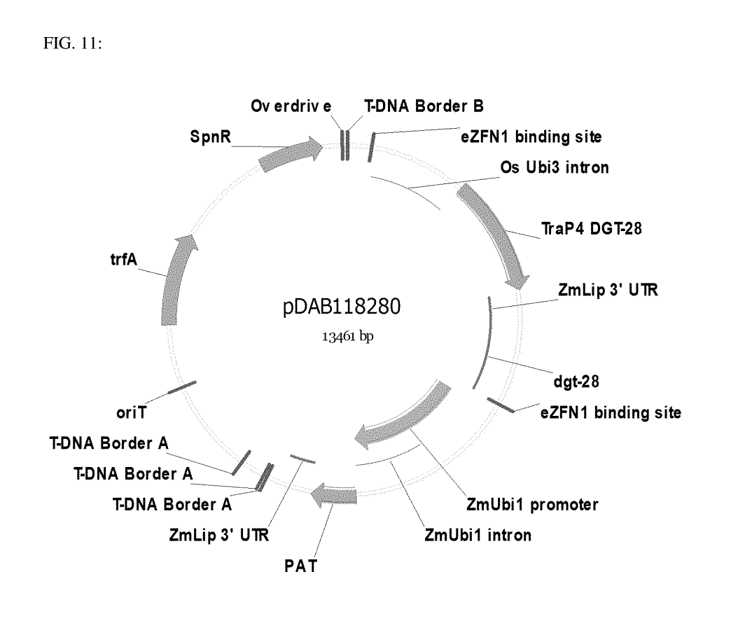

The pDAB118280 (FIG. 11) binary plasmid containing One Sided Donor (OSI) was constructed. This plasmid vector contained two gene expression cassettes positioned in cis with one another, and site specific nuclease recognition sequences for excision of a polynucleotide sequence to serve as a donor construct for homology directed repair integration. The first gene expression cassette contained the Oryza sativa ubiquitin 3 (Os ubi3 intron) operably linked to dgt-28 transgene (DGT-28) operably linked to the Zea mays lipase 3 3′UTR termination sequence (ZmLip 3′UTR). This gene expression cassette was flanked by repeated eZFN1 site specific nuclease recognition sequence (eZFN1 Binding Site). A second gene expression cassette contained the Zea mays Ubiquitin 1 promoter (ZmUbi1 promoter) operably linked to the phosphinothricin acetyltransferase transgene (PAT) that was operably linked to the Zea mays lipase 3′ UTR termination sequence (ZmLip 3′ UTR). This plasmid was constructed using art recognized technique, the gene expression cassettes are disclosed as SEQ ID NO:27

### Example 8: Design of Zinc Finger Proteins

Zinc finger proteins directed against the identified DNA recognition sequences of eZFN1 were designed as previously described. See, e.g., Urnov et al., (2005) Nature 435:646-551. Exemplary target sequence and recognition helices were previously disclosed in U.S. Pat. No. 9,428,756 and U.S. Pat. No. 9,187,758, each of which are herein incorporated by reference in their entirety. Zinc Finger Nuclease (ZFN) recognition sequences were designed for the previously described eZFN1 recognition sequences. Numerous ZFP designs were developed and tested to identify the fingers which bound with the highest level of efficiency with the recognition sequences of the plant genomic target locus. The specific ZFP recognition helices which bound with the highest level of efficiency to the zinc finger recognition sequences were used for targeting and integration of a donor sequence within the Zea mays genome.

The eZFN1 zinc finger designs were incorporated into zinc finger expression vectors encoding a protein having at least one finger with a CCHC structure. See, U.S. Patent Publication No. 2008/0182332. In particular, the last finger in each protein had a CCHC backbone for the recognition helix. The non-canonical zinc finger-encoding sequences were fused to the nuclease domain of the type IIS restriction enzyme FokI (amino acids 384-579 of the sequence of Wah et al., (1998) Proc. Natl. Acad. Sci. USA 95:10564-10569) via a four amino acid ZC linker and an opaque-2 nuclear localization signal derived from Zea mays to form zinc-finger nucleases (ZFNs). See, U.S. Pat. No. 7,888,121. Zinc fingers for the various functional domains were selected for in vivo use. Of the numerous ZFNs that were designed, produced and tested to bind to the putative genomic recognition sequence, the ZFNs used in these experiments were identified as having in vivo activity and were characterized as being capable of efficiently binding and cleaving the genomic polynucleotide recognition sequences of the genomic target locus in planta.

The above described plasmid vector containing the ZFN gene expression constructs were designed and completed using skills and techniques commonly known in the art. Each ZFN-encoding sequence was fused to a sequence encoding an opaque-2 nuclear localization signal (Maddaloni et al., (1989) Nuc. Acids Res. 17:7532), that was positioned upstream of the zinc finger nuclease. The non-canonical zinc finger-encoding sequences were fused to the nuclease domain of the type IIS restriction enzyme FokI (amino acids 384-579 of the sequence of Wah et al. (1998) Proc. Natl. Acad. Sci. USA 95:10564-10569). Expression of the fusion proteins was driven by a strong constitutive promoter. The expression cassette also included the 3′ UTR (comprising the transcriptional terminator and polyadenylation site). The self-hydrolyzing 2A encoding the nucleotide sequence from Thosea asigna virus (Szymczak et al., (2004) Nat Biotechnol. 22:760-760) was added between the two Zinc Finger Nuclease fusion proteins that were cloned into the construct.

### Example 9: Maize Transformation

The above described binary expression vectors were transformed into Agrobacterium tumefaciens strain DAt13192 ternary (U.S. Prov. Pat. No. 61/368,965). Bacterial colonies were selected and binary plasmid DNA was isolated and confirmed via restriction enzyme digestion.

Agrobacterium-Mediated Transformation of Maize

Agrobacterium-mediated transformation was used to stably integrate a chimeric gene into the plant genome and thus generate transgenic maize cells, tissues, and plants. Maize transformation methods employing binary transformation vectors are known in the art, as described, for example, in International PCT Publication No. WO2010/120452. Such methods were used to transform the maize plants for these experiments.

Transfer and Establishment of T0 Plants in the Greenhouse

Transformed plant tissues were selected on the medium containing either haloxyfop or phosphinothricin. The regenerated plants were transplanted from Phytatrays™ to small pots (T.O. Plastics, 3.5″ SVD) filled with growing media (ProMix BX; Premier Tech Horticulture), covered with humidomes (Arco Plastics Ltd.), and then hardened-off in a growth room (28° C. day/24° C. night, 16-hour photoperiod, 50-70% RH, 200 μEm-2 sec-1 light intensity). When plants reached the V3-V4 stage, they were transplanted into Sunshine Custom Blend 160 soil mixture and grown to flowering in the greenhouse (Light Exposure Type: Photo or Assimilation; High Light Limit: 1200 PAR; 16-hour day length; 27° C. day/24° C. night). Observations were taken periodically to track any abnormal phenotypes.

Production of T1 Hemizygous Seed in the Greenhouse

The resulting T0 transgenic plants were analyzed for copy number and by NGS (sequence capture method) and a subset was advanced for reciprocal crosses of the transgenic target plants (produced with the pDAB118253 binary) with the transgenic donor plants (produced with either the pDAB118254 binary or the pDAB113068 binary) to obtain T1 seed. The T1 transgenic maize plants that contained both a T-strand fragment for pDAB118253 and either pDAB118254 or pDAB113068 were obtained and confirmed via molecular confirmation using qPCR and Southern blot analysis. The obtained T1 transgenic maize plants were transferred to the greenhouse and grown to maturity. For the plasmid pDAB118280, plants homozygous to target transgene pDAB118253 were retransformed via Agrobacterium.

A subset of the T1 seed was planted and plants were analyzed for zygosity of the target/donor transgenes (containing either the pDAB118253/pDAB118254 transgenes, the pDAB118253/pDAB113068 or pDAB118253/pDAB118280 transgenes). These assays were completed using the qPCR method as described above. The qPCR reactions for PhiYFP and AAD1 were utilized to determine the zygosity of the target line, while the qPCR reactions for PAT and DGT28 were used to determine the zygosity of the donor line. From these assays 11 T1 maize plants were obtained for the cross of the pDAB118253 target line plants and pDAB118254 donor line plants. Likewise, the assays resulted in obtaining three T1 maize plants for the cross of the pDAB118253 target line plants and pDAB113068 donor line plants. These T1 plants were hemizygous for both the target and donor transgenes, and were advanced for crosses with the homozygous maize plants that contained the zinc finger nuclease for cleaving eZFN1. In total 132 plants from the pDAB118253 target line plant and pDAB118254 donor line plant crosses that were used to test for NHEJ recombination mechanism and 56 plants from the pDAB118253 target line plant and pDAB113068 donor line plant crosses that were used to test for the homology directed repair mechanism were advanced to a subsequent crossing with maize plants containing the zinc finger nuclease gene expression cassette.

### Example 10: Crossing of Maize Plants

Crossing among the Donor/Target and ZFN (and null) plants was made using controlled pollination. Eighty-eight seeds of two homozygous events that contained the ZFN gene expression cassette were planted in staggered rows to ensure that pollen shed from the pDAB118253 target line plant/pDAB118254 donor line plants or from the pDAB118253 target line plant/pDAB113068 donor line plants would fertilize the ZFN plants. Immature embryos were collected from the crossed plants.

Next the immature embryos were grown on selection medium containing glyphosate. The immature corn embryos were screened for the presence of the dgt-28 transgene to identify the immature corn embryos that contained a functional dgt-28 transgene (Table 6 and 7). In total, 83 plants were selected on regeneration medium for NHEJ targeting (Table 6), while 234 plants were regenerated for HDR targeting (Table 7). The plants were confirmed via molecular assays. The plants were tested using qPCR assays for pat, aad-1, dgt-28, and phi-yfp. The plants that did not contain the phi-yfp transgene were advanced to “In-Out” end point PCR testing. The “In-Out” PCR testing assayed immature embryos for the presence of the 5′ end of the expected recombination events. The PCR reaction was designed to amplify an amplicon spanning the Oryzae sativa ubiquitin 3 promoter and the dgt-28 coding sequence. The “In-Out” PCR testing also assayed for the 3′ end of the expected recombination events. The PCR reaction was designed to amplify an amplicon spanning the dgt-28 coding sequence and the sugar cane bacilliform virus promoter. The sugar cane bacilliform virus promoter sequence is the promoter that drives the pat selectable marker transgene. The plants that were “In-Out” PCR positive were advanced to the greenhouse and subsequently analyzed using Southern blot analyses. The presence of targeted insertion events was detected by individual In-Out PCR reactions and Southern blots using previously described methods. The expected gel fragment sizes for the PCR product and the expected Southern blot banding pattern indicated the donor sequence was excised from its original genomic location for site specific integration at another desired genomic locus.

TABLE 6
Diagnostic PCR Analysis for NHEJ Targeting in corn

| | | | | Plants | |
|---|---|---|---|---|---|
| T1 Seed | Female T0 | Male T0 | | Regen- | 5′ or 3′ |
| Batch | Parent | Parent | F1 IEs | erated | PCR + Events |
| TR1DR1 | TR1 | DR1 | 350 | 4 | 0 |
| DR2TR2 | DR2 | TR2 | 1547 | 34 | 0 |
| TR3DR3 | TR3 | DR3 | 1678 | 3 | 0 |
| DR3TR4 | DR3 | TR4 | 729 | 3 | 0 |
| DR5TR5 | DR5 | TR5 | 933 | 3 | 0 |
| DR6TR5 | DR6 | TR5 | 434 | 1 | 0 |
| DR1TR4 | DR1 | TR4 | 921 | 19 | 0 |
| TR7DR8 | TR7 | DR8 | 503 | 0 | 0 |
| DR9TR7 | DR9 | TR7 | 2891 | 4 | 0 |
| TR8DR10 | TR8 | DR10 | 263 | 12 | 11 |
| TR9DR10 | TR9 | DR10 | 290 | 0 | 0 |
| — | — | — | 10539 | 83 | 11 |
| | | | (2512*) | | |

TR—Target;
DR—Donor,
IE—Immature Embryo
*Expected 25% containing both TR and DR

TABLE 7
Diagnostic PCR Analysis for HDR Targeting in corn

| | | | | Plants | |
|---|---|---|---|---|---|
| T1 Seed | Female T0 | Male T0 | | Regen- | 5′ or 3′ |
| Batch | Parent | Parent | 1 IEs | erated | PCR + Events |
| TR10DR12 | TR10 | DR12 | 132 | 2 | 2 |
| DR13TR6 | DR13 | TR6 | 4215 | 74 | 41 |
| DR14TR11 | DR14 | TR11 | 2984 | 58 | 2 |
| — | — | — | 7331 | 234 | 75 |
| | | | (1832*) | | |

TR—Target;
DR—Donor,
IE—Immature Embryo
*Expected 25% containing both TR and DR

### Example 11: Molecular Confirmation

T0 Plants Quantitative PCR Detection and Estimation of Copy Number

Putative transgenic plantlets were analyzed for transgene copy number by quantitative real-time PCR assays using primers designed to detect relative copy numbers of the transgenes/sequences. Copy number was performed using specific TaqMan® assays for gDNA reference gene, invertase, as well as target genes aad-1, pat, ELP, dgt-28, phi-yfp, fok1 domain of the zinc finger nuclease, and specR selectable marker from the. Single copy events selected for advancement were transplanted into five gallon pots and submitted for Next Generation Sequencing (NGS) sequence capture.

Putative transgenic plantlets were analyzed for transgene copy number by quantitative real-time PCR assays using primers designed to detect relative copy numbers or relative transcription level of the transgenes/sequences. At the v1-v2 stage, small leaf tears were collected from each plant for molecular analysis. DNA was extracted using the Qiagen MagAttract Kit™ or the RNA was extracted using the Ambion MagMax® kit on Thermo KingFisherFlex™ robot (Thermo Scientific, Inc.). RNA was converted to cDNA using the Applied Biosystems High Capacity reverse transcription Kit™ with the addition of oligoTVN™. Copy number or relative transcript analysis was performed using specific TaqMan® assays for gDNA reference gene, invertase, transcript reference gene, elongation factor, as well as target genes aad-1, pat, ELP, dgt-28, phi-yfp, fok1, and specR (Table 10). The Biplex TaqMan® PCR reactions were set up according to Table 11 and running condition following Table 12. The level of fluorescence generated for each reaction was analyzed using the Roche LightCycler 480™ Real-Time PCR system according to the manufacturer's recommendations. The FAM fluorescent moiety (QPCR-TARGET) was excited at an optical density of 465/510 nm, and the HEX/VIC fluorescent moiety (QPCR-REFERENCE) was excited at an optical density of 533/580 nm. The copy number were determined by comparison of Target/Reference values for unknown samples (output by the LightCycler 480™) to Target/Reference values of known copy number standards (1-Copy: hemi; and 2-Copy: homo). Relative transcription levels were determined by the comparison of Target/Reference values, data was not further normalized.

TABLE 10
List of oligos used for gene of interest copy number/relative expression
detection of Maize.

| | | Gene or | |
|---|---|---|---|
| | | sequence | qPCR |
| Name | Oligo Sequence | of interest | usage |
| PATF | SEQ ID NO: 43; 5′ | PAT | Target |
| | ACAAGAGTGGATTGATGATCTAGAGA3′ | | |
| PATR | SEQ ID NO: 44; 5′ | PAT | Target |
| | CTTTGATGCCTATGTGACACGTAAAC | | |
| | 3′ | | |
| PATP | SEQ ID NO: 45; 5′ 6FAM- | PAT | Target |
| | CCAGCGTAAGCAATACCAGCCACAACACC- | | |
| | BHQ2 3′ | | |
| DGT28F | SEQ ID NO: 46; 5′ | DGT28 | Target |
| | TTCAGCACCCGTCAGAAT | | |
| | 3′ | | |
| DGT28R | SEQ ID NO: 47; 5′ | DGT28 | Target |
| | TGGTCGCCATAGCTTGT | | |
| | 3′ | | |
| DGT28P | SEQ ID NO: 48; 5′ 6FAM- | DGT28 | Target |
| | TGCCGAGAACTTGAGGAGGT | | |
| | BHQ 3′ | | |
| ELP1 Left_F | SEQ ID NO: 49; | ELP | Target |
| | TGGTTATGACAGGCTCCGTTTA | | |
| ELP1 Left_R | SEQ ID NO: 50; | ELP | Target |
| | AACAAACCTCCTGGCTACTTCAA | | |
| ELP1 Left_P | SEQ ID NO: 51; 5′ 6FAM | ELP | Target |
| | CTTGCTGGTGTTATGTG MGB 3′ | | |
| AAD1_F | SEQ ID NO: 52; TGTTCGGTTCCCTCTACCAA | AAD1 | Target |
| AAD1_R | SEQ ID NO: 53; CAACATCCATCACCTTGACTGA | AAD1 | Target |
| AAD1_P | SEQ ID NO: 54; 5′ 6FAM | AAD1 | Target |
| | CACAGAACCGTCGCTTCAGCAACA MGB 3′ | | |
| Mon Fok11F | SEQ ID NO: 55; 5′ | FokI | Target |
| | GTCGAGGAACTGCTCATTGG | | |
| | 3′ | | |
| Mon Fok11R | SEQ ID NO: 56; 5′ | FokI | Target |
| | CAGAAGTTGATCTCGCCGTTA | | |
| | 3′ | | |
| UPL11 | (UPL11, Roche, Indianapolis, Ind.) | FokI | Target |
| YFP_3_F | SEQ ID NO: 57; CGTGTTGGGAAAGAACTTGGA | YFP | Target |
| YFP_3_R | SEQ ID NO: 58; CCGTGGTTGGCTTGGTCT | YFP | Target |
| YFP_3_P | SEQ ID NO: 59; 5′ 6FAM CACTCCCCACTGCCT | YFP | Target |
| | MGB 3′ | | |
| Spec_F | SEQ ID NO: 60; CGCCGAAGTATCGACTCAACT | Spec | Target |
| Spec_R | SEQ ID NO: 61; GCAACGTCGGTTCGAGATG | Spec | Target |
| Spec_P | SEQ ID NO: 62; | Spec | Target |
| | TCAGAGGTAGTTGGCGTCATCGAG | | |
| EF1 NEW_F | SEQ ID NO: 63; 5′ | eF1α | Reference |
| | ATAACGTGCCTTGGAGTATTTGG | | |
| | 3′ | | |
| EF1 NEW_R | SEQ ID NO: 64; 5′ | eF1α | Reference |
| | TGGAGTGAAGCAGATGATTTGC | | |
| | 3′ | | |
| EF1 NEW_P | SEQ ID NO: 65; 5′ | eF1α | Reference |
| | MGB-Vic-TTGCATCCATCTTGTTGC | | |
| | 3′ | | |
| INV F | SEQ ID NO: 66; 5′ | Invertase | Reference |
| | TGGCGGACGACGACTTGT | | |
| | 3′ | | |
| INV R | SEQ ID NO: 67; 5′ | Invertase | Reference |
| | AAAGTTTGGAGGCTGCCGT | | |
| | 3′ | | |
| INV P | SEQ ID NO: 68; 5′ HEX- | Invertase | Reference |
| | CGAGCAGACCGCCGTGTACTT | | |
| | T-BHQ1 3′ | | |

TABLE 11
Taqman ® PCR mixture.

| Reagent | μl each | | Final Concentration |
|---|---|---|---|
| H₂O | 0.6 | μL | — |
| ROCHE or Life Technologies 2X | 5 | μL | 1X |
| Master Mix | | | |
| Target Forward Primer (10 μM) | 0.4 | μL | 0.4 μM |
| Target Reverse Primer (10 μM) | 0.4 | μL | 0.4 μM |
| Target Probe (5 μM) | 0.4 | μL | 0.2 μM |
| Reference Forward Primer (10 μM) | 0.4 | μL | 0.4 μM |
| Reference Reverse Primer (10 μM) | 0.4 | μL | 0.4 μM |
| Reference Probe (5 μM) | 0.4 | μL | 0.2 μM |

TABLE 12
Thermocycler conditions for PCR amplification.

| PCR Steps | Temp (° C.) | No. of cycles |
|---|---|---|
| Step-1 | 95 | 1 |
| Step-2 | 95 | 40 |
| | 58 | |
| | 72 | |
| Step-3 | 40 | 1 |

5′ in-Out PCR Detection (HDR-OSI)

The insertion of the dgt-28 donor DNA within the target line can occur in one of two orientations. The integration of the dgt-28 transgene and the orientation of this integration were confirmed with an “In-Out” PCR assay. The In-Out PCR assay utilizes an “Out” primer that was designed to bind to the target Oryzae sativa ubiquitin 3 promoter sequence. In addition, an “In” primer was designed to bind to the dgt-28 donor sequence. The amplification reactions which were completed using these primers only amplify a donor gene which is inserted at the genomic target locus. The resulting PCR amplicon was produced from the two primers, and consisted of a sequence that spanned the junction of the insertion. Positive and negative controls were included in the assay.

An end point PCR was utilized to detect the above described sequences. The PCR reactions were conducted using ˜25 ng of template genomic DNA, 0.2 uM dNTPs, 0.4 uM forward and reverse primers, and 0.25 ul of Ex Taq HS polymerase. Reactions were completed in three steps: the first step consisted of one cycle at 94° C. (3 minutes) and 35 cycles at 94° C. (30 seconds), 68° C. (30 seconds) and 72° C. (2 minutes). Amplicons were sequenced for a few representative plants to confirm that the dgt-28 gene had integrated within the target line. In addition the amplicons of the 5′ In-Out PCR were diluted and run on a 1% TAE gel and visualized using BioRad Gel doc software to identify the events containing the expected amplicon sizes of about 2.6 Kb.

3′ In-Out PCR Detection (HDR)

The insertion of the dgt-28 donor DNA within the target line can occur in one of two orientations. The integration of the dgt-28 transgene and the orientation of this integration were confirmed with an In-Out PCR assay. The In-Out PCR assay utilizes an “Out” primer that was designed to bind to the target sugar cane bacilliform virus promoter sequence. In addition, an “In” primer was designed to bind to the dgt-28 donor sequence. The amplification reactions which were completed using these primers only amplify a donor gene which is inserted at the genomic target locus. The resulting PCR amplicon was produced from the two primers, and consisted of a sequence that spanned the junction of the insertion. Positive and negative controls were included in the assay.

An end point PCR was utilized to detect the above described sequences. The PCR reactions were conducted using ˜25 ng of template genomic DNA, 0.2 uM dNTPs, 0.4 uM forward and reverse primers, and 0.25 ul of Ex Taq HS polymerase. Reactions were completed in three steps: the first step consisted of one cycle at 94° C. (3 minutes) and 35 cycles at 94° C. (30 seconds), 63.9° C. (30 seconds) and 72° C. (3 minutes). Amplicons were sequenced on a few representative plants to confirm that the dgt-28 gene had integrated within the target line. In addition the amplicons of the 3′ In-Out PCR were diluted and run on a 1% TAE gel and visualized using BioRad Gel doc software to identify the events containing the expected amplicon sizes of about 3.2 Kb.

3′ In-Out PCR Detection (OSI)

The insertion of the dgt-28 donor DNA within the target line can occur in one of two orientations. The integration of the dgt-28 transgene and the orientation of this integration were confirmed with an In-Out PCR assay. The In-Out PCR assay utilizes an “Out” primer that was designed to bind to the engineered land pad (ELP). In addition, an “In” primer was designed to bind to the dgt-28 donor sequence. The amplification reactions which were completed using these primers only amplify a donor gene which is inserted at the genomic target locus. The resulting PCR amplicon was produced from the two primers, and consisted of a sequence that spanned the junction of the insertion. Positive and negative controls were included in the assay.

An end point PCR was utilized to detect the above described sequences. The PCR reactions were conducted using ˜25 ng of template genomic DNA, 0.2 uM dNTPs, 0.4 uM forward and reverse primers, and 0.25 ul of Ex Taq HS polymerase. Reactions were completed in three steps: the first step consisted of one cycle at 94° C. (3 minutes) and 35 cycles at 94° C. (30 seconds), 64° C. (30 seconds) and 72° C. (2 minutes). Amplicons were sequenced on a few representative plants to confirm that the dgt-28 gene had integrated within the target line. In addition the amplicons of the 3′ In-Out PCR were diluted and run on a 1% TAE gel and visualized using BioRad Gel Doc™ software to identify the events containing the expected amplicon sizes of about 2.9 Kb.

TABLE 13
List of oligos used for in/out PCR.

| | | Primer | | |
|---|---|---|---|---|
| Name | Oligo Sequence | Location | PCR end | size |
| zmDGT28 | SEQ ID NO: 69 | Insert | 5′ end | 2614 bp |
| EP R | AGGAGGCACCACGAAAAC | | HDR/OSI | (HDR) |
| Rubi3-5 | SEQ ID NO: 70 | Target | | 2281 bp |
| | GTCAAAGAGAGGCGGCATGA | | | (OSI) |
| SCBV V3 3 | SEQ ID NO: 71 | Insert | 3′ end | 2131 bp |
| | GATTTCTGCATCACAGGTTCCTTTTG | | HDR | |
| zmDGT28 | SEQ ID NO: 72 | Target | | |
| EP F | AAGTCGATCACGGCTAGA | | | |
| zmDGT28 | SEQ ID NO: 73 | Insert | 3′ end | 2932 bps |
| EP FMOD | AAGTCGATCACGGCTAGA | | OSI | |
| ELP_Left_R | SEQ ID NO: 74 | Target | | |
| | AACAAACCTCCTGGCTACTTCAA | | | |

TABLE 14
PCR mixtures.

| | PCR mix | |
|---|---|---|
| Reagent | μl each | |
| H2O | 13.25 | μL |
| 10X Buffer | 2.5 | μL |
| dNTP | 2 | μL |
| Primer (5-10 μM) | 1 | μL |
| Primer (10 μM) | 1 | μL |
| DNA | 5 | μL |
| Ex Taq | 0.25 | μL |

TABLE 15
Thermocycler conditions for 5′ end PCR amplification.

| PCR Steps | Temp (° C.) | Time | | No. of cycles |
|---|---|---|---|---|
| Step-1 | 94 | 3 | minutes | 1 |
| Step-2 | 94 | 30 | seconds | 35 |
| | 68 | 30 | seconds | |
| | 72 | 2 | minutes | |
| Step-3 | 72 | 10 | minutes | 1 |

TABLE 16
Thermocycler conditions for 3′ HDR end PCR amplification.

| PCR Steps | Temp (° C.) | Time | | No. of cycles |
|---|---|---|---|---|
| Step-1 | 94 | 3 | minutes | 1 |
| Step-2 | 94 | 30 | seconds | 35 |
| | 63.9 | 30 | seconds | |
| | 72 | 3 | minutes | |
| Step-3 | 72 | 10 | minutes | 1 |

TABLE 17
Thermocycler conditions for 3′ OSI end PCR amplification.

| PCR Steps | Temp (° C.) | Time | | No. of cycles |
|---|---|---|---|---|
| Step-1 | 94 | 3 | minutes | 1 |
| Step-2 | 94 | 30 | seconds | 35 |
| | 64 | 30 | seconds | |
| | 72 | 2 | minutes | |
| Step-3 | 72 | 10 | minutes | 1 |

### Example 12: Confirmation of Targeting and Intragenic Recombination in Maize Via NHEJ, OSI and HDR

The results indicate that maize plants can utilize the NHEJ directed repair mechanism to mobilize a donor DNA from one parent into a site specific genomic locus. Accordingly, transgenic plants containing the integrated phi-yfp selectable marker gene flanked by ZFN cleavage recognition sites (from pDAB118253) serve as the target genomic locus. Furthermore, these transgenic plants also contained the promoterless dgt-28 transgene sequence (without any flanking homology arms or any other regions of homology) and flanked by ZFN cleavage sites (from pDAB118254) that serve as the donor DNA sequences. Upon crossing the above described transgenic plant with a second transgenic plant containing a ZFN-expressing event (from pDAB118253), the ZFN will liberate the donor by cleaving the recognition sequence (e.g., eZFN1 binding site), and also create a double strand break at the genomic locus to release the phi-yfp marker gene (at the eZFN site of the pDAB T-strand integration) that was integrated within the first transgenic plant. Next, the donor gene (e.g., dgt-28 transgene) will integrate within the site specific locus via a NHEJ mediated recombination mechanism. Successfully recombined plants can be identified for selection on glyphosate, and these plants will not express the PHI-YFP protein. The concurrent cleavage and integration of the target and donor within the progeny plants occurs at all cell cycle stages (G1, S, G2, and M), thereby resulting in donor mobilization into the genomic target locus via an NHEJ mediated process and functionalization of the pat selectable marker gene.

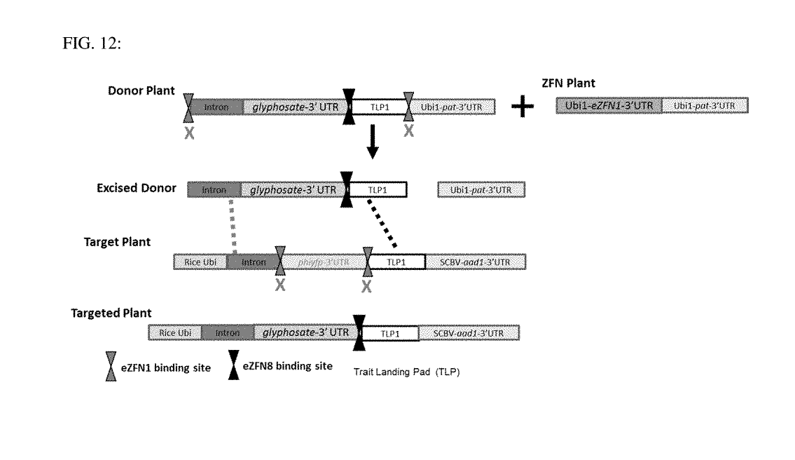

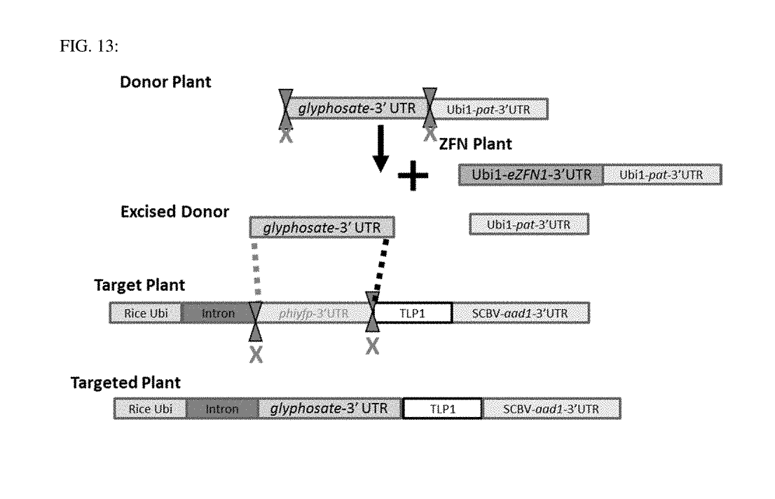

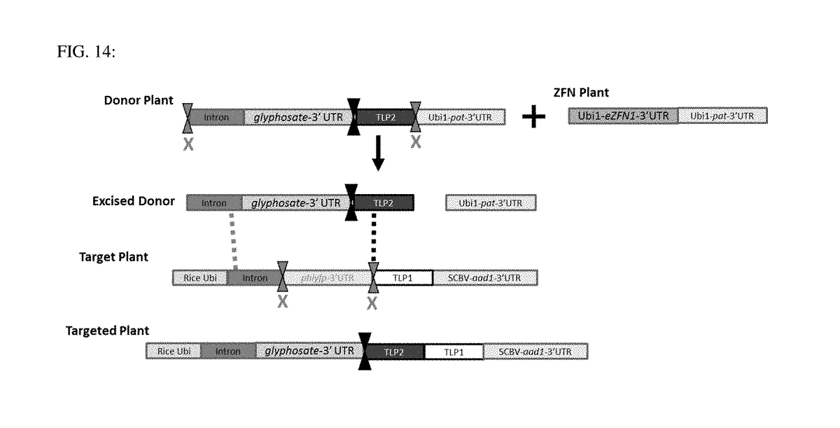

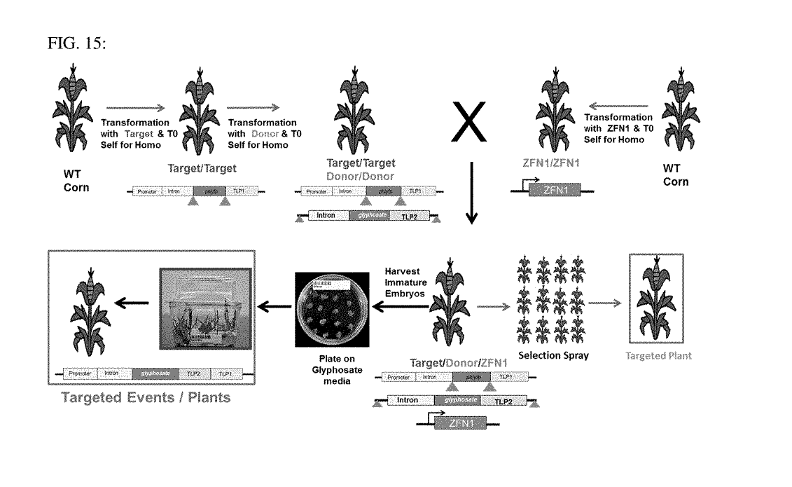

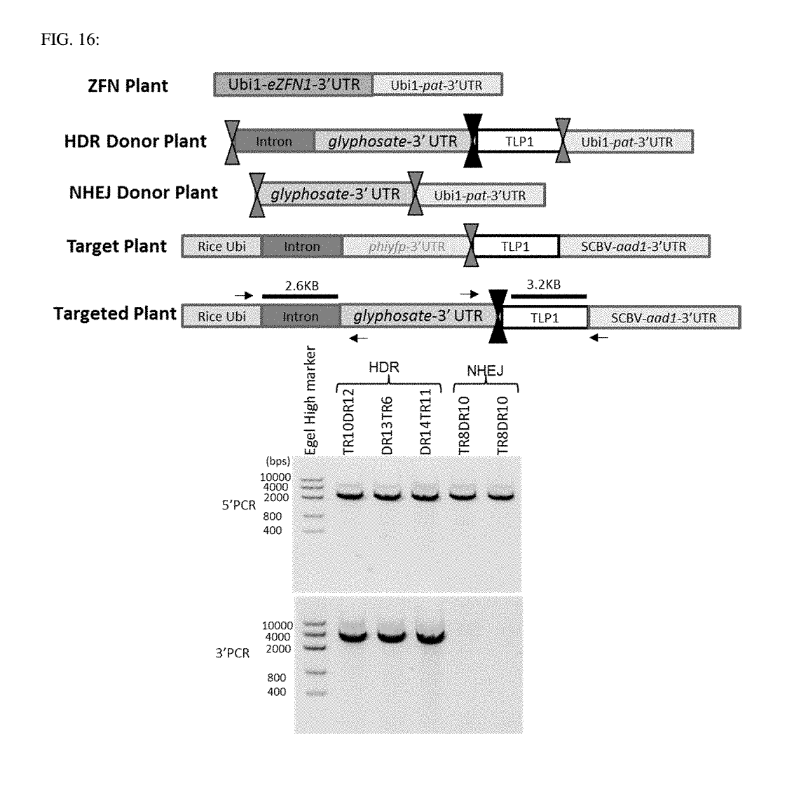

Targeted events can be selected on glyphosate-containing medium (i.e. Roundup herbicide; Monsanto, St. Louis, Mo.). The presence of targeted insertion events can be detected by individual In-out PCR reactions and Southern blots using previously described methods. The expected gel fragment sizes for the PCR product and the expected Southern blot banding patterns that indicate the presence of a targeted insertion are confirmed and progeny plants containing a properly targeted insertion of the donor within the genomic locus and selected. FIG. 12, FIG. 13, FIG. 14, and FIG. 15 provide a schematic of the intragenomic recombination process and compares the NHEJ meditated and OSI methods with the homologous recombination method. The In-Out PCR confirming HDR and NHEJ targeting is described in FIG. 16. In total, 11 In-Out PCR positive plants were obtained from NHEJ (Table 6), while 175 In-Out PCR positive plants were obtained from HDR targeting (Table 7).

### Example 13: Confirmation of Targeting and Intragenic Recombination in Maize

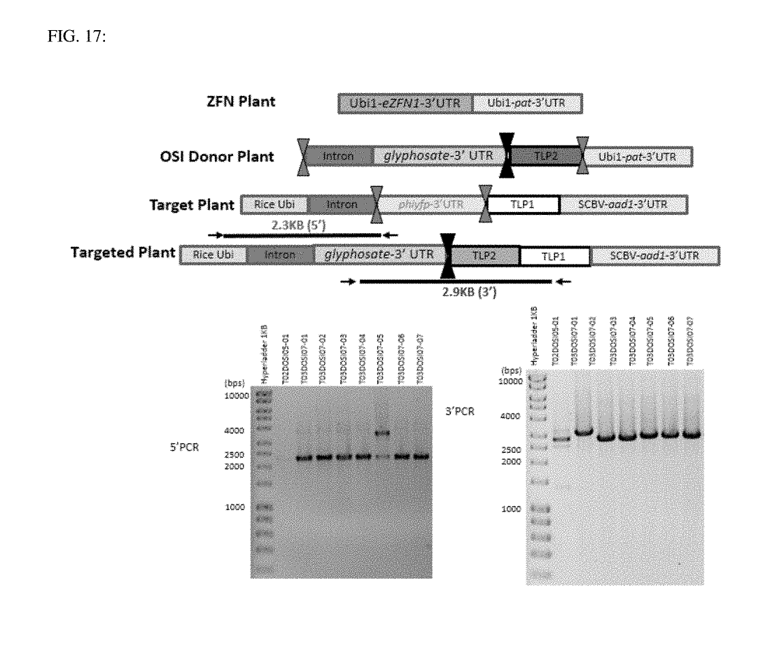

The results indicate that maize plants can utilize the NHEJ or OSI directed repair mechanism to mobilize a donor DNA from one parent into a site specific genomic locus. Accordingly, transgenic plants containing the integrated phi-yfp reporter gene operably linked to Oryza sativa Ubiquitin 3 promoter (OsUbi3 promoter) flanked by ZFN cleavage recognition sites (from pDAB118253) serve as the target genomic locus. Furthermore, these transgenic plants also contained the promoterless dgt-28 transgene sequence operably linked to intron from Oryzae sativa ubiquitin 3 (Os ubi3 intron), which provides 5′ homology to the said target genomic locus (without any flanking homology arms or any other regions of homology at 3′ end) and flanked by ZFN cleavage sites (from pDAB118280) that serve as the donor DNA sequences (FIG. 17). Upon crossing the above described transgenic plant with a second transgenic plant containing a ZFN-expressing event (from pDAB105825), the ZFN will liberate the donor by cleaving the recognition sequence (e.g., eZFN1 binding site), and also create a double strand break at the genomic locus to release the phi-yfp marker gene (at the eZFN site of the pDAB T-strand integration) that was integrated within the first transgenic plant. Next, the donor gene (e.g., dgt-28 transgene) will integrate within the site specific locus via OSI or NHEJ mediated recombination mechanism. Successfully recombined plants can be identified for selection on glyphosate, and these plants will not express the PHI-YFP protein. The concurrent cleavage and integration of the target and donor within the progeny plants occurs at all cell cycle stages (G1, S, G2, and M), thereby resulting in donor mobilization into the genomic target locus via an NHEJ mediated process and functionalization of the pat selectable marker gene.

Crossing among the Donor/Target and ZFN (and null) plants was made using controlled pollination. Homozygous events that contained the ZFN gene expression cassette were planted in staggered rows to ensure that pollen shed from the pDAB118253 target/pDAB118280 donor plants would fertilize the ZFN plants. Immature embryos were collected from the crossed plants.

Next, the immature embryos were grown on selection medium containing glyphosate. The immature corn embryos were screened for the presence of the dgt-28 transgene to identify the embryos that contained a functional dgt-28 transgene. The plants were tested using qPCR assays for pat, aad-1, dgt-28, and phi-yfp. The qPCR positive plants were advanced to “In-Out” end point PCR testing. The “In-Out” PCR testing assayed immature embryos for the presence of the 5′ end of the expected recombination events. The PCR reaction was designed to amplify an amplicon spanning the Oryzae sativa ubiquitin 3 promoter and the dgt-28 coding sequence. The “In-Out” PCR testing also assayed for the 3′ end of the expected recombination events. The PCR reaction was designed to amplify an amplicon spanning the dgt-28 coding sequence and the TLP1 sequence that is specific to Target locus (FIG. 17). The plants that were “In-Out” PCR positive were advanced to the greenhouse and subsequently analyzed using sequence analyses. In total, 66 plants selected on regeneration medium were PCR confirmed for OSI targeting, while 61 plants were confirmed for NHEJ targeting (Table 18). Selected “In-Out” PCR positive were sequence analyzed for further confirmation. The expected perfect repair at 5′ end while indels (insertion or deletion) at 3′ end further confirms the OSI-mediated site specific integration of the donor at target locus (Table 19).

TABLE 18
Diagnostic PCR analysis for OSI and NHEJ targeting in corn.

| | | | | OSI | NHEJ |
|---|---|---|---|---|---|
| | Target | Donor | IEs | (plants/ | (plants/ |
| Seed Batch | Parent | Parent | Homo | events) | events) |
| T01DOSI01 | T01 | DOSI01 | 132 | 2 (1) | 11 (4) |
| T01DOSI02 | T01 | DOSI02 | 4164 | 0 | 4 (1) |
| T01DOSI03 | T01 | DOSI03 | 2970 | 0 | 0 |
| T02DOSI04 | T02 | DOSI04 | 841 | 14 (2) | 2 (1) |
| T02DOSI05 | T02 | DOSI05 | 2374 | 8 (1) | 21 (6) |
| T03DOSI06 | T03 | DOSI06 | 447 | 3 (1) | 9 (3) |
| T03DOSI07 | T03 | DOSI07 | 940 | 39 (11) | 14 (10) |
| | | | 11868 | 66 (16) | 61 (24) |

TABLE 19
Summary of sequencing confirmation
of OSI and NHEJ targeting in corn.

| Sequencing Observations | | | | | |
|---|---|---|---|---|---|
| 5′ In/Out | 3′ In/Out | | | 5′ | 3′ |
| PCR | PCR | Plant ID | Type | In/Out | In/Out |
| Confirmed | Confirmed¹ | T01DOSI02 | OSI | + | smaller |
| | | (6B-FDB-AC1) | | | |
| Confirmed | Confirmed² | T03DOSI07 | OSI | + | + |
| | | (6B-FDB-948) | | | |
| Confirmed | Confirmed² | T03DOSI07 | OSI | + | + |
| | | (6B-FDD-552) | | | |
| Confirmed | Confirmed² | T03DOSI07 | OSI | + | + |
| | | (6B-FDD-55D) | | | |
| Confirmed | Confirmed³ | T03DOSI07 | OSI | + | + |
| | | (6B-FDB-95E) | | | |

¹1121 bp deletion at 3′ junction
²73 bp deletion 3′ junction
³117 bp insert and 73 bp deletion 3′ junction

While aspects of this invention have been described in certain embodiments, they can be further modified within the spirit and scope of this disclosure. This application is therefore intended to cover any variations, uses, or adaptations of embodiments of the invention using its general principles. Further, this application is intended to cover such departures from the present disclosure as come within known or customary practice in the art to which these embodiments pertains and which fall within the limits of the appended claims.

What is claimed is:

1. A method for inserting an integrated donor DNA within a plant genomic target locus, the method comprising:
a) providing a first viable plant containing a genomic DNA, the genomic DNA comprising the donor DNA flanked by a plurality of recognition sequences and the plant genomic target locus, wherein the plant genomic target locus comprises at least one recognition sequence;
b) providing a second viable plant containing a genomic DNA, the genomic DNA comprising a DNA encoding at least one zinc finger nuclease engineered to cleave the genomic DNA at the recognition sequence;
c) crossing the first and second viable plants such that F1 seed is produced on either the first or the second viable plant;
d) expressing the zinc finger nuclease within the F1 seed or a F1 plant, wherein the expressed zinc finger nuclease cleaves the donor DNA and the genomic DNA at the recognition sequence; and
e) growing the resultant F1 plant containing a genomic DNA, wherein the donor DNA is integrated within the recognition sequence of the plant genomic target locus via non-homologous end joining.

2. The method of claim 1, wherein the recognition sequence comprises a first and second recognition sequence.

3. The method of claim 2, wherein the first and second recognition sequences are identical.

4. The method of claim 3, wherein the zinc finger nuclease is provided by crossing the first and second viable plants such that the zinc finger nuclease cleaves both recognition sequences.

5. The method of claim 1, wherein the donor DNA and the plant genomic target locus are unlinked.

6. The method of claim 5, wherein the donor DNA and the plant genomic target locus are located on homologous chromosomes, or on non-homologous chromosomes.

7. The method of claim 1, wherein the plant genomic target locus of step a) further comprises an expression cassette located:
a) between the first and second recognition sequences; or
b) outside of the first recognition sequence; or
c) outside of the second recognition sequence.

8. The method of claim 1, wherein the first viable plant is homozygous for at least one genomic target locus or is homozygous for at least one donor DNA.

9. The method of claim 1, wherein the first viable plant is heterozygous for at least one genomic target locus or is heterozygous for at least one donor DNA.

10. The method of claim 1, wherein the plant genomic target locus is:
a) a transgenic locus; or
b) an endogenous locus.

11. The method of claim 1, wherein the zinc finger nuclease is driven by a promoter selected from the group consisting of a pollen-specific promoter, a seed-specific promoter, and a developmental-stage specific promoter.

12. The method of claim 1, wherein the donor DNA comprises a selectable marker.

13. A method for transmitting a transgene into other plants, the method comprising:
a) crossing a first plant regenerated from a plant cell or tissue transformed with an isolated nucleic acid molecule comprising a genomic target locus and the transgene with a second plant regenerated from a plant cell or tissue transformed with an isolated nucleic acid molecule comprising a promoter operably linked to a zinc finger nuclease;
b) expressing the zinc finger nuclease so that a first zinc finger nuclease monomer is paired with a second zinc finger nuclease monomer;
c) obtaining a F1 plant resulting from the cross wherein the transgene is specifically and stably integrated within the genomic target locus via non-homologous end joining; and
d) cultivating the F1 plant resulting from the cross.

14. The method of claim 13, wherein the plant regenerated from the plant cell or tissue transformed with the isolated nucleic acid molecule comprising the promoter operably linked to the zinc finger nuclease comprises at least one zinc finger nuclease monomer.

15. The method of claim 14, wherein the plant regenerated from the plant cell or tissue transformed with the isolated nucleic acid molecule comprising the promoter operably linked to the zinc finger nuclease comprises the first and the second zinc finger nuclease monomer.

16. The method of claim 13, wherein the plant regenerated from the plant cell or tissue transformed with the isolated nucleic acid molecule comprising the promoter operably linked to the zinc finger nuclease comprises the first zinc finger nuclease monomer.

17. The method of claim 16, wherein the plant regenerated from the plant cell or tissue transformed with the isolated nucleic acid molecule comprising the genomic target locus and the transgene further comprises an isolated nucleic acid molecule comprising a promoter operably linked to a second zinc finger nuclease, wherein the second zinc finger nuclease comprises the second zinc finger nuclease monomer.

18. The method of claim 13, wherein the pairing of the first and second zinc finger nuclease monomers of step b) results in the release of the transgene and cleavage of the genomic target locus.

19. The F1 plant according to claim 1 or 13, further comprising a transgenic event.

20. The F1 plant of claim 19, wherein the transgenic event comprises an agronomic trait.

21. The F1 plant of claim 20, wherein the agronomic trait is selected from the group consisting of an insecticidal resistance trait, herbicide tolerance trait, nitrogen use efficiency trait, water use efficiency trait, nutritional quality trait, DNA binding trait, small RNA trait, selectable marker trait, or any combination thereof.

22. The F1 plant of claim 20, wherein the agronomic trait comprises a herbicide tolerant trait.

23. The F1 plant of claim 22, wherein the herbicide tolerant trait comprises a dgt-28 coding sequence.

24. The F1 plant of claim 21, wherein the transgenic plant produces a commodity product.

25. The F1 plant of claim 24, wherein the commodity product is selected from the group consisting of protein concentrate, protein isolate, grain, meal, flour, oil, or fiber.

26. The F1 plant of claim 25, wherein the transgenic plant is selected from the group consisting of a dicotyledonous plant or a monocotyledonous plant.

27. The F1 plant of claim 26, wherein the monocotyledonous plant is a Zea mays plant.

28. The F1 plant of claim 26, wherein the dicotyledonous plant is a tobacco plant.
